(19) United States
(12) Patent Application Publication
(10) Pub. No.: US 2013/0298642 A1
(43) Pub. Date: Nov. 14, 2013

(54) REMOTE AIR MONITORING ARRAY SYSTEM

(71) Applicant: Logimesh IP, LLC, Cheyenne, WY (US)

(72) Inventor: William J. Gillette II, Fort Collins, CO (US)

(21) Appl. No.: 13/890,157

(22) Filed: May 8, 2013

Related U.S. Application Data
(60) Provisional application No. 61/644,093, filed on May 8, 2012.
(60) Provisional application No. 61/785,430, filed on Mar. 14, 2013.
(60) Provisional application No. 61/785,802, filed on Mar. 14, 2013.
(60) Provisional application No. 61/785,877, filed on Mar. 14, 2013.
(60) Provisional application No. 61/785,910, filed on Mar. 14, 2013.
(60) Provisional application No. 61/785,931, filed on Mar. 14, 2013.
(60) Provisional application No. 61/785,959, filed on Mar. 14, 2013.
(60) Provisional application No. 61/785,005, filed on Mar. 14, 2013.
(60) Provisional application No. 61/786,043, filed on Mar. 14, 2013.
(60) Provisional application No. 61/786,057, filed on Mar. 14, 2013.

(51) Int. Cl. G01N 33/00

(52) U.S. Cl. CPC G01N 33/0009
USPC 73/31.01

(57) ABSTRACT

An air monitoring array system can comprise a plurality of air quality sensor devices arranged within a selected area, which can be configured to measure air pollutant levels in the selected area. Furthermore, each of the plurality of air quality sensor devices can comprise at least one sensor operatively coupled to a controller, and a wireless communication device also coupled to the controller. In various embodiments, the controller can be configured to receive a measured input from the at least one sensor. Also, the wireless communication device can be configured to communicate with a central server.

### CROSS REFERENCE TO RELATED APPLICATIONS

This application claims priority to the following applications: U.S. Provisional Patent Application No. 61/644,093 filed May 8, 2012 and entitled “SYSTEMS AND METHODS FOR REMOTE ASSET MONITORING”; U.S. Provisional Patent Application No. 61/785,430 filed Mar. 14, 2013 and entitled “SYSTEMS AND METHODS FOR REMOTE ASSET MONITORING”; U.S. Provisional Patent Application No. 61/785,802 filed Mar. 14, 2013 and entitled “VALVE COVER FOR POWERING ENGINE MONITORING SYSTEM”; U.S. Provisional Patent Application No. 61/785,877 filed Mar. 14, 2013 and entitled “SYSTEM AND METHOD FOR LOGISTICALLY SETTING TANKER TRUCK ROUTES”; U.S. Provisional Patent Application No. 61/785,910 filed Mar. 14, 2013 and entitled “REMOTE VOLATILE ORGANIC COMPOUND MONITORING SYSTEM”; U.S. Provisional Patent Application No. 61/785,931 filed Mar. 14, 2013 and entitled “METHOD OF EFFICIENT BY-PRODUCT DISPOSAL BASED ON BY-PRODUCT QUALITY”; U.S. Provisional Patent Application No. 61/785,959 filed Mar. 14, 2013 and entitled “REMOTE AIR MONITORING ARRAY SYSTEM”; U.S. Provisional Patent Application No. 61/786,005 filed Mar. 14, 2013 and entitled “REMOTE MONITORING UNIT WITH VARIOUS SENSORS”; U.S. Provisional Patent Application No. 61/786,043 filed Mar. 14, 2013 and entitled “SYSTEM AND METHOD FOR REMOTELY MONITORING TOTAL DISSOLVED SOLID LEVELS”; U.S. Provisional Patent Application No. 61/786,057 filed Mar. 14, 2013 and entitled “SYSTEM AND METHOD FOR PREDICTING A NATURAL RESOURCE WELL LIFESPAN,” the respective disclosures of each of which is incorporated herein by reference.

### FIELD OF THE INVENTION

Embodiments of the present invention are directed to systems and methods for remote air monitoring and, in particular, to monitoring air quality using a monitoring array system.

### BACKGROUND OF THE INVENTION

As used herein, an “asset” may refer to any system, device, and/or machine, such as an engine or compressor, a conduit through which gas or liquid flows, an exhaust pipe, manifold, exhaust stack, liquid collection tank, or any device or machine suitable for one or more devices according to the invention to measure operating parameters.

For example, in the oil and gas market, gathering and delivering natural gas from field wells to another location for further processing requires natural gas compression via a compression package comprising of a reciprocating natural gas fired engine with a direct drive coupling to a reciprocating compressor. One typical system used in these applications is a 1,600 horsepower rotating internal combustion engine (e.g., a Caterpillar G3516, V-16 cylinder format) that is fueled by the actual field well gas (methane) that it is compressing. The engine is direct-coupled to a multi-cylinder reciprocating compressor (e.g., a Dresser-Rand 6 cylinder) therefore, if the engine RPM is 1,200 per minute, then the compressor RPM is also 1,200 per minute.

Natural gas wells require that the relatively low pressure gas extracted be compressed and piped to a facility for further processing and distribution to the respective markets. It is not unusual for a gas compression package to compress 5-10 psi natural gas from a well up to 6,000 psi for further distribution through transmission and distribution pipelines.

Ownership of the natural gas compression equipment is typically either by: (1) an owner/operator, wherein the equipment is owned directly by the gas producer (who is the well owner), or (2) a leasing company, which is an equipment leasing or rental company is contracted by the gas producer to perform the gas compression function. In the latter case, the lease is most typically price-based on the horsepower rating of the leased equipment. For example, a 4,000 HP gas compression package may be priced at $30,000 per month of service at the gas pad. A 1,600 HP unit might cost $18,000 per month for service. The gas compression packages are typically skid-mounted as they must be mobile so they can be moved in and out of service.

Immediately after a hydraulic fracturing (fracking) event, natural gas generally flows from the well at the highest flow rate. Over time, the gas flow transitions to a lower rate that may be steady for several years. Inevitably, the well will need to be stimulated, such as by fracking, to increase productivity again. Each well may be re-fracked several times over the well's life.

As of this writing, a new trend in the market is for gas producers to no longer pay for compression services on a time-based contract. Instead, the producers are switching to a “flow contract,” which is a performance-based method of paying for the gas compression package service. In essence, the producer is passing (sharing) risk to the equipment leasing/rental company. In return, they allow the company to share in the proceeds of the gas value on a performance basis. Under this “flow contract” business model, the leasing/rental company is paid for the amount of gas that is actually gathered, compressed and delivered to the transmission and distribution pipeline. Thus, the company receives payment from the producer for the amount of gas that passes through the compressor, but this amount is measured by the producer's flow meter, which is also called an EFM. Unless the leasing/rental company has a means to audit the owner's EFM data, it must accept the value provided by the producer. Thus, the leasing/rental company usually spends approximately $4,000-$6,000 for an EFM (hardware and installation) that is positioned upstream and in series with the owner's EFM. Hence, the data of the owner's EFM can be audited.

Consequently, the leasing/rental company must pay for the re-installation of its EFM every time the producer's equipment is relocated to a different well. Due to the inherent mobile nature of the producer's equipment, the frequency of re-installation could be up to once per year. Hence, the company must bear the expense of $2,000-$3,000 each time the producer moves its equipment, which can amount to about $50K-$75K over the life of the equipment. A system, device or method according to the invention can replace the EFM audit meters.

Machinery, such as internal combustion engines and compressors, have one or more inherent vibrational signatures and temperature signatures. When measured over a period, a specific vibration profile or temperature profile, or a combination of one or more of the vibration profiles and temperature profiles, can indicate the operational state of the machine. Among the vibrations signatures that may be measured are ignition detonation, valve action, crankshaft vibrations, and bearing noise.

Furthermore, by outfitting one or more individual cylinders of an internal combustion engine or gas compressor with a device that can detect and store vibrational and/or temperature measurements, one can deduce the revolutions per minute (RPM) of reciprocating machinery. As an example, if a 16-cylinder internal combustion engine (e.g., a Caterpillar model G3516B) exhibits a very specific vibration frequency and amplitude that frequency and amplitude can be associated with the spark detonation during engine operation, and one can calculate the RPM of the crankshaft by computing the time lapse between firings of the cylinders. Hence, by monitoring the vibration signature of one (1) or more cylinders, performing frequency domain processing and reviewing the resulting fast Fourier transform (FFT) signature of the vibration wave form, the RPM of the engine can be calculated.

In order to create a meaningful FFT vibrational signature, several seconds or more of sampling data can be collected in any suitable manner, such as by using an accelerometer, and then applying an algorithm using a processor, which could be a microprocessor that includes the accelerometer. As an example, an engine running at 1,200 RPMs makes 20 crankshaft revolutions per second. For a 16-cylinder engine, this equates to each cylinder detonating about every 0.8 seconds. By sampling the engine vibrations for 1 second, the resulting database would contain 20-23 revolutions worth of data, which is equal to 368 cylinder detonations. Further, there may be set maximum or minimum parameters for various vibration signals that if measured may lead to a response, such as a signal to stop or slow down the machine.

### SUMMARY OF EMBODIMENTS OF THE INVENTION

Both the foregoing summary and the following detailed description are exemplary and explanatory only and are not restrictive of the invention.

By outfitting one or more, and preferably all, of the valve covers of an engine with an autonomously-powered device wherein each device contains an accelerometer, the overall total vibration of the engine can be established. Further, the unique and independent vibration signature for each moving component associated with each engine cylinder and associated valve train (e.g., the valve lifters, rocker arms, springs, bearings) can be established. Using this technique, time-stamped vibration data can be compiled and used to determine the location of the vibration relative to the overall engine geometry and the amount of vibration.

Collecting vibration data from each cylinder simultaneously or at different times provides event based, time-related data that may be used for further analysis.

As an example, if a bearing on the top end valve train of cylinder number 12 (assuming the engine has at least twelve cylinders) was beginning to wear due to fatigue, loss of adequate lubrication, or for any other reason, the reaction forces of the valve assembly (which includes the rocker arms, springs, and lifters) would likely create additional vibration due to out-of-tolerance clearances resulting in excessive movement. This “new” vibrational FFT signature, when compared to a baseline FFT that was established during a prior calibration of the cylinder, would potentially be cause for further investigation. With scheduled data samplings (the timing of which can be of any suitable interval) performed by the end node sensor (such as every 5-60 seconds) for one or more components associated with each cylinder, a histogram can be generated that illustrates vibrational sample data over time. The same may be done for any other data, such as temperature, the composition of fuel and the composition of exhaust gas, and any or all of the various measured values may be combined in any manner to track and predict machine health.

By setting upper and/or lower limit values to the meaningful attribute data being monitored, e.g. frequency, amplitude, high-temperature threshold, low-temperature threshold, or another parameter, condition alarms/alerts can be generated when the data exceeds or drops below a limit. This condition might be indicative of a worsening of, for example, bearing wear which could lead to an imminent equipment failure.

The value of being able to closely monitor the state of, for example, vibration in this example enables either a user or the system to take evasive or corrective action, such as dispatching a service technician, shutting down the equipment, or lowering engine RPM, thereby averting a potential costly failure. The cost avoidance is not only associated with the cost of replacing all or part of the equipment, but also the value of the lost production during equipment downtime during repairs. Further, when the improper vibration location is identified, the service technician has a starting point from which to investigate potential part replacement and/or repair. Furthermore, if the data, such as vibrational data, is gathered at frequent intervals (for example, every 5-60 seconds), the RPM of the machine, such as an engine, can be plotted to better understand the operation performance of that particular engine as a function of time. Coupled with other engine variables, such as individual cylinder-based engine exhaust temperature, the collected data can be studied to correlate the relationship between the variables for each engine and a database can be established for hundreds or thousands of engines, which can serve as a predictive tool for measurements received from other engines. The same is true for machines or devices other than engines.

The shorter the interval between data measurements (frequent measurements), the more likely the plotted data can be used to predict behavior that may lead to impending equipment failure. By creating a histogram (charted data values over time) of the collected data, and applying data trend modeling algorithms, systems and methods of the invention can predict certain characteristics that could lead to imminent failure if left unchecked, such as bearing seizer leading to a broken or bent valve.

A device or system according to the invention has the ability to enter into a learning mode by plotting data over time in order to establish the standard operating parameters of the machine. To initiate the learning mode, the device or system is activated to capture data from the machine over a specified time period (e.g., 10 seconds-60 minutes). The captured data can then be analyzed to determine the normal operating condition of a particular machine or device.

The learning mode, which is preferably part of the normal operation of a system or method according to embodiments of the invention, can be engaged under a variety of situations such as one or more of: during startup, half-normal operating speed (e.g., 600 RPM for an engine), full-operating speed (e.g., 1,200 RPM for an engine) with no load, or operating speed with various load states. When in the learning mode the device or system records and calibrates parameters such as temperatures and vibrational signals under proper working parameters for baseline measurements. Calibration establishes upper and lower calibration settings, which form the standard operating parameter foundries. Ambient environmental factors also can be recorded as part of the data set, which can be calculated into the standard operating parameters. The standard operating parameters may be unique for each machine and for each cylinder (if the machine has cylinders).

In one embodiment, the data is collected and transmitted to an intermediary device called a coordinator, which then transmits the data to a gateway, and or another repository via wireless or wired communications.

Computational analysis can be performed by the device or system, such as by an integrated microprocessor that may be integral to the device, the device or system, such as by the coordinator, the gateway or another part of the system.

The analysis may include identifying the standard operating parameters (“SOP”) and comparing the newly measured data to the SOP to ascertain whether an intervention or escalation procedure, or preemptive or preventative maintenance should be undertaken. The learning mode is preferably re-conducted after any engine transport and/or significant mechanical work (e.g., upper valve train overhaul) is conducted on the machine in order to re-calibrate and establish the SOP.

In another embodiment, currently, in many cases, a gas flow meter is used in the downstream leg of a compressor to determine the flow of gas being delivered by the compressor. The accuracy of this meter is based upon proper calibration and upkeep of the system. Often, the entities supplying equipment to pump the natural gas are paid based upon the amount of gas pumped. Therefore, the economic value of the natural gas being gathered, compressed and delivered for distribution is dependent on the accuracy of the EFMs. Gas losses due to leaks not attributable to pumping equipment and errors in EFM calibration can lead to a loss of revenue. Devices and methods according to aspects of the invention can accurately measure the amount of gas being delivered.

In the case when the machine is a multi-cylinder, reciprocating gas compressor, which is typically used in the midstream natural gas gathering compression industry, the ability to detect the RPM of the rotating crankshaft can be used to determine the volumetric flow rate of gas through the compressor. This ability can be useful in determining the production value (i.e., the cfm/hr and $/hr) of the natural gas processed by the compressor and delivered to the distribution pipeline. As one example, given the following values: (1) the compressor RPM (calculated by a sensor in communication with an accelerometer), (2) the number of cylinders, (3) the cylinder bore diameter, (4) the piston stroke length, and (5) the inlet gas pressure; the total volumetric and mass flow rate of the gas being delivered by the compressor can be calculated. Hence, use of a system or device of the invention, outfitted with accelerometer sensor or similar apparatus, can be used to determine the volumetric throughput of a gas compressor.

Another method of determining the volumetric flow rate through a compressor is to reference a look-up table (stored in a memory, which may be on a PCB-mounted microprocessor) that contains the flow rate data from the compressor manufacturer. When a sensor according to the invention determines the compressor RPM, this measured value can be processed, such as by a microprocessor, to obtain the flow data from a library of flow-data values provided by the manufacturer. This data resides in a database that can be accessed by the microprocessor. As an example, per a manufacturer's (such as Dresser-Rand) specifications, a reciprocating compressor having a 9.0″ diameter cylinder, with a piston stroke of 7.25″, running at 1,000 RPMs, should displace 847 cubic meters per hour (m3/hr) of gas per cylinder. If the compressor was a 6 cylinder unit, the total volumetric flow rate would be 5,082 m3/hr (847×6).

In accordance with various embodiments, a volatile organic compound (VOC) sensor device can comprise a sensor located in proximity to a tank vent of a storage tank, wherein the sensor can be configured to monitor flumes from the tank vent; a controller operatively coupled to the sensor, wherein the controller can be configured to receive a measured input from the sensor, wherein the measured input can be VOC measurement data of the flumes; and a wireless communication device coupled to the controller, wherein the wireless communication device can be configured to communicate with a coordinator.

Furthermore, in various embodiments, a method of volatile organic compound (VOC) monitoring can comprise monitoring, by a sensor located in proximity to a tank vent of a storage tank, flumes from the tank vent; receiving, by a controller operatively coupled to the sensor, a measured input from the sensor, wherein the measured input can be VOC measurement data of the flumes; communicating, by a wireless communication device coupled to the controller, with a coordinator.

In accordance with various embodiments, an air monitoring array system can comprise a plurality of air quality sensor devices arranged within a selected area, which can be configured to measure air pollutant levels in the selected area. Furthermore, each of the plurality of air quality sensor devices can comprise at least one sensor operatively coupled to a controller, and a wireless communication device also coupled to the controller. In various embodiments, the controller can be configured to receive a measured input from the at least one sensor. Also, the wireless communication device can be configured to communicate with a central server.

In accordance with various embodiments, a method of air quality monitoring can comprise measuring, by a plurality of air quality sensor devices arranged within a selected area, air pollutant levels in the selected area. Each of the plurality of air quality sensor devices can comprise at least one sensor operatively coupled to a controller, wherein the controller can be configured to receive a measured input from the at least one sensor; and a wireless communication device coupled to the controller, wherein the wireless communication device can be configured to communicate with a central server.

In accordance with various embodiments, a selective holding tank draining system can comprise a sensor device configured to receive total dissolved solids (TDS) data of a stored fluid from a TDS sensor, and wherein the sensor device can be configured to receive volume data of the stored fluid from a volume sensor, and a central server configured to determine a selected TDS level for disposal of the stored fluid. In various embodiments, an average TDS level of a drained volume of the stored fluid if draining from two or more tanks can be calculated. Furthermore, the stored fluid volume to drain from each of the two or more tanks to achieve a drained mixture having less than the selected TDS level can be determined.

In various embodiments, a method of selective holding tank draining can comprise receiving, by a sensor device, TDS data of a stored fluid from a TDS sensor; receiving, by the sensor device, volume data of the stored fluid from a volume sensor; determining, by a central server, a selected TDS level for disposal of the stored fluid; calculating an average TDS level of a drained volume of the stored fluid if draining from two or more tanks; and determining a stored fluid volume to drain from each of the two or more tanks to achieve a drained mixture have less than the selected TDS level.

In accordance with various embodiments, a quality monitoring method can include receiving, by a sensor device, total dissolved solids (TDS) data of a stored fluid from a TDS sensor in real-time; transmitting, by the sensor device, the TDS data to a coordinator; and comparing the TDS data to a TDS threshold level. A quality monitoring system can comprise a sensor device configured to receive total dissolved solids (TDS) data of a stored fluid from a TDS sensor, and a coordinator configured to receive the TDS data from the sensor device.

In accordance with various embodiments, a sensor device can comprise at least one sensor operatively coupled to a controller, wherein the controller is configured to receive a measured input from the at least one sensor; and a wireless communication device coupled to the controller. Further, the wireless communication device can be configured to communicate with a coordinator. In various embodiments, the at least one sensor can include a volume sensor, a flow meter sensor, a total dissolved solids sensor, an infrared thermal monitor, an air quality sensor, or any combination thereof.

In accordance with various embodiments, a holding tank monitoring system can include a sensor device configured to receive total dissolved solids (TDS) data of a stored fluid from a TDS sensor in real-time. The TDS sensor can be located near an input of a holding tank storing the stored fluid. In addition, the TDS sensor data can be used to determine water production of a natural resource well. For example, predictive analysis can be used to determine expected remaining production of the well based in part on the water production. Moreover, a holding tank monitoring method can include receiving, by a sensor device, total dissolved solids (TDS) data of a stored fluid from a TDS sensor in real-time, determining water production of a natural resource well based on the TDS sensor data, and determining expected remaining production of the well using predictive analysis based in part on the water production.

In accordance with various embodiments, a logistics system can comprise a plurality of sensor devices providing data, a capacity module, an identification module, and a processor. Each of the plurality of sensor devices can be in communication with an individual holding tank. Further, the data can include flow rate of the individual holding tanks, and where the data identifies the individual holding tank locations. The capacity module can be configured to determine the time remaining until each of the individual holding tanks reaches capacity based on the flow rate and remaining capacity of the individual holding tanks. In addition, the identification module can be configured to identify a fleet of tanker trucks for draining the individual holding tanks Moreover, the processor can implement a mathematical model populated by the data, where the mathematical model can comprise an objective function for minimizing tanker truck driven miles and preventing the individual holding tanks from reaching capacity.

Furthermore, in various embodiments, a logistics method can comprise receiving data from a plurality of sensor devices, wherein each of the plurality of sensor devices can be in communication with an individual holding tank, and wherein the data can comprise a flow rate of the individual holding tanks, and wherein the data identifies the individual holding tank locations; determining a remaining time period until each of the individual holding tanks reaches capacity based on the flow rate and a remaining capacity of the individual holding tanks; identifying a fleet of tanker trucks for draining the individual holding tanks; and using the data to populate a mathematical model that can comprise an objective function for minimizing tanker truck driven miles and preventing the individual holding tanks from reaching capacity.

### BRIEF DESCRIPTION OF THE DRAWINGS

A more complete understanding of the embodiments of the present disclosure may be derived by referring to the detailed description and claims when considered in connection with the following illustrative figures.

FIG. 1 illustrates an exemplary embodiment of a system according to various aspects of the invention.

FIG. 2 depicts an exemplary sensor device in accordance with various aspects of the invention.

FIG. 3 illustrates another exemplary system according to aspects of the invention.

FIG. 4 is an exploded view of a casing of a device in accordance with an aspect of the invention.

FIG. 4A is a device according to aspects of the invention mounted on the valve cover of an engine.

FIG. 5 is an exploded view of a device according to an aspect of the invention.

FIG. 6 is a cross-sectional view of the device of FIG. 5 assembled and mounted on a valve cover of an engine.

FIG. 7 is a perspective, top view of the assembled device of FIG. 5 mounted on a valve cover of an engine.

FIG. 7A is a perspective view of the device of FIG. 7 mounted on an engine.

FIG. 8 is a bottom view of a device in accordance with an aspect of the invention.

FIG. 9 is the device of FIG. 8 with two mounting legs attached.

FIG. 10 is a cross-sectional view of a device according to an aspect of the invention.

FIG. 11 shows the device of FIG. 10 mounted on a valve cover of an engine.

FIG. 12 shows a side view of the device of FIG. 5.

FIG. 13 is a side view of an engine on which a system, device, or method according to the invention may be utilized.

FIG. 14 is a close-up view of the valve covers on one side of the engine of FIG. 13.

FIG. 15 depicts an engine according to FIGS. 13 and 14 including devices according to an aspect of the invention and depicts the devices communicating data received from the engine.

FIG. 16 depicts a plurality of tank farms utilizing a system according to an aspect of the invention.

FIG. 17 shows an enlarged device according to aspects of the invention.

FIG. 18 shows a comparison of the device of FIG. 17 to a device designed to power the measuring of operational data for a single engine cylinder.

FIGS. 19A-19B depict the device of FIG. 18.

FIG. 20 depicts a partial cross-sectional view of the device of FIG. 18.

FIG. 21 illustrates an exemplary embodiment of a sensor system and communications according to various aspects of the invention;

FIG. 22 illustrates an exemplary communication system of sensor devices in accordance with various aspects of the invention;

FIG. 23 illustrates an exemplary embodiment of an air quality monitoring system in accordance with various aspects of the invention;

FIG. 24 illustrates an exemplary embodiment of a truck routing system in accordance with various aspects of the invention;

FIG. 25 illustrates an exemplary method of predicting a natural resource well lifespan in accordance with various aspects of the invention;

FIG. 26 illustrates an exemplary method of monitoring total dissolved solid levels in accordance with various aspects of the invention;

FIG. 27 illustrates an exemplary method of selective storage tank drain mixtures in accordance with various aspects of the invention;

FIG. 28 illustrates an exemplary method of monitoring volatile organic compounds in accordance with various aspects of the invention;

FIG. 29 illustrates an exemplary method of determining truck routing logistics based on remote asset monitoring in accordance with various aspects of the invention.

FIG. 30 depicts a pipe section including a vibrational measurement device.

### DETAILED DESCRIPTION OF EXEMPLARY EMBODIMENTS

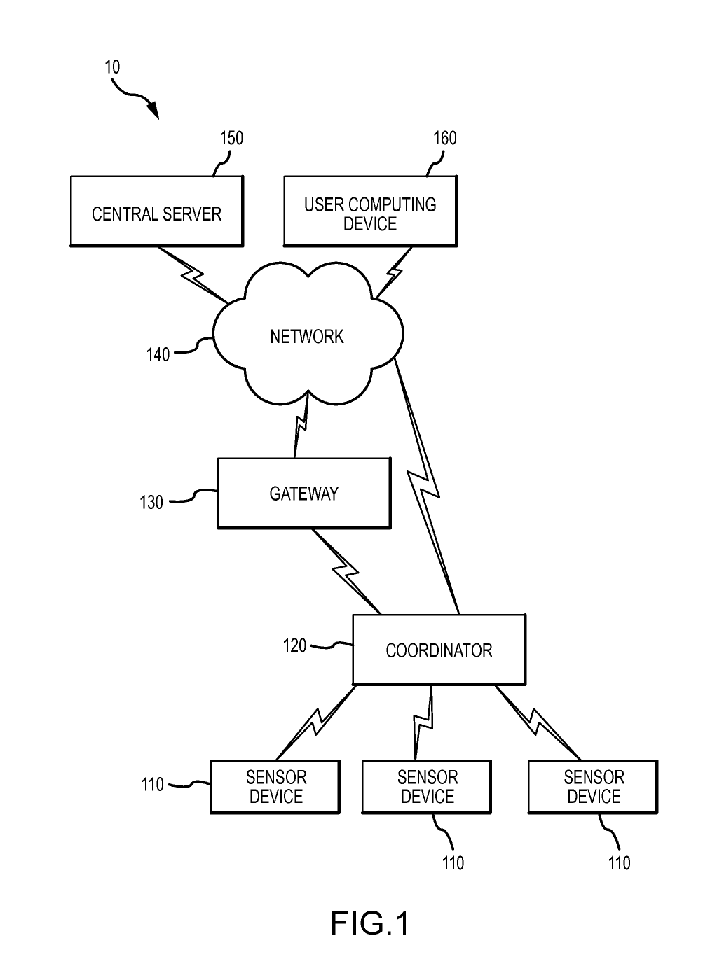

Turning now to the Figures, where the purpose is to describe exemplary embodiments of the invention and not to limit same, an exemplary system according to various aspects of the present invention is depicted in FIG. 1. The system 10 includes one or more sensor devices 110 preferably communicating with a coordinator 120. The coordinator 120 preferably communicates with central server 150 and user computing device 160 via gateway 130 and/or network 140 or through any suitable method or communications device. Sensor devices 110 are sometimes referred to herein as “motes,” and coordinators 120 are sometimes referred to herein as “nodes.” The functionality of sensor device 110, coordinator 120, server 150, computing device 160, gateway 130 and/or any other component operating in conjunction with aspects of the present invention can be implemented in any suitable manner, such as through a processor executing software instructions stored in a memory. Functionality may also be implemented through various hardware components storing machine-readable instructions, such as application-specific integrated circuits (ASICs), field-programmable gate arrays (FPGAs) and/or complex programmable logic devices (CPLDs).

Sensor Device 110

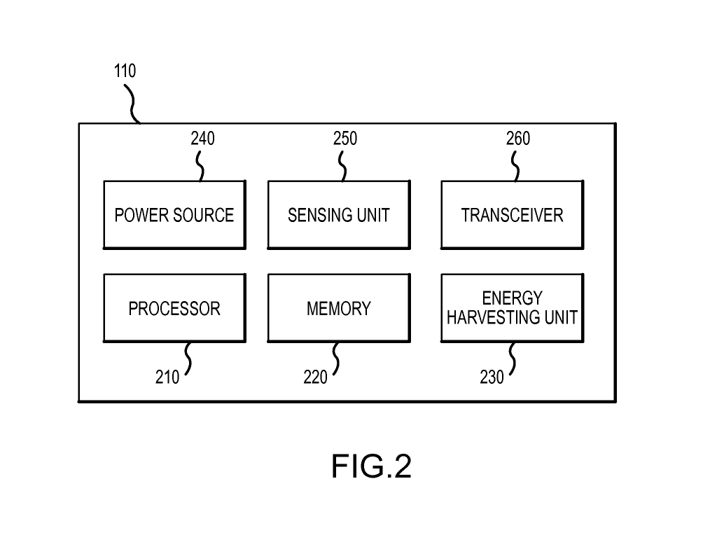

The sensor device(s) 110 collect information regarding one or more assets being monitored. Embodiments of the present invention may operate in conjunction with any number and type of sensor devices. An exemplary sensor device 110 is depicted in block diagram form in FIG. 2. In this exemplary embodiment, sensor device 110 includes a processor 210, memory 220, energy harvesting unit 230, power source 240, sensing unit 250, and transceiver 260. As used herein, a “sensing unit” refers to any type of sensor, while a “sensor device” refers to any system or device capable of receiving data from one or more sensing units. As an example, a sensing unit may measure vibration, temperature or any operational parameter, and the data is then received by a sensing unit, or sensor device, 110.

Processor 210

In the exemplary sensor device 110 depicted in FIG. 2, the processor 210 retrieves and executes instructions stored in the memory 220 to control the operation of the sensor device 110. Any number and type of processor(s) such as an integrated circuit microprocessor, microcontroller, and/or digital signal processor (DSP), can be used in conjunction with the present invention. The processor 210 may include, or operate in conjunction with, any other suitable components and features, such as comparators, analog-to-digital converters (ADCs), and/or digital-to-analog converters (DACs).

Memory 220

The exemplary sensor device 110 depicted in FIG. 2 includes a memory 220 capable of storing executable instructions, data, messages transmitted to or received from other components of system 100, and other information. A memory 220 operating in conjunction with the present invention may include any combination of different memory storage devices, such as hard drives, random access memory (RAM), read only memory (ROM), FLASH memory, or any other type of volatile and/or nonvolatile memory.

Energy Harvesting Unit 230

The energy harvesting unit 230 collects energy to supply power to, or recharge, the power source 240. In some embodiments, the energy harvesting unit 230 may power the sensor device 110 directly. The energy harvesting unit 230 may include a photovoltaic cell for collecting solar energy; a thermoelectric generator (TEG); and/or a piezoelectric vibrational energy harvester (PZEH). In some exemplary embodiments, a TEG and/or PZEH is used to generate energy from the heat (or vibration, respectively) generated by an asset such as an engine or compressor being monitored. In this manner, the operation of the asset itself can provide some or all of the power necessary to monitor the asset using the sensor device 110. Embodiments of the invention may include multiple energy harvesting units 230 to provide for additional (or redundant) power generation.

Power Source 240

The power source 240 powers the various components of the sensor device 110. The exemplary sensor device 110 depicted in FIG. 2 is powered by a solid-state Li-PON battery, though any number, combination, and type of suitable power sources can be utilized in embodiments of the present invention. In the exemplary sensor device 110 depicted in FIG. 2, the Li-PON battery is rechargeable via the energy harvesting unit 230, and may also be charged through a dedicated power connector, if desired.

Sensor Unit 250

The sensor unit 250 measures characteristics related to an asset. The sensor unit 250 may be configured to measure any number of desired characteristics, such as temperature, pressure, flow, vibration, strain, electrical parameters (such as voltage, resistance, and current), atmospheric characteristics (such as moisture and gas content), sound, a chemical, radiation, position, force, movement, and/or any other measurable characteristic.

Some engines, compressors, and other assets may include built-in sensor networks for monitoring various aspects of the operation of the asset. While embodiments of the invention need not rely on these built-in sensor networks to monitor an asset, some embodiments may be configured to receive the data from such networks. Embodiments of the invention can thus fully monitor assets without built-in sensor networks (or where the data from such networks is restricted, encoded, etc.) while utilizing data from such networks if/when such data is available.

Information provided by the sensor unit 250 may be formatted as desired. For example, analog data regarding vibrations of a monitored internal combustion engine may be converted (using an analog to digital converter, for example) to a digital format, and subsequently formatted into a data packet including a data header followed by one or more data values. Similarly, the sensor device 110 may store a series of measurements from multiple sensor units 250 in the form of a spreadsheet with headers indicating the source of the measurements. Such spreadsheets can be transmitted remotely via network 140 to server 150, or accessed locally by a technician via a mobile device 310 and a local wireless network.

Transceiver 260

The transceiver 230 communicates with one or more other systems, such as the coordinator 120, gateway 130, network 140, and/or any other suitable systems. Any suitable communications device, component, system, and method may be used in conjunction with the transceiver 260. In some exemplary embodiments, the transceiver 260 comprises a Bluetooth transceiver configured to communicate with a coordinator 120.

The sensor device 210 may include, or operate in conjunction with, any type and number of transceivers 260. In some embodiments, the sensor device 110 includes a cellular radio frequency (RF) transceiver and may be configured to communicate using any number and type of cellular protocols, such as General Packet Radio Service (GPRS), Global System for Mobile Communications (GSM), Enhanced Data rates for GSM Evolution (EDGE), Personal Communication Service (PCS), Advanced Mobile Phone System (AMPS), Code Division Multiple Access (CDMA), Wideband CDMA (W-CDMA), Time Division-Synchronous CDMA (TD-SCDMA), Universal Mobile Telecommunications System (UMTS), and/or Time Division Multiple Access (TDMA). The transceiver 260 may communicate using any other wireless protocols, such as a Zigbee protocol, a Wibree protocol, an IEEE 802.11 protocol, an IEEE 802.15 protocol, an IEEE 802.16 protocol, an Ultra-Wideband (UWB) protocol, an Infrared Data Association (IrDA) protocol, a Bluetooth protocol, and combinations thereof.

A sensor device 110 operating in conjunction with the present invention may alternatively (or additionally) communicate using any other method of wired or wireless communication. For example, in some embodiments the transceiver 260 may be configured to communicate using one or more wired connections using, without limitation: tip and sleeve (TS), tip, ring, and sleeve (TRS), and tip, ring, ring, and sleeve (TRRS) connections; serial peripheral interface bus (SPI) connections; universal serial bus (USB) connections; RS-232 serial connections, Ethernet connections, optical fiber connections, and Firewire connections. The transceiver 260 can be configured (e.g. through a software program residing in memory 220 and executed by processor 210) to detect and switch to different communication protocols and/or different wired or wireless connections, thus allowing communications with a wide variety of devices.

The sensor device 110 may be configured to detect, analyze and/or transmit data from any number of different sensor units 250 in which it is in communication. Additionally, the sensor device 110 may be configured to perform any desired analysis of the data from the sensor units 250, including those described below. In various embodiments, individual sensor units 110 may be configured to detect a potential problem associated with a monitored asset.

Coordinator 120

The coordinator 120 preferably communicates with one or more sensor devices 110. The coordinator 120 may be configured to communicate using any desired wired or wireless communication connection or protocol, including those described above. In some embodiments, the coordinator 120 is configured to communicate with a plurality of sensor devices 110 and, in turn, communicate with other coordinators 120, with gateway 130, and/or with other systems (such as server 150) via the network 140. In this manner, a single coordinator can communicate with multiple sensor devices 110 using a short-range, low-power communication protocol (e.g., Bluetooth) and communicate with other systems (such as gateway 130) using a longer-range protocol, resulting in less overall power consumption by embodiments of the invention.

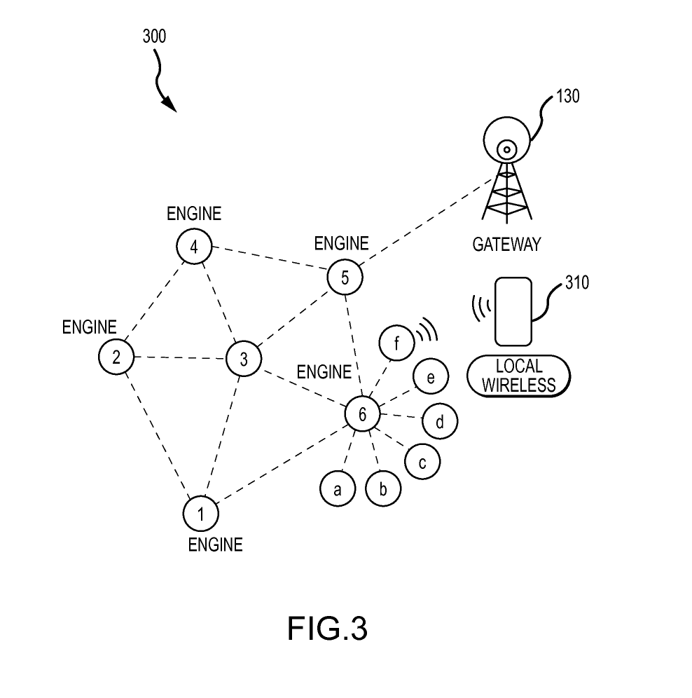

Referring now to FIG. 3, a network of coordinators 120 (labeled 1-6) is shown, with each coordinator corresponding to a respective engine being monitored. As shown for coordinator 6, each coordinator (1-6) communicates with a respective plurality of sensor devices 110 (labeled a-f with respect to coordinator 6) via short-range wireless protocol (Bluetooth in this example). In this exemplary embodiment, coordinators 1-6 communicate with each other and/or with gateway 130 using a longer-range wireless protocol (an 802.15 protocol in this example) and adjacent coordinators are no more than about 300 feet from each other. At this range, the coordinators can communicate with immediately adjacent coordinators (shown by the dotted lines between coordinators), but only coordinator 5 is within range of gateway 130. In such cases, coordinators operating in conjunction with embodiments of the invention may be configured to relay communications so that all coordinators can communicate with or through the gateway 130.

For example, coordinator 4 may transmit data to coordinator 5 for rebroadcast to gateway 130. Likewise, coordinator 1 may transmit data to gateway 130 through coordinators 3 and 5. In some embodiments, communications can be alternately relayed through different coordinator nodes to help avoid over-burdening any one particular node. For example, coordinator 1 may first communicate with gateway 130 via coordinators 6 and 5, and next communicate with gateway via nodes 3 and 5.

As also shown in FIG. 3, sensor devices 110 (labeled a-f in FIG. 3) can communicate with local device(s) 310. This allows, among other things, technicians to communicate directly with sensing devices 110 (to perform diagnostics or other functions) without having to access network 140 or server 150.

Gateway 130

The gateway 130 communicates with coordinator 120 and with other systems (such as central server 150 and user computing device 160) via network 140. In some embodiments, such as in the exemplary system 300 depicted in FIG. 3, gateway 130 is disposed within communication range of at least one coordinator 120. In some embodiments, gateway 130 communicates with one or more coordinators 120 using a first wireless communication protocol (e.g., an 802.15 protocol) and communicates with network 140 using a second wired or wireless communication protocol (e.g., a longer-range protocol such as a cellular protocol), including those described previously. Among other things, the gateway 130 helps maximize the efficiency of the overall power consumption of system 300 and other embodiments of the invention by using short range (and lower power) communication protocols between sensor devices 110 and coordinators 120 and a longer range protocol to communicate with remote devices via network 140.

In the exemplary embodiments depicted in FIGS. 1 and 3, gateway 130 includes multiple transceivers to communicate (simultaneously if desired) using different communication protocols, thus allowing the gateway to, for example, communicate with a coordinator 120 and central server 150 via network 140 at the same time. The gateway 130 may also be configured to store and process information collected from the sensors 110. The gateway 130 can thereby provide a technician with local access to data accessible via a mobile computing device 310 and retain a copy of data in case of a hardware or communication failure related to server 150.

While coordinator 120, gateway 130, and network 140 are shown as separate components in FIG. 1, alternate embodiments may perform the functionality of these components using a single system or device. Additionally, some embodiments may use more or fewer components to collect data from the sensor devices 110.

Network 140

The network 140 allows the sensor devices 110, coordinator 120 and/or gateway 130 to communicate with other systems and devices, such as central server 150 and user computing device 160. The network 140 may include any combination of wired and wireless connections and protocols, such as those described above. The network 140 may comprise a local area network (LAN), wide area network (WAN), wireless mobile telephony network, General Packet Radio Service (GPRS) network, wireless Local Area Network (WLAN), Global System for Mobile Communications (GSM) network, Personal Communication Service (PCS) network, Advanced Mobile Phone System (AMPS) network, and/or a satellite communication network. In some embodiments, network 140 includes the Internet to allow the central server 150 or computing device 160 to communicate with sensor devices 110, coordinator 120 and/or gateway 130 from anywhere an Internet connection can be established. As such, embodiments of the invention provide efficient, centralized monitoring of assets even in applications (such as oil and gas production) where monitored assets are in remote locations and often spread across large areas.

Central Server 150

In the exemplary embodiment depicted in FIG. 1, the central server 150 receives and analyzes data from the sensor devices 110 and can issue commands to control sensor device 110, coordinator 120, gateway 130, and/or an asset being monitored.

The central server 150 may receive data from the sensor devices 110 in any desired manner. In some embodiments, the server 150 is configured to automatically request data from one or more of the sensor devices 110 via the network 140, gateway 130, and coordinator 120. Alternatively, the sensor device 110, coordinator 120, gateway 130, or any other device operating in conjunction with embodiments of the invention can be configured to automatically request and/or transmit data in any suitable manner. For example, each sensor device 110 may be configured to collect and send data measured from a monitored asset (such as an internal combustion engine or compressor) and automatically transmit such data to the coordinator 120 at periodic intervals (e.g., every 15 seconds). The coordinator 120, in turn, may immediately retransmit the data to the server 150 via network 140 and/or to gateway 130, or may store the data for analysis and/or later transmittal.

The transmission of data by a device operating in conjunction with the present invention may be subject to any suitable conditions or rules that dictate whether the data is transmitted. For example, a device may first check to verify (1) that a device designated to receive the data is within range; (2) that both devices have sufficient battery reserves to send the request and receive the data; (3) that the receiving device has sufficient space in its memory to store the data, and/or whether any other suitable condition is met.

User access to the server 150 may be controlled via an authentication process. In some embodiments, authentication is authorized using authentication tokens. In various embodiments, authentication tokens may comprise either simple or complex text strings or data values indicating an account number or other user identifier that can be matched against an internal database by the central server 150. Alternatively, authentication tokens may comprise encoded passwords or other indicia that assert that the entity for whom authentication is requested is genuine. Generation of an authentication token may be accomplished using alternative methods such as entry of a user identifier, PIN, or password by the user after being prompted to do so. Alternatively, a biometric measurement of the user could be obtained and the measurement rendered into a digital representation. Once generated, for security purposes the authorization token may be secured by encrypting the token, digesting and encrypting the digest of the token, or cryptographically hashing the token before transmission to the requesting entity. When authentication tokens are created, the originating component of the token may create a certification of validity through at least one of the following methods: (1) encrypting the token with a private key associated with the token originator; (2) encrypting the token with a public key associated with the token requester or destination; (3) generating a digest of the token (through a method such as a hashing algorithm discussed above) and optionally encrypting the hashed digest with the token originator's private key, or (4) providing an authentication code as at least part of the token (such as a cryptographically hashed password) that may be is compared to previously stored values. When a component receives the token along with any encrypted or cleartext certification data, the component may determine the access is valid by (1) attempting to decrypt an encrypted token with the alleged originator's public key; (2) attempting to decrypt an encrypted token with the alleged originator's public key; (3) attempting to decrypt an encrypted digest with the alleged originator's public key, and comparing the result to a hashed value of the token, pin, code, or password, or (4) comparing a cryptographically hashed password for the alleged originator to known pre-stored values, and if a match is found, authorization is granted.

User Computing Device 160

A user computing device 160 can communicate with any of the other components in system 100. The user computing device 160 may include a personal computer or a mobile computing device, such as a laptop computer, a mobile wireless telephone, or a personal digital assistant (PDA).

A user can use computing device 160 to view, in real-time or near-real-time, the status of any of the components of a system of the present invention, such as the components shown in the Figures. The computing device 160 may also be used to send commands to control such components or to the monitored asset, as well as to view reports showing data from the sensor devices 110, or to analyze the data to generate metrics regarding the status of the monitored asset. Data can be provided to or received from a user of the computing device 160 in a machine-readable format. The computing device 160 may be configured to send, receive, and process machine-readable data can in any standard format (such as a MS Word document, Adobe PDF file, ASCII text file, JPEG, or other standard format) as well as any proprietary format. Machine-readable data to or from the user interface may also be encrypted to protect the data from unintended recipients and/or improper use.

The server 150 or user computing device 160 may include any number and type of processors to retrieve and execute instructions stored in the memory storage device of the server to control its functionality. The server 150 may include any type of conventional computer, computer system, computer network, computer workstation, minicomputer, mainframe computer, or computer processor, such as an integrated circuit microprocessor or microcontroller in accordance with the present invention. The server 150 or computing device 160 operating in conjunction with the present invention may include any combination of different memory storage devices, such as hard drives, random access memory (RAM), read only memory (ROM), FLASH memory, or any other type of volatile and/or nonvolatile memory. The server 150 may include an operating system (e.g., Windows, OS2, UNIX, Linux, Solaris, MacOS, etc.) as well as various conventional support software and drivers typically associated with computers. Software applications stored in the memory may be entirely or partially served or executed by the processor(s) in performing methods or processes of the present invention.

The server 150 or computing device 160 may also include a user interface for receiving and providing data to one or more users. The user interface may include any number of input devices such as a keyboard, mouse, touch pad, touch screen, alphanumeric keypad, voice recognition system, or other input device to allow a user to provide instructions and information to other components in a system of the present invention. Similarly, the user interface may include any number of suitable output devices, such as a monitor, speaker, printer, or other device for providing information to one or more users.

Any of the components can be configured to communicate with each other (or with other additional systems and devices) for any desired purpose. For example, the server 150 or user computing device 160 may be used to upload software to sensor device 110 or other component, provide or update encryption keys, and to perform diagnostics on any of the components in systems 100 or 300. Any computer system may be configured (i.e., using appropriate security protocols) to communicate instructions, software upgrades, data, and other information with components via network 140. In some embodiments, data received from the sensor devices 110 is processed into a report and electronically provided (i.e., via email) to multiple users in a ubiquitous data format such as Portable Document Format (PDF). Such reports can be created at the request of a user or generated automatically at predetermined times or in response to the occurrence of an event (such as a detected problem with a monitored asset).

Any combination and/or subset of the elements of the methods depicted herein may be practiced in any suitable order and in conjunction with any system, device, and/or process. The method described herein can be implemented in any suitable manner, such as through software operating on one or more systems or devices, including the systems 100 or 300.

Collecting Data From Sensor Devices

As described above, the sensor devices 110 may include, or connect to, any type of sensor. In some embodiments, sensor devices 110 are coupled to accelerometers, which are deployed to monitor the vibration(s) of an internal combustion engine or compressor used in the production or transport of oil or gas. The sensor devices 110 and sensors may be strategically positioned to monitor different sources of vibration on an engine, such its valves, crankshaft, or bearings.

Transmit Data

Data collected from a sensor device 110 or generated by any other device operating in conjunction with the present invention may be transmitted to other systems, such as to central server 150 for analysis. The data can be transmitted in any suitable manner, including using any of the wired or wireless communication methods and protocols described previously. Any amount of data can be transmitted in any manner. For example, data from the sensor device 110 can be transmitted to another device (such as to coordinator 120) as it is measured, or data can be stored (such as in a memory storage device in the sensor device 110) for a period of time before being transmitted to another device. In some cases, for example, it may be more efficient to transmit blocks of data at once rather than initiating communication with another device each time data is available. In other cases, a device may be out of range or otherwise unavailable to receive the data. The data can also be stored for any desired length of time, and/or until a particular event occurs. For example, the device data could be stored until it is verified that the receiving device and/or the data server 150 have received the data, allowing the data to be retransmitted if necessary. Data can also be deleted when a data record exceeds a predetermined storage time, and/or the oldest data record is deleted first after a predetermined storage size limit has been reached.

Data transmitted from the sensor devices 110 may be validated to ensure it was transmitted properly and completely. The sensor device data may also be validated to ensure it was provided from a specific sensor device 110 or group of sensor devices 110 (i.e., associated with a particular asset being monitored). The data may also be validated to ensure that fields in the data correspond to predetermined values and/or are within certain thresholds or tolerances. Any number, code, value or identifier can be used in conjunction with validating the device data. For example, the data can be validated by analyzing a serial number, a device identifier, one or more parity bits, a cyclic redundancy checking code, an error correction code, and/or any other suitable feature.

In exemplary embodiments of the present invention, various components (such as coordinator 120, gateway 130, and server 150) may be configured to receive data directly or indirectly from a sensor device 150, format a message based on the data, and transmit the formatted message to another system or device. This functionality may be implemented through software operating on any suitable mobile computing device and with any computer operating system.

Receipt of data from the sensor devices 110 may be restricted only to authenticated devices operating as part of the present invention. Authentication can also prevent sensitive data from being broadcast and viewed by unintended recipients. Any device may be authenticated to verify the device is able to receive, process, and/or transmit data. During authentication, the authenticated device or devices may also be remotely commanded, and such commands may include steps that configure devices to interoperate with components of the present invention. For example, but not by way of limitation, such steps may include the downloading of software applications, applets, embedded operating code, and/or data.

Devices can be authenticated in any manner. For example, devices can be authorized to receive data from one or more sensor devices 110 using an authorization code. The authorization code can be any number, code, value or identifier to allow the receiving device to be identified as a valid recipient of the data. In some embodiments, the receiving device stores an authorization code and broadcasts the authorization code in response to a request for authorization. Unless the authorization code matches a code stored by the transmitter of the data (such as the sensor device 110 itself or another transmission device), the data is not transmitted to the device.

In other exemplary embodiments of the present invention, the coordinator 120, gateway 130, or other device receiving the data from the sensor device 110 using a wireless network protocol (such as Bluetooth) is authenticated based on whether the receiving device advertises one or more services. In this context, advertised services reflect functions, utilities, and processes the receiving device is capable of performing. The receiving device broadcasts indicators of this functionality, thus “advertising” them to other systems and devices. In such embodiments, unless the receiving device advertises a service that is identifiable with the operation of the present invention (i.e., a process capable of broadcasting the sensor device 110 data to the central server 150, for example), the receiving device is not authenticated and thus the data is not transmitted to the device.

Data can be transmitted to components operating in conjunction with the present invention in any format. For example, data from the sensor device 110 can be transmitted to the coordinator 120 exactly as it is generated by the sensing unit 250 of the sensor device 110, or it can be reformatted, modified, combined with other data, or processed in any other suitable manner before being transmitted. For example, the data can be encrypted prior to transmission, and this encryption may occur at any stage in its transmission by the sensor device 110 or retransmission by another device. Some or all of the data being transmitted may be encrypted. In some embodiments, a digest of the data may be encrypted, to digitally “sign” the data contents to verify its authenticity. For example, but not by way of limitation, this digest may be produced by providing the received data to a hashing algorithm such as the MD5 or SHA-1 Secure Hashing Algorithm as specified in National Institute of Standards and Technology Federal Information Processing Standard Publication Number 180-1.

Asymmetric encryption algorithms and techniques are well known in the art. See, for example, RSA & Public Key Cryptography, by Richard A. Mollin, CRC Press, 2002, and U.S. Pat. No. 4,405,829, issued Sep. 20, 1983, the disclosures of which are incorporated herein by reference. In an illustrative example, if two parties (for example, “Alice” and “Bob”) wish to communicate securely using public key cryptography, each party begins by generating a unique key pair, where one of the keys is a private key that is kept in confidence by that party, and the other key is a public key that may be publicly distributed, published only to a message recipient, or made available through a public key infrastructure. The key generation step need be done by a party only once, provided that the party's private key does not become compromised or known by another party. If Alice wants to send a message confidentially to Bob, she may use Bob's public key to encrypt the message, and once sent, only Bob can decrypt and view the message using Bob's private key. But if Alice also wanted Bob to have assurance that the message was in fact coming from her, she could further encrypt the message with her private key before sending, then when Bob's private key and Alice's public key are used to decrypt the message, Bob knows for certain that he was the intended recipient and that Alice was the one who originated the message, and Alice knows that only Bob will be able to decrypt and read her message.

Asymmetric cryptography may be utilized to enhance security of certain implementations of the present invention. In some embodiments, data transmitted by a sensor device 110 is encrypted with a private key, or with a public key of the intended recipient system (such as the coordinator 120), or with both keys. The private and/or public keys may be delivered to a receiving device through a wired or wireless connection, allowing the receiving device to be configured for secure operation. In some embodiments, the server 150 may request that the public key of a sensor device 110 be forwarded to enable decryption of any information encoded with the user's private key. In this manner, the data may be authenticated as coming from the actual asset that is desired to be monitored. Additionally, or alternatively, encrypted or unencrypted data can be transmitted through an encrypted transmission protocol, such as the wireless encryption protocols (WEP, WPA and WPA2) associated with the IEEE 802.11 wireless protocols or a Bluetooth encryption protocol associated with IEEE 802.15. Any number of other encryption methods can be used to encrypt data in conjunction with the present invention.

In some embodiments, such as described for the system 300, a group of coordinators 120 may be configured to relay communications amongst themselves when fewer than all coordinators 120 are within communication range of a gateway 130.

Data Processing

A calculation of the RPM of a machine may be based on vibration/accelerometer readings.

A baseline “standard operating range” may be determined for individual assets (which are more accurate than manufacturer's generic operating tolerances) and detect events outside the SOP for the particular asset.

Data may be collected for multiple assets over periods of time and generate metrics (expected servicing needed, expected lifespan of parts, effects of heat/cold/other environmental factors on performance), for each asset monitored.

Commands from the Server

In addition to receiving and processing data from the sensor devices 110 and other components operating in conjunction with embodiments of the invention, the server 150 (or user computing device 160 if desired) can transmit a command to control various functions of such components, the asset being monitored, or other systems and devices. Any number of commands of any type may be transmitted by the server 150 to any suitable recipient. The command can be transmitted using the same variety of wired and wireless methods discussed previously. For example, the server 150 may issue a command to control, reconfigure, and/or update a software application operating on the gateway 130, coordinator 120, and/or sensor device 110.

The commands need not be sent directly to a device they are intended to control. For example, a command could be transmitted to a coordinator 120, which in turn retransmits it (unmodified) to the appropriate sensor device 110. Alternatively, the coordinator 120 could receive a command from the server 150, analyze the command, and then transmit an appropriately formatted command tailored to the specific sensor device 110 to be controlled. In this manner, the server 150 need not be able to generate a command for each and every specific device it wishes to control, rather, it can send a command appropriate to a class of sensor devices (e.g., those with vibration sensors) and the coordinator 120 can appropriately translate the command to control the sensor device 110. The commands from the server 150 can initiate/run diagnostic programs, download data, request encryption keys, download encryption keys, and perform any other suitable function on devices operating in conjunction with systems and methods of the present invention.

In any system where commands can be sent remotely, security is always a concern, especially when a wireless implementation may provide an entry vector for an interloper to gain access to components, observe confidential data, and control assets such as expensive oil and gas engines/pumps. Embodiments of the present invention provide for enhanced security in a remote command system while still allowing flexibility and minimal obtrusiveness.

In one embodiment, a command received by any of the components may be authenticated before the command is either acted upon by the destination component, or forwarded to another component in the system. Authentication may be directed to determining (1) whether the command came from a trusted or authorized source, and/or (2) that the recipient is actually the intended recipient of the command. In one implementation, source command authentication is achieved by determining whether the origin of the command is a trusted component or server, and one way to accomplish this determination is analyzing whether a command is properly digitally signed by the originator or some other authentication information is provided that assures the recipient component that the message or command is authentic and the recipient component is actually the intended recipient. In an alternate implementation, destination command authentication is accommodated by examining the contents of the message or an authorization code to determine the intended recipient, or alternatively decrypting the command or a portion of the command to verify the intended recipient.

When commands are created by a command originator, the originator may allow a recipient to verify the authenticity and/or validity of the command by at least one of the following methods: (1) encrypting the command with a private key of the command originator; (2) generating a digest of the command (through a method such as a hashing algorithm discussed above) and optionally encrypting the hashed digest with the command originator's private key, or (3) utilizing a symmetric encryption scheme providing an authentication code (such as a cryptographically hashed password) that is compared to previously stored values. When a system component receives the command along with any encrypted or cleartext certification data, the component may determine the command is valid by: (1) attempting to decrypt an encrypted command message with the alleged originator's public key, (2) attempting to decrypt an encrypted digest with the alleged originator's public key, and comparing the result to a hashed value of the command, or (3) comparing a cryptographically hashed password for the alleged originator to known pre-stored values, and if a match is found, authorization is granted. As an additional step, if the command were optionally encrypted using the intended provider's public key, then only the recipient is capable of decrypting the command, ensuring that only the truly intended recipient devices were being issued commands, and not an unintended third party. For example, authenticating the command may comprise decrypting at least part of the command using at least one of: a public key associated with the server 150; a private key associated with a sensor device 110; and a private key associated with the sensor device 110.

Systems and devices operating in accordance with aspects of the present invention may implement one or more security measures to protect data, restrict access, or provide any other desired security feature. For example, any device operating in conjunction with the present invention may encrypt transmitted data and/or protect data stored within the device itself. Such security measures may be implemented using hardware, software, or a combination thereof. Any method of data encryption or protection may be utilized in conjunction with the present invention, such as public/private keyed encryption systems, data scrambling methods, hardware and software firewalls, tamper-resistant or tamper-responsive memory storage devices or any other method or technique for protecting data. Similarly, passwords, biometrics, access cards or other hardware, or any other system, device, and/or method may be employed to restrict access to any device operating in conjunction with the present invention.

Exemplary Sensor Device

A method according to the invention may be implemented using any suitable system, sensor device (or simply, “device”) or a plurality of devices. A device according to the invention may be mounted on a machine whose parameters it will monitor, or may be remote to the machine. Furthermore, a device may monitor a single machine parameter, such as temperature, or multiple parameters, such as temperature, pressure, vibration and exhaust gas constituents. A device may also monitor one area of a machine, such as one cylinder and/or corresponding valve set, or the exhaust, or it may monitor several areas of a machine. The monitoring may be continuous or periodic, and if monitoring multiple parameters or areas, a device may monitor all simultaneously, or monitor one or more at one time and others at a different time.

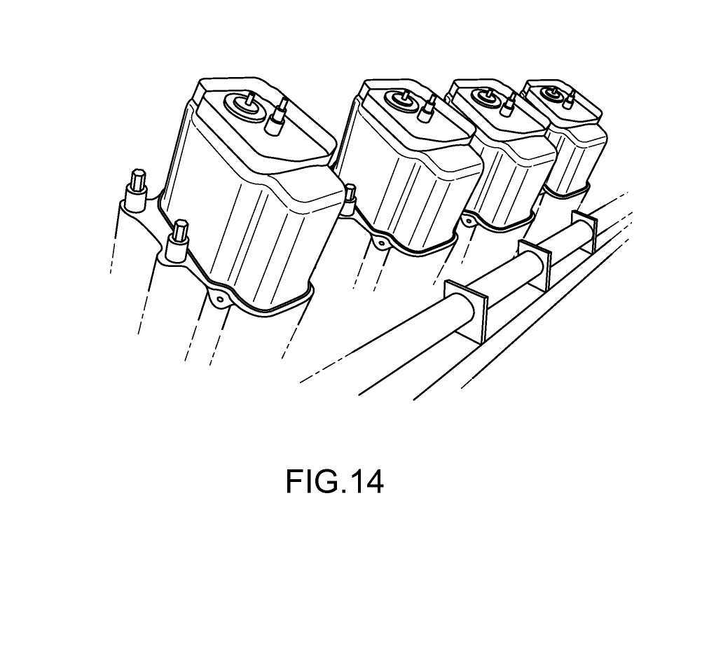

Turning now to FIGS. 14-17, an exemplary device according to the invention are shown as are one or more of the environments in which such a device operates. In this embodiment, the device 110 is mounted on the valve cover of an engine and is appropriately sized for the particular engine on which it is to be mounted, although it may be mounted at any suitable location. For example, it may be mounted remotely to the engine or mounted on or near any device or material which it is to monitor. Further, the device may be of any suitable size required, and its size may vary according to whether it self generates power and the power required for it to operate.

Device 110 as shown measures the temperature and vibration of a single cylinder and valve set for the engine. Thus, in this embodiment, there is preferably a single device 110 mounted on the valve cover associated with each cylinder of the engine, and in one embodiment the engine has sixteen cylinders and utilizes one device 110 for each cylinder.

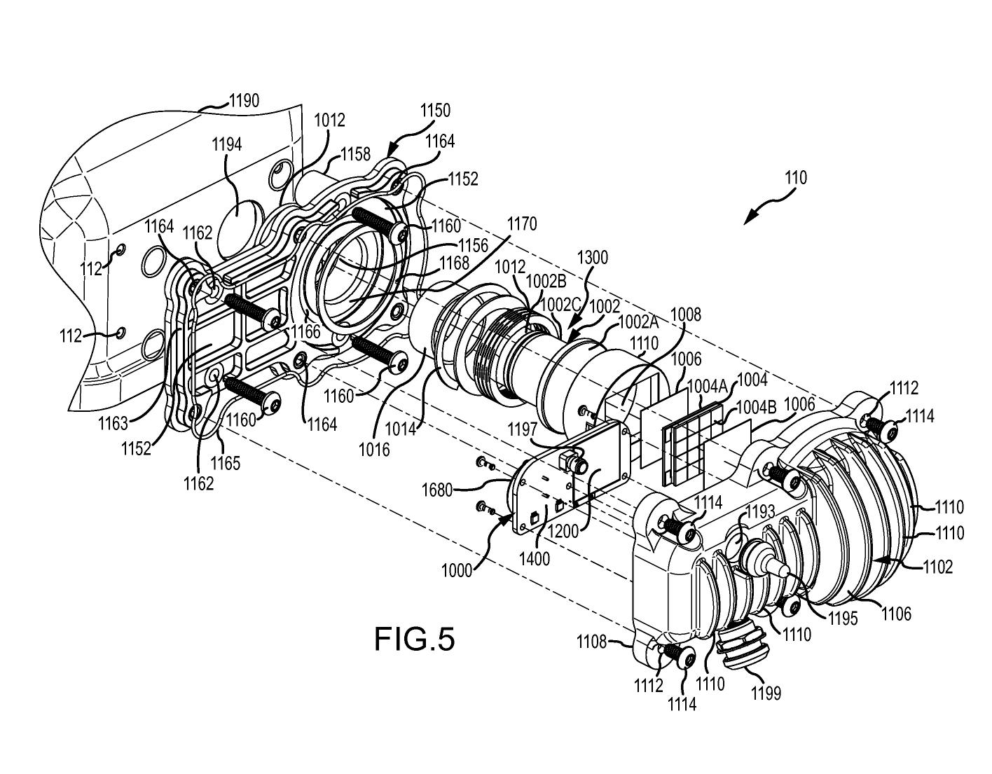

Device 110 is self-contained and is mounted to a valve cover by boring holes 112 into the valve cover to mount the device, and to form an opening for a heat pipe, as described below. Device 110 as shown includes a casing 1100, a printed circuit board 1000, a primary power source 1200 (shown, for example, in FIG. 5), which is preferably a secondary battery, a power generating system 1300 (shown, for example, in FIG. 5, which is a thermoelectric generator, which is also called a thermal energy generator (or “TEG”), a processor 1400 (shown in FIG. 5), and a secondary power source 1680, which is preferably a primary battery. In a preferred embodiment, the TEG powers the primary power source which in turn powers the device. The device could be directly powered by the TEG as well. The purpose of the primary power source is to provide continuous power in case the TEG fails or does not generate sufficient power. The purpose of the secondary power source is to provide backup power if the TEG and/or primary power source fail. An advantage of the TEG is that, by either powering the device or recharging the primary power source, it can reduce or eliminate the need to replace batteries. This is especially important in remote areas where travel costs make replacement expensive, or in areas where there are flammable or explosive gases or liquids present (such as in a natural gas field) and a spark from changing a battery could cause a fire or explosion.

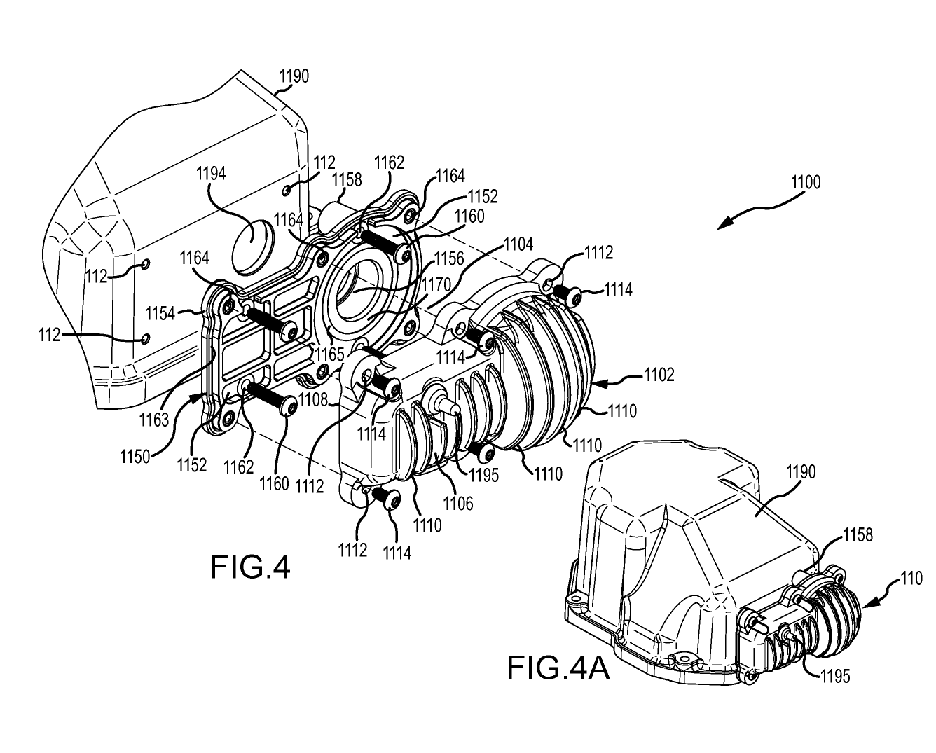

FIG. 4 is an exploded view of a casing 1100.

Casing 1100 has a first part 1102 and a second part 1150. As shown, first part 1102 is farther from the engine than second part 1150, whereas second part 1150 is directly or indirectly mounted to the engine, and in the embodiment shown is mounted to a valve cover 1190. First part 1102 is preferably comprised of a heat conducting material, such as cast aluminum, while second part 1150 is preferably comprised of an insulating material such as plastic. When first part 1102 and second part 1150 are connected they define a cavity 1104 therebetween that houses components of device 110.

The purpose of casing 1100 is to protect the components inside the casing, and any suitable structure for the particular operating environment will suffice. In this embodiment, wherein casing 1110 is mounted on the valve cover 1190 of an engine, the heat of the engine could potentially damage the components inside the casing 1100. It is preferred that the temperature inside cavity 1104 does not exceed 85° C. because that may damage certain components. And, although components could be purchased that can withstand higher temperatures (for example, up to 125° C.), these are currently much more expensive. Therefore, second part 1150 is preferably comprised of insulating material to help prevent heat from the engine from being transferred to cavity 1104, and first part 1102 is preferably comprised of a conductive material to dissipate heat from cavity 1104.

First part 1102 has a top section 1106 and a bottom outer perimeter 1108. Top section 1106 preferably has a plurality of heat dissipating structures 1110. Structures 1110 can be designed in any fashion to dissipate heat without interfering with the function of the device 1000. As shown, structures 1110 are fins extending outward from top section 1106. Structures 1110 may alternatively be, as examples, a plurality of rods or a plurality of rods and fins, but any structure that can dissipate heat may be used.

In this embodiment it is preferred that the fins are spaced between ⅛″ and ⅜″ apart and extend between ¼″ and ⅝″ beyond the surface of top section 1106 at their highest point. The fins are preferably taller at the position of the casing 1100 where the TEG is located in order to dissipate the greater heat associated with the TEG.

Bottom outer perimeter 1108 includes fastener retainers 1112 that retain fasteners 1114 in order to attach first part 1102 to second part 1150. In this embodiment there are six fastener retainers 1112 that accept and retain six fasteners 1114, which in this case are 10-24 button head cap screws, although any suitable fastener may be used.

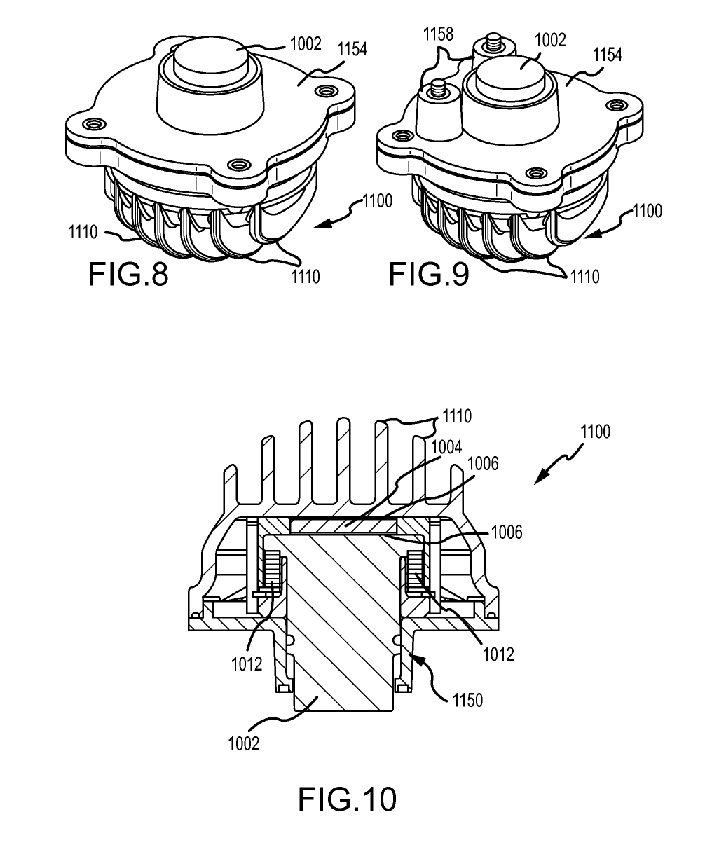

Second part 1150 has an inner surface 1152, an outer surface 1154 (best seen in FIG. 8) and an opening 1156. Outer surface 1154 is generally smooth and is the part of casing 1100 and device 1000 that is closest to the engine (in this embodiment), unless device 110 includes mounting legs, as discussed below. Depending upon the material used and its thickness, outer surface 1154 could be attached directly to a surface, such as a valve cover of the engine, so that it touches the surface (see, for example, FIG. 8). Alternatively, and as shown in the preferred embodiment, outer surface 1154 includes a plurality of mounting legs 1158. Mounting legs 1158 are preferably between ⅜″ and 1½″ long and mount directly to a valve cover 1190 of the engine, or any other suitable surface.

The purpose of mounting legs 1158 is to space device 110 from a hot surface or the otherwise undesirable surface for device 110, such as the hot valve cover 1190 in order to help prevent device 110 from being damaged, such as by becoming overheated. There are preferably two or four mounting legs 1158, although any suitable number can be used.

Preferably, each mounting leg 1158 is attached to a valve cover or other surface by a fastener 1160, which is preferably a 10-24 button head cap screw. Any suitable fastener may be used and in this embodiment each mounting leg 1158 has an opening 1162 extending therethrough and a metal screw boss in each opening 1162. Each screw boss receives a fastener 1160. Fastener 1160 is threadingly received in each screw boss and threadingly received in fastener openings 1160 and, as shown, openings 112 on valve cover 1190.

Inner surface 1152 has a channel 1163 for retaining a gasket 1165. When first part 1102 is attached to second part 1150 a lip on the bottom outer perimeter 1108 (not shown) is received in channel 1163 and compresses gasket 1165 to form a seal to help keep dust and moisture out of cavity 1104.

Inner surface 1152 includes fastener retainers 1164, which are openings that receive metal screw bosses. Fastener retainers 1164 receive fasteners 1114 in order to attach first part 1102 to second part 1150.

Opening 1156 is configured to permit a heat pipe (described below) to pass therethrough. Opening 1156 is of any suitable size. Surrounding the opening 1156 is a second channel 1166 for retaining a gasket 1168, wherein gasket 1168 creates a seal against the heat pipe to seal cavity 1114 from the outside environment. Also surrounding opening 1156 is a depression 1170 that creates a space for retaining an insulating sleeve (described below) that surrounds the heat pipe and helps to keep its heat from dissipating into cavity 1114.

A valve cover 1190 is also shown in FIG. 4. Valve cover 1190 has been modified from its original configuration by adding fastener openings 192 and a heat pipe opening 1194.

First part 1102 also includes an opening 1193 through which an antenna (not shown), which attaches to connector 1197, which is in turn connected to PCB 1000, so as to send and receive signals wirelessly to and from PCB 1000, can extend and a protective sheath 1195 that covers and protects the antenna. It is preferred that the cover for the antenna be made of a material that is resistant to the environmental in which device 110 is placed and that the antenna extends far enough so that it is higher than any of the heat-dissipating fins or rods so that signals emanating from or received by the antenna are not partially blocked by these structures.

There may be more than one PCB 1000 (or PCBA, meaning printed circuit board assembly), and in a preferred embodiment, the one or more PCBs include: (a) the primary power source, which is preferably a secondary battery, (b) the secondary power source, which is preferably a primary battery, (c) a radio, such as a Bluetooth 4.0 module, (d) a microcontroller, (e) a clock, (f) an energy harvesting managing circuit, (g) one or more capacitors, (h) an accelerometer, (i) an antenna connection, (j) a thermocouple amplifier, (k) a resistor SMD, and (l) an inductor. The PCB may be two sided.

There are also one or more additional openings (not shown) that may receive or include a plug 1199 or other wired connection for receiving operational data about one or more operating parameters of the engine, as described above. Plug 1199 may connect to a thermocouple through a wired connection to receive temperature data or connect to a device to receive vibrational data or any other type of data. Alternatively, the device 110 may receive operating data wirelessly.

FIG. 5 shows an exploded view of device 110 according to an aspect of the invention. A TEG assembly 1300 includes a heat pipe 1002, which is preferably comprised of a thermally conductive material such as ceramic alumina or any other suitable material. The purpose of heat pipe 1002 is to transfer heat from a heat source, which in this case is an engine, to a device that utilizes the heat energy to generate electricity (such as TEG 1004) to either recharge the primary power source of device 110 or to directly power device 110. Any energy source, such as solar energy, or a piezo device that generates energy when vibrated, can also be used to recharge the primary power source or power device 110, but in any event it is preferred that the energy source be present in and collect energy from the ambient environment (either as part of or near device 110) and not be a separate energy source, such as electricity from an outlet. This is because device 110 is preferably self-contained and capable of operating without requiring hardwiring to an energy source. Further, hard-wired power may not be available where device 110 operates and/or may be dangerous if device 110 is in a flammable or potentially explosive environment, such as a natural gas field. If TEG assembly 1300 or another ambient energy source is used to directly power device 110, it is possible that no battery power be used. Further, even if a primary power source, such as a battery, is used, the secondary, or back up power source, is optional.

Heat pipe 1002 has a first end 1002A, a second end 1002B, and a body portion 1002C. First end 1002A is in thermal communication with TEG 1004. TEG 1004 receives heat from first end 1002A and converts it into electricity, and has wires that transmit the generated electricity. The wires may be connected to a PCB 1000, or directly to the first power source, or to any suitable location to operate device 110. In this embodiment, for thermal energy generator 1004 to generate sufficient electricity, first end 1002A should be at least 10° C. hotter than the ambient temperature inside of cavity 1104.

To increase the heat transfer between the first end 1002A and TEG 1004, a conductive sheath 1006 is placed between the two. The sheath is primarily comprised of graphite or another conductive, soft material. Sheath 1006 is preferably 1/32″ or less in thickness and it conforms to the surface of first end 1002 and to the surface of TEG 1004, thereby effectively increasing the surface area available for transferring heat.

TEG 1004 has a first side 1004A that is adjacent first end 1002A of heat pipe 1002 and a second side 1004B adjacent an inner wall of first part 1102 of casing 1100. Heat not converted into electricity by TEG 1004 is conducted through second side 1004B to first part 1102 of casing 1100, where it is conducted out of device 110. This helps to prevent cavity 1114 of device 110 from overheating.

A second sheath 1006 is preferably positioned between second side 1004B of thermal energy generator 1004 and the inner wall of first part 1102, again in order to increase the surface area and heat transfer between the two in the manner described above.

In this embodiment, the first end 1002A of heat pipe 1002 has a larger diameter than the rest of heat pipe 1002 and includes an opening 1008. Opening 1008 is for retaining TEG 1004 and the sheath 1006 that is between heat pipe 1002 and TEG 1004. First end 1002A is preferably covered at least partially by an insulating material, which is preferably plastic sleeve 1010, to help keep heat from dissipating into cavity 1114.

An o-ring 1012 is used as a secondary seal on heat pipe 1002 to help seal cavity 1104 from the outside environment.

Heat pipe 1002 is biased towards thermal energy generator 1004 by a spring 1012 positioned around body portion 1002C. The purpose of the biasing is to press end 1002A against thermal energy generator 1004 and/or, or against sheath 1006, to enhance the heat transfer to thermal energy generator 1004. If heat pipe 1002 is biased, any suitable structure or method may be used to generate a pressure fit between the heat pipe and (directly or indirectly) the TEG 1004. In one embodiment the biasing force is about 100-200 psi, or about 170-250 psi, or about 200 psi.

Heat pipe 1002 is also held in position in cavity 1104 of casing 1100 by a locking ring 1014 positioned around body portion 1002C and under spring 1012. Locking ring 1014 fits into depression 1170 and holds heat pipe 1002 in position. The body portion 1002C adjacent opening 1156 is at least partially surrounded by an insulating material, and in this embodiment is surrounded by plastic sleeve 1016, which helps prevent heat from dissipating into cavity 1104.

Second end 1002B of heat pipe 1002 extends through opening 1156 in order to receive heat from a heat source. In this embodiment, the heat source is the engine. Second end 1002B preferably extends out of casing 1100, through opening 1194 in valve cover 1190 and is retained inside of the valve cover. The heat pipe 1002 receives sufficient heat to generate electricity through TEG 1004. Furthermore, by not contacting the engine or valve cover 1190 directly, little or no vibration is transferred through the heat pipe 1002 to device 110.

Processor 1020 is preferably a PCB chip 1000 with circuitry that preferably performs the following functions (some of which were noted above). First, it converts power from the TEG assembly 1300 into electricity suitable for charging the power source of device 110, or for operating device 110 directly. Second, it includes an accelerometer capable of measuring vibration. Third, it may also be capable of receiving and analyzing (in whole or in part) operational data other than vibrational data, such as temperature, chemical analysis of materials such as a liquid, solid or gas, pressure, or exhaust gas data, and potentially convert any data it measures or receives into digital form so that it can be stored, analyzed and/or transmitted.

Processor 1020 is in direct or indirect communication with the power source, the thermal energy generator, one or more data inputs, and a transmitter to transmit data.

A primary power source 1022 is preferably a solid state, thin film LiPON battery attached to processor 1020. A secondary power source 1024 is preferably a lithium thynol chloride wafer cell and operates only if power source 1022 fails.

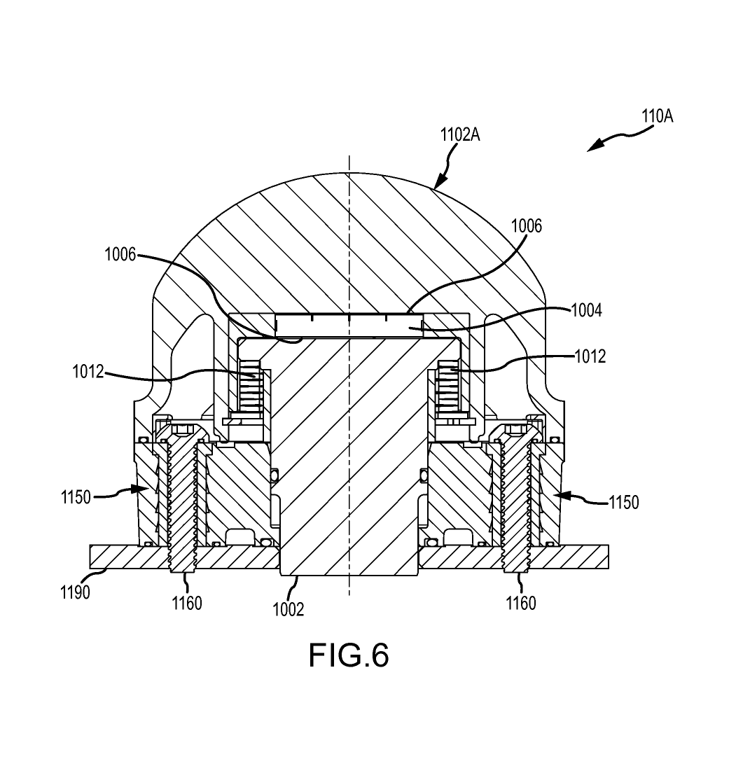

FIG. 6 is a cross-sectional view of an alternate device 1100A according to the invention that is the same in all respects as the previously described device 110 except that it has no heat dissipating structure on top portion 1102A of the casing.

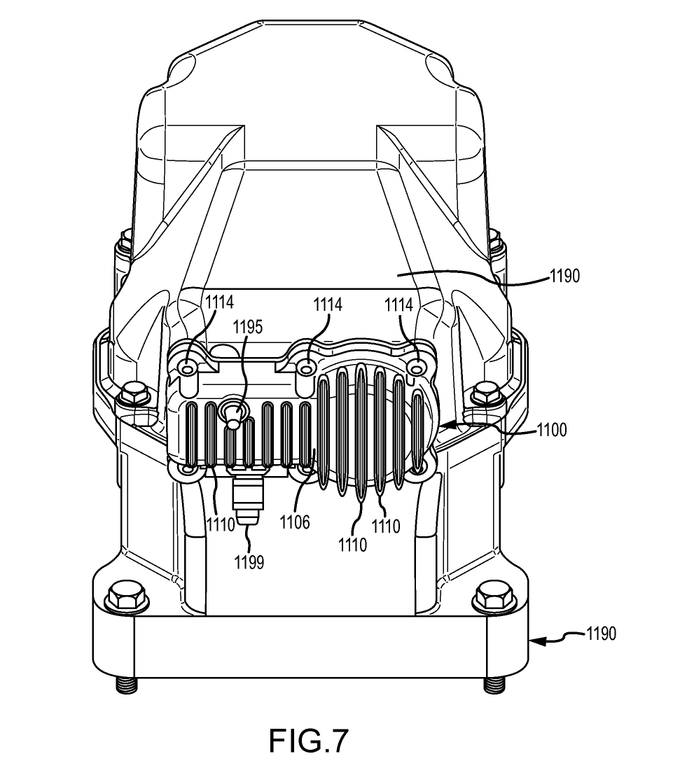

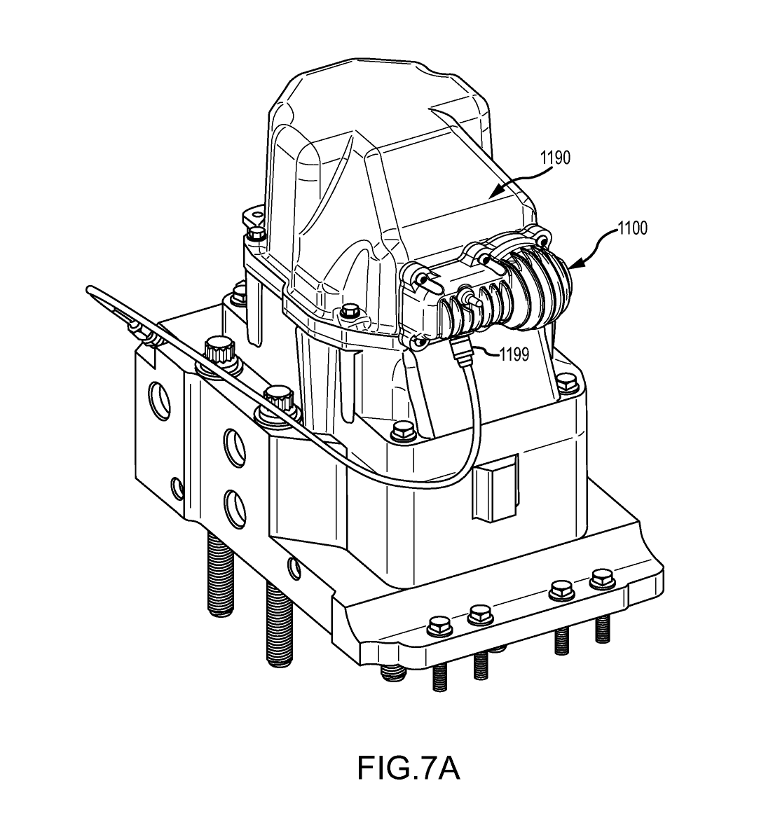

FIG. 7 is a perspective, front view of the assembled device of FIGS. 1-5 mounted on the valve cover of an engine. FIG. 7A is a different perspective view of the arrangement in FIG. 7 showing a wired connection between device 110 and a thermocouple positioned in the engine.

FIG. 8 depicts an embodiment of the invention with a flat outer surface 1154 for mounting directly to another surface, such as the surface of a valve cover.

FIG. 9 depicts an embodiment of the invention with two mounting legs 1154 (although any suitable number may be used) to mount to a surface and create a space between surface 1154 and the surface to which the device is mounted.

FIG. 10 is a cross-sectional view of the device of FIG. 8.

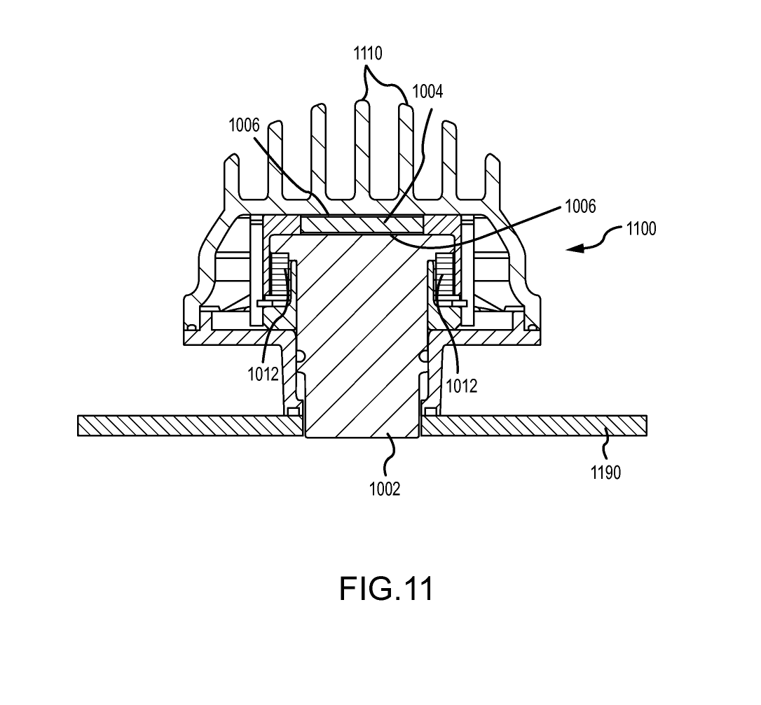

FIG. 11 is a cross-sectional view of the device of FIG. 8 mounted on a valve cover wherein the heat pipe 1002 protrudes through the opening 1194 of the valve cover.

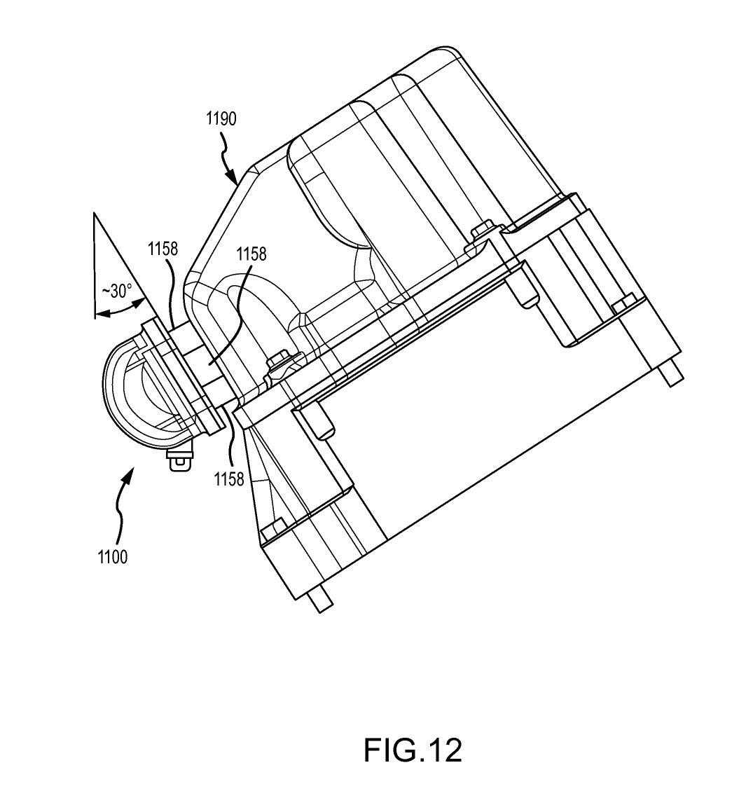

FIG. 12 shows a device according to the invention that has a plurality of legs 1158 (preferably four) for mounting the device to a valve cover 1190.

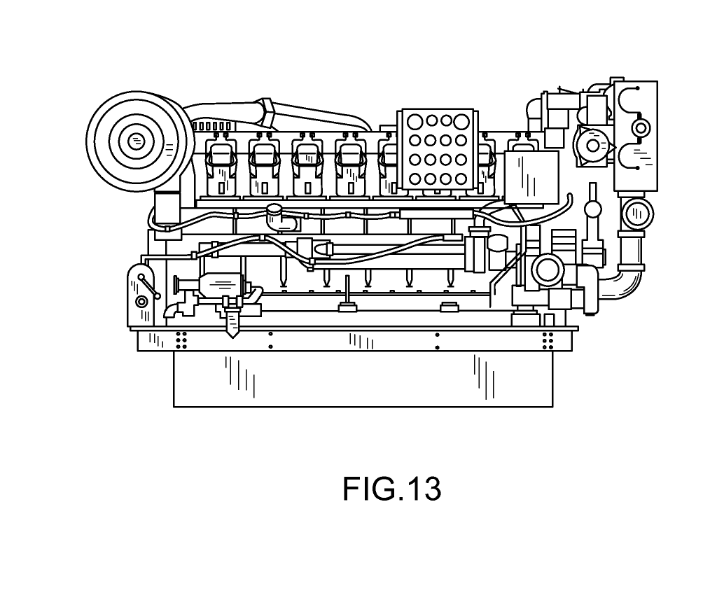

FIG. 13 shows one type of machine, which is a 16-cylinder diesel or natural gas engine, on which a device according to the invention may be used.

FIG. 14 is a close-up view of the cylinder heads of the engine of FIG. 13, which is used for natural gas compression. A device according to the invention could be mounted on one or more of the cylinder heads or mounted elsewhere and, in either event, monitor operating parameters of the cylinder and/or the valves associated therewith.

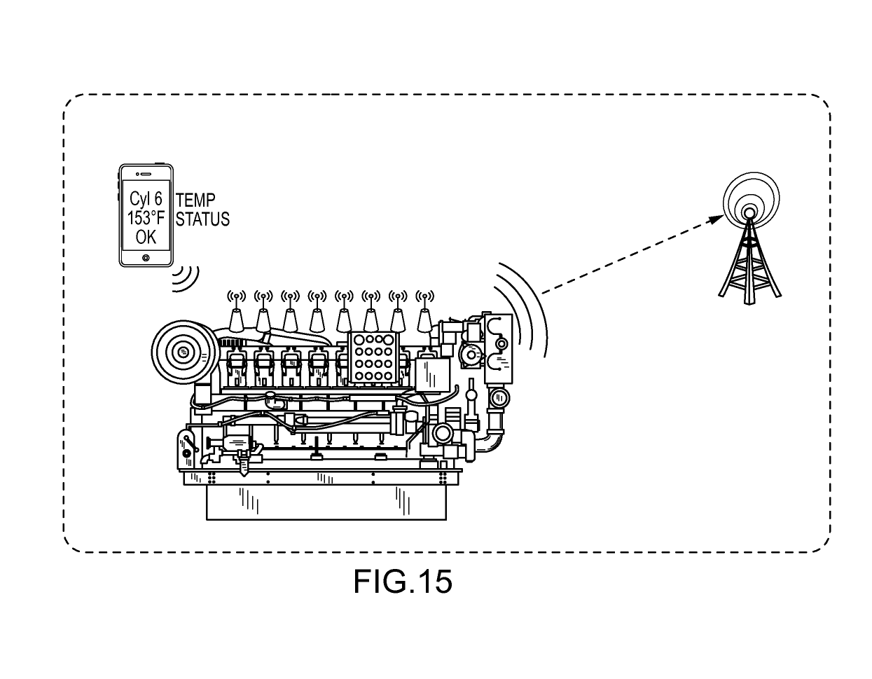

FIG. 15 depicts a device according to the invention being connected to, or otherwise in communication with, each valve of the engine depicted in FIGS. 13 and 14, wherein the device measures parameters associated with each cylinder and/or valve set, or other parameters, and relays the measurements to a gateway. The device and/or the gateway may filter, sort, store and/or analyze all or part of the data either continuously or in for time interval. The device may also be used to harvest information from another machine, such as a compressor.

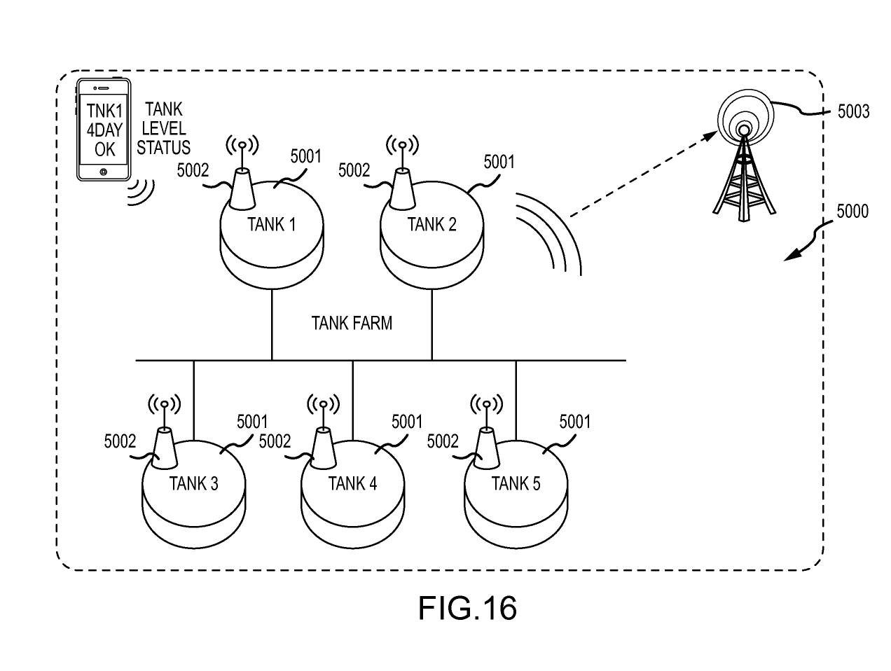

FIG. 16 depicts a tank farm 5000, wherein each farm has a configuration generally as shown in FIG. 1 or in any suitable configuration. The assets being monitored in each tank farm could be one or more of any type of machine, device or material, such as one or more engines, compressors, storage tanks or pipes through which compressed gas passes. A device according to the invention could monitor any desired parameter of any piece of equipment and send the information to a coordinator 5002 that in turn could relay it to a gateway 5003 that could send all or part of the information via any suitable transmission medium to another location. Using this system, information may be sent in any suitable manner, such as raw or compressed data sent continuously, intermittently according to a schedule that may be altered, or when the system 5000 senses that there is a problem and/or is aware that the data transmission costs are low. The data can be gathered, stored, analyzed, combined and compared to other data in any suitable manner by system 5000 prior to or after transmission. System 5000 may also receive signals to reconfigure any of the operating logic of any device in system 5000.

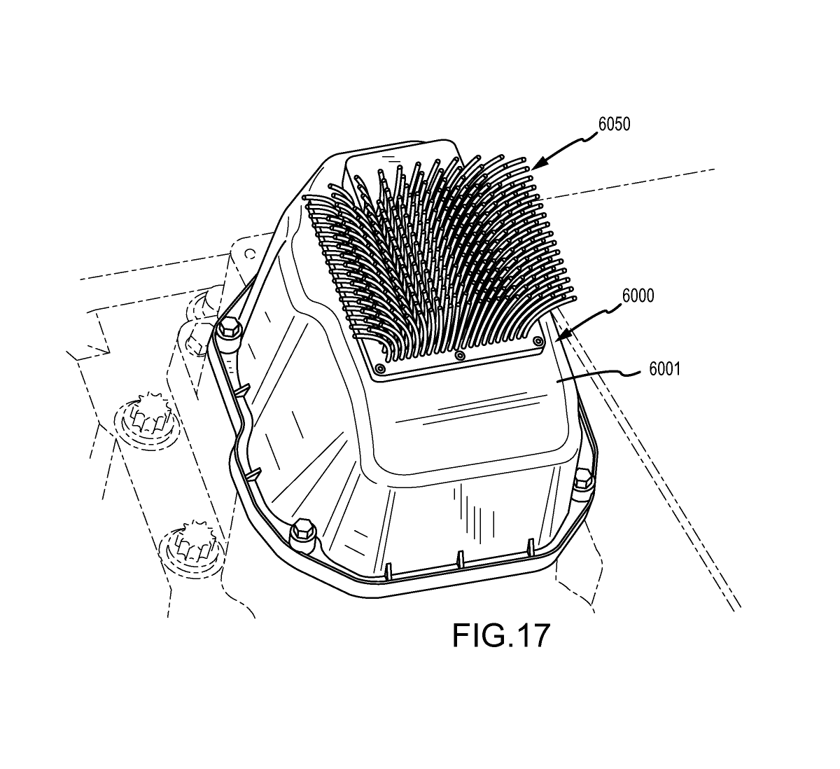

FIG. 17 depicts a valve cover 6000 specifically designed to include a version of device 110, which is preferably large in size. In all respects, the previously described devices are the same as the device in valve cover 6000 except that the top surface 6001 of valve cover 6000 may also form the top surface of the device and is for dissipating heat. As shown, valve cover 6000 includes a heat dissipation structure 6050 that comprises a plurality of rods, although the previously described fins may also be utilized, or a combination of fins and rods may be utilized. Cover 6000 is preferably comprised of steel.

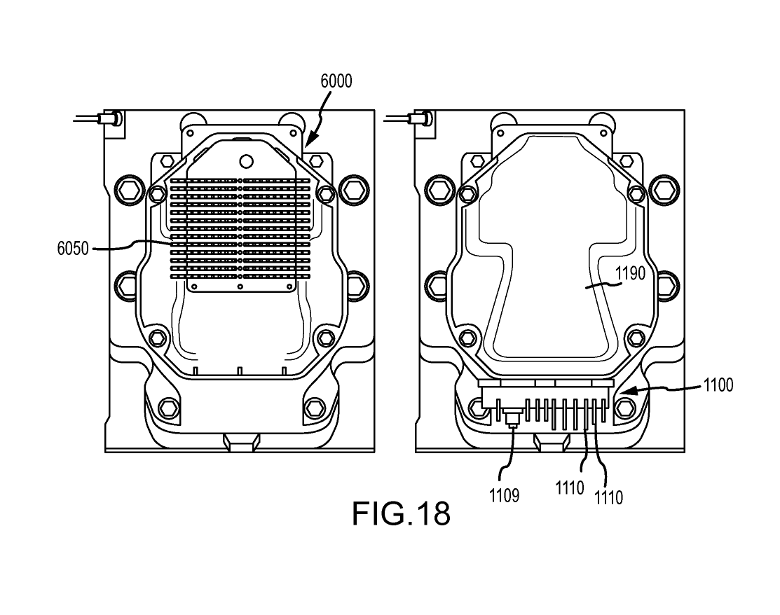

FIG. 18 shows a comparison of device 110 to cover 6000. Device 110 is preferably sized to mount to a single cylinder or valve cover 1190 (as shown) and because of its size (practically) generates only enough power to monitor the parameters associated with a single cylinder/valve combination, unless additional power is provided from a modification of device 110 or from another source.

Valve cover 6000 can house a larger version of a device according to the invention and can power many other monitoring devices, or other equipment, through the accessible ambient heat energy. The electricity generated would be transmitted from valve cover 6000 to other devices or equipment through wires. Further, valve cover 6000 could also include its own internal and/or external structures as previously described for device 110.

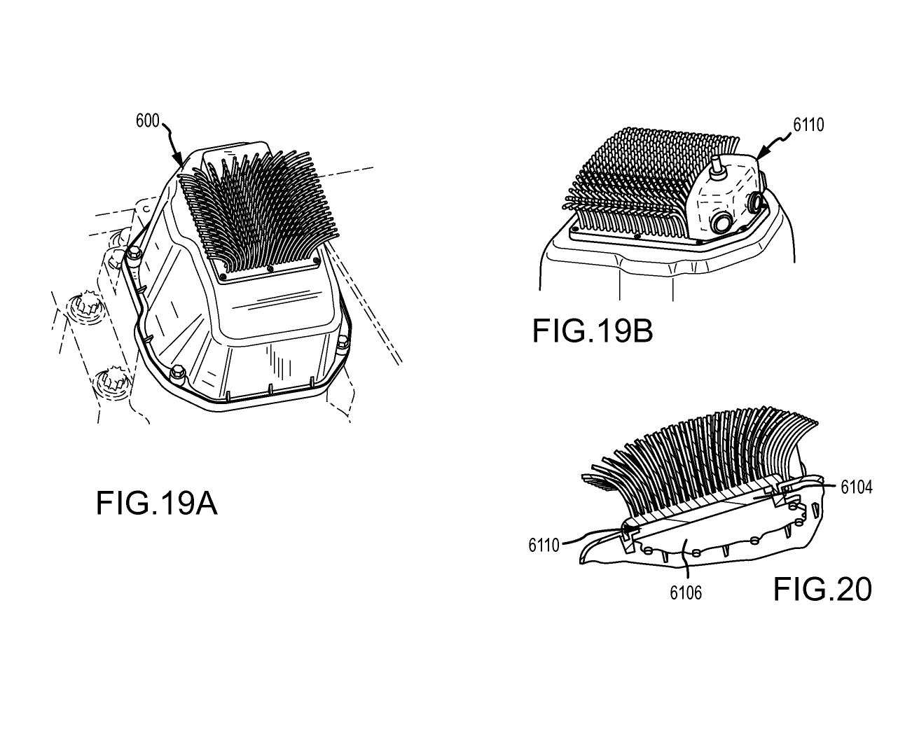

FIGS. 19A, 19B and 20 show various views of the valve cover 6000 and show how a device 6110 could be positioned on top of or partially inside of valve cover 6000. In this case, there is no need for a heat pipe because the TEG 6104 has a plate beneath it that transfers heat from the cavity inside of valve cover 6000 to TEG 6104 to generate electricity.

Another embodiment of an aspect of the invention is a drilling pipe with a vibrational measuring and recording device. The pipe is preferably of a type used for drilling oil or natural gas wells and is known in the art. The pipe is comprised of sections, usually 42 feet in length, that are threaded together. Over time the pipe wears and can break, either at the threaded portion or elsewhere. If the pipe breaks during usage, it could create delay and expense because if, for example, the pipe is several thousand feet underground it may be difficult or impossible to retrieve and another hole must be bored. The wear on a pipe is a function of at least, (1) the number of times the pipe has been used, which can be determined by the total number of turns the pipe has made, and (2) the type of earth in which the pipe has been used, for example, if the pipe is used in soft soil the wear on the pipe is less than if the pipe is used to drill through rock.

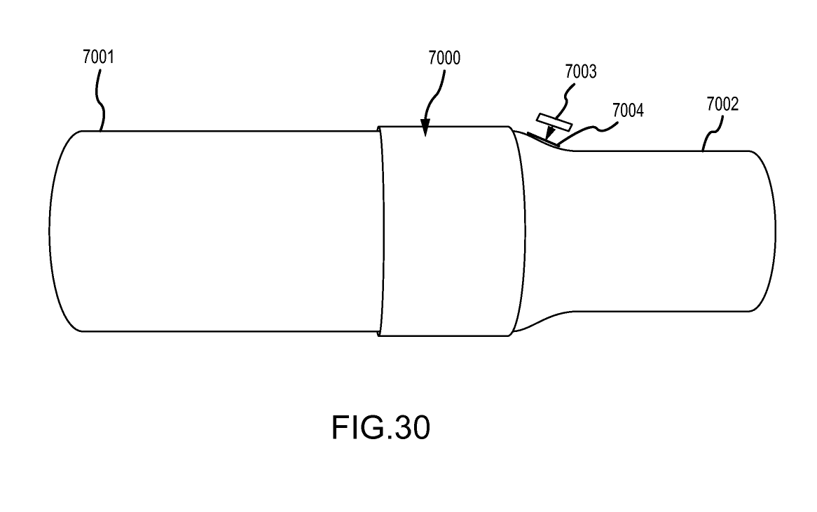

The wear on a pipe can be measured by the vibration to which it has been exposed, which can measure (or approximate) the number of turns and the stress due to the type of earth in which it has been used. Turning now to FIG. 30, a section of a pipe 7000 according to the invention is shown. End 7001 has a larger cross-sectional area than end 7002, which is meant to be threaded into end 7001 of another pipe section. Attached to pipe section 7000 is a power source comprising a piezo chip that generates electricity when subject to vibration, an accelerometer that measures vibration to which pipe section 7000 is subjected, and a memory to store the vibrational data. The power source, accelerometer and memory are preferably all part of one, flat unit 7003, so they extend very little from the surface of pipe section 7000. Preferably unit 7003 is contained in a recess 7004 of between ⅛″ and 5/16″ deep formed in pipe section 7000, and most preferably the recess is at end 7002, which has less direct contact with the earth as the drilling progresses. The memory of the unit can be read or downloaded in any suitable manner, such as by using an RF reader.

Using this device, users can determine when a pipe section has reached the end of its useful life for their purposes and either discard or sell the pipe section. A predetermined vibrational life span of the pipe has been exposed and can be compared to this known vibrational life.

Communications

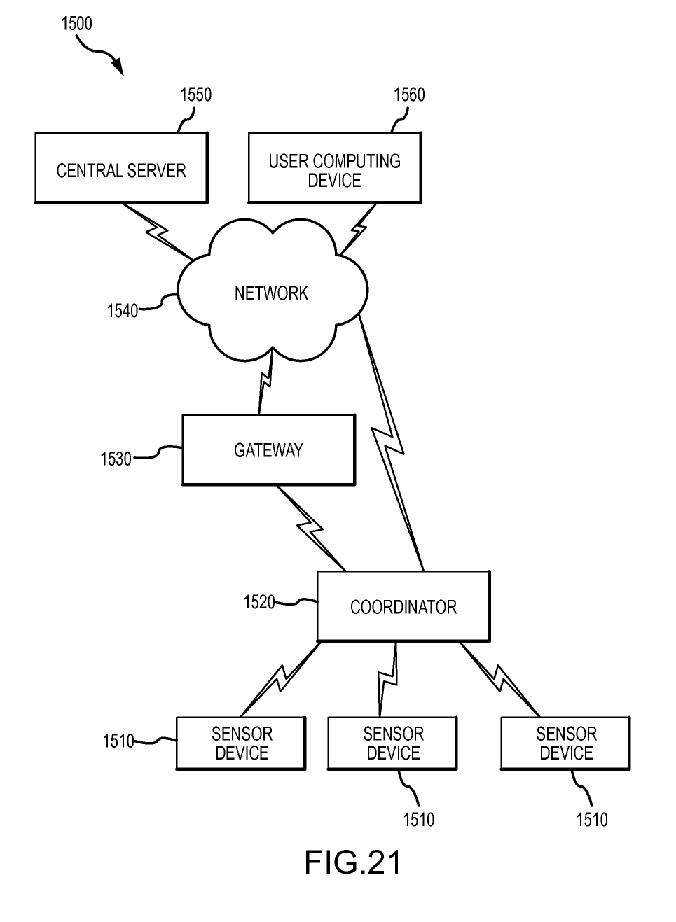

In accordance with various embodiments and with reference to FIG. 21 (and FIG. 1), communication architecture for a remote sensing system 1500 can comprise at least one sensor device 1510 communicating with a coordinator 1520. The coordinator 1520 communicates with central server 1550 and user computing device 1560 via gateway 1530 and/or network 1540. Sensor devices 1510 may be referred to herein as “motes,” and coordinators 1520 may be referred to as “nodes.” The functionality of the sensor device 1510, coordinator 1520, server 1550, computing device 1560, gateway 1530 and/or any other component operating in conjunction with the present invention can be implemented in any suitable manner, such as through a processor executing software instructions stored in a memory. Functionality may also be implemented through various hardware components storing machine-readable instructions, such as application-specific integrated circuits (ASICs), field-programmable gate arrays (FPGAs) and/or complex programmable logic devices (CPLDs).

The sensor device 1510 receives data collected from one or more connected sensors, and can be configured to transmit the collected data to the coordinator 1520. Furthermore, in various embodiments, sensor device can be configured to transmit the collected data to coordinator 1520 in real-time or batch format. As used herein, “real-time” is defined to mean intervals measured in minutes. For example, the sensor data may be transmitted every 5 minutes, 10 minutes, 30 minutes, or the like. Furthermore, the coordinator 1520 can be configured to transmit data to the central server 1550 via the gateway 1530 and/or the network 1540. Within the remote sensing system 1500, data can be communicated using a variety of communication methods. For example, data may be communicated via a wireless connection or a wired connection. In various embodiments, a wireless communication device can be configured to transmit using at least one of a satellite communication network, a local area network (LAN), a wide area network (WAN), a wireless mobile telephone network, a General Packet Radio Service (GPRS) network, a wireless local area network (WLAN), a Global System for Mobile Communications (GSM) network, a Personal Communication Service (PCS) network, and an Advanced Mobile Phone System (AMPS) network. Moreover, data can be directly downloaded from the sensor device or aggregating computer using a cable connection to a computing device.

The components of the remote sensing system 1500, namely the sensor device 1510, coordinator 1520, gateway 1530, and central server 1550, may include, or operate in conjunction with, any type and number of transceivers. In various embodiments, the components includes a cellular radio frequency (RF) transceiver and may be configured to communicate using any number and type of cellular protocols, such as General Packet Radio Service (GPRS), Global System for Mobile Communications (GSM), Enhanced Data rates for GSM Evolution (EDGE), Personal Communication Service (PCS), Advanced Mobile Phone System (AMPS), Code Division Multiple Access (CDMA), Wideband CDMA (W-CDMA), Time Division-Synchronous CDMA (TD-SCDMA), Universal Mobile Telecommunications System (UMTS), and/or Time Division Multiple Access (TDMA). The transceiver may communicate using any other wireless protocols, such as a Zigbee protocol, a Wibree protocol, an IEEE 802.11 protocol, an IEEE 802.15 protocol, an IEEE 802.16 protocol, an Ultra-Wideband (UWB) protocol, an Infrared Data Association (IrDA) protocol, a Bluetooth protocol, and combinations thereof.

Furthermore, the components of the remote sensing system 100 can be configured, alternatively (or additionally), to communicate using any other method of wired or wireless communication. For example, in various embodiments the transceiver may be configured to communicate using one or more wired connections using, without limitation: tip and sleeve (TS), tip, ring, and sleeve (TRS), and tip, ring, ring, and sleeve (TRRS) connections; serial peripheral interface bus (SPI) connections; universal serial bus (USB) connections; RS-232 serial connections, Ethernet connections, optical fiber connections, and Firewire connections. The transceiver can be configured (e.g. through a software program residing in memory and executed by processor) to detect and switch to different communication protocols and/or different wired or wireless connections, thus allowing communications with a wide variety of devices.

The coordinator 1520, according to various embodiments, can be a local computer machine located near one or more sensor devices 1510, such that the coordinator 1520 and sensor devices 1510 can communicate using RF signals. Moreover, the coordinator 1520 can be configured to communicate using any desired wired or wireless communication connection or protocol, including those described above. In various embodiments, the coordinator 1520 can be configured to communicate with a plurality of sensor devices 1510 and, in turn, communicate with other coordinators 1520, or the central server 1550. In this manner, a single coordinator 1520 can communicate with multiple sensor devices 1510 using a short-range, low-power communication protocol (e.g., Bluetooth®) and communicate with other systems (such as the central server 1550) using a longer-range protocol, resulting in less overall power consumption by embodiments disclosed herein.

The data communicated in the remote sensing system 1500 may be of two different types, referred to as “smart data” and “dumb data.” The dumb data can be all the data collected by the sensor device 1510. The dumb data can be unfiltered and may be voluminous, as the sensor device 1510 collects a large quantity of sensor data. In contrast, the smart data can be a filtered, summarized, condensed, or reduced subset of the dumb data, or an analysis output. For example, the sensor device may record temperature at a predetermined first time interval. The dumb data would include every temperature recording, whereas the smart data could be the average temperature over a predetermined second time interval, where the second time interval can be greater than the first time interval. Transmitting the average temperature smart data can be more efficient than transmitting the temperature recording dumb data due to the decrease in data transmitted. However, for most purposes there is little to no drop in analysis quality of the data since the smart data provides sufficient information for analysis.

The sensor device 1510 may be configured to detect and transmit data from any number of different sensor units in which it is in communication. Additionally, the sensor device 110 may be configured to perform any desired analysis of the data from the sensor units, including those described below.

In one embodiment, coordinator 1520 has a large amount of memory capable of storing all data transmitted by the one or more sensor devices 1510. For example, the coordinator 1520 may have over a terabyte of storage. In various embodiments, the coordinator 1520 can receive all the “dumb” data from the sensor device 1510. The coordinator 1520 then processes the dumb data into relevant smart data to be transmitted to the central server 1550. Furthermore, the coordinator 1520 can store the dumb data for later retrieval. The dumb data can be manually downloaded later for additional analysis.

The smart data can include an identifier corresponding to the data source, thereby identifying which sensor device 1510 gathered the particular smart data. In various embodiments, the coordinator 1520 can be in communication with multiple sensor devices 1510. Each sensor device 1510 can communicate with the coordinator 1520 using a different frequency. For example, the sensor devices 1510 may transmit within the frequency range of about 868 MHz to about 915 MHz. The coordinator 1520 can use the communication frequency to associate the data with a specific sensor device 1510.

In various embodiments, the coordinator 1520 can communicate to the central server 1550 either via satellite or cellular towers. Furthermore, the coordinator 1520 can be configured to transmit batch data to the central server 1550 at selected times. For example, the batch data transmissions may occur during off-peak times in order to be more cost effective. In other embodiments, the coordinator 1520 can store all the dumb data to be manually downloaded at some point in time.

In accordance with various embodiments, the data processing can be managed in multiple ways. For example, in a first embodiment, the sensor device 1510 can be configured to process, or at least partially process, the data. In a second embodiment, the coordinator 1520 can be configured to process, or at least partially process, the data. In a third embodiment, the central server 1550 can be configured to process, or at least partially process, the data. In a fourth embodiment, the data processing can be managed by any combination of the first, second, or third embodiments of data processing. For example, the sensor device 1510 can be configured to preprocess data for simple tasks, such as determining a change in temperature. The coordinator 1520 can configured to perform more complex data processing, or any processing not handled by the sensor device 1510.

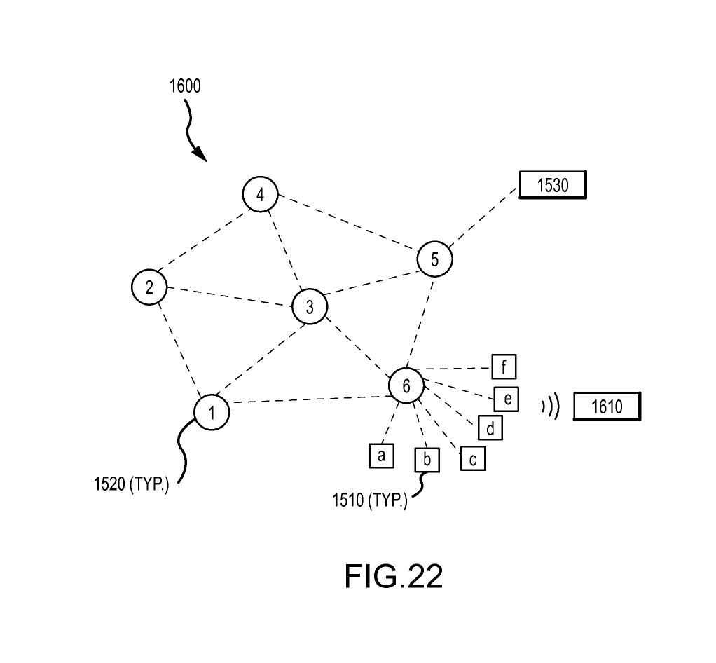

Referring now to FIG. 22, a network of coordinators 1520 (labeled 1-6) is shown, with each coordinator corresponding to a respective engine being monitored. As shown for coordinator #6, each coordinator (1-6) communicates with a respective plurality of sensor devices 1510 (labeled a-f with respect to coordinator #6) via short-range wireless protocol (Bluetooth® in this example). In this exemplary embodiment, coordinators 1-6 communicate with each other and/or with gateway 1530 using a longer-range wireless protocol (an 802.15 protocol in this example) and adjacent coordinators are no more than about 300 feet from each other. At this range, the coordinators can communicate with immediately adjacent coordinators (shown by the dotted lines between coordinators), but only coordinator #5 is within range of gateway 1530. In such cases, coordinators operating in conjunction with embodiments of the invention may be configured to relay communications so that all coordinators can communicate with or through the gateway 1530.

For example, coordinator 4 may transmit data to coordinator 5 for rebroadcast to gateway 1530. Likewise, coordinator 1 may transmit data to gateway 1530 through coordinators 3 and 5. In various embodiments, communications can be alternately relayed through different coordinator nodes to help avoid over-burdening any one particular node. For example, coordinator 1 may first communicate with gateway 1530 via coordinators 6 and 5, and next communicate with gateway via nodes 3 and 5.

As also shown in FIG. 22, sensor devices 1510 (labeled a-f in FIG. 22) can communicate with local device(s) 1610. This allows, among other things, technicians to communicate directly with sensing devices 1510 (to perform diagnostics or other functions) without having to access network 1540 or server 1550.

Gateway 1530

The gateway 1530 communicates with coordinator 1520 and with other systems (such as central server 1550 and user computing device 1560) via network 1540. In various embodiments, such as in the exemplary system 1600 depicted in FIG. 22, gateway 1530 is disposed within communication range of at least one coordinator 1520. In various embodiments, gateway 1530 communicates with one or more coordinators 1520 using a first wireless communication protocol (e.g., an 802.15 protocol) and communicates with network 1540 using a second wired or wireless communication protocol (e.g., a longer-range protocol such as a cellular protocol), including those described previously. Among other things, the gateway 1530 helps maximize the efficiency of the overall power consumption of system 1600 and other embodiments of the invention by using short range (and lower power) communication protocols between sensor devices 1510 and coordinators 1520 and a longer range protocol to communicate with remote devices via network 1540.

In the exemplary embodiments depicted in FIGS. 21 and 22, gateway 1530 includes multiple transceivers to communicate (simultaneously if desired) using different communication protocols, thus allowing the gateway to, for example, communicate with a coordinator 1520 and central server 1550 via network 1540 at the same time. The gateway 1530 may also be configured to store and process information collected from the sensors 1510. The gateway 1530 can thereby provide a technician with local access to data accessible via a mobile computing device 1610 and retain a copy of data in case of a hardware or communication failure related to server 1550.

While coordinator 1520, gateway 1530, and network 1540 are shown as separate components in FIG. 21, alternate embodiments may perform the functionality of these components using a single system or device. Additionally, various embodiments may use more or fewer components to collect data from the sensor devices 1510.

Network 1540

The network 1540 allows the sensor devices 1510, coordinator 1520 and/or gateway 1530 to communicate with other systems and devices, such as central server 1550 and user computing device 1560. The network 1540 may include any combination of wired and wireless connections and protocols, such as those described above. The network 1540 may comprise a local area network (LAN), wide area network (WAN), wireless mobile telephony network, General Packet Radio Service (GPRS) network, wireless Local Area Network (WLAN), Global System for Mobile Communications (GSM) network, Personal Communication Service (PCS) network, Advanced Mobile Phone System (AMPS) network, and/or a satellite communication network. In various embodiments, network 1540 includes the Internet to allow the central server 1550 or computing device 1560 to communicate with sensor devices 1510, coordinator 1520 and/or gateway 1530 from anywhere an internet connection can be established. As such, embodiments of the invention provide efficient, centralized monitoring of assets even in applications (such as oil and gas production) where monitored assets are in remote locations and often spread across large areas.

Central Server 1550

In the exemplary embodiment depicted in FIG. 21, the central server 1550 receives and analyzes data from the sensor devices 1510 and can issue commands to control sensor device 1510, coordinator 1520, gateway 1530, and/or an asset being monitored. The central server 1550 may receive data from the sensor devices 1510 in any desired manner. In various embodiments, the server 1550 can be configured to automatically request data from one or more of the sensor devices 1510 via the network 1540, gateway 1530, and coordinator 1520. Alternatively, the sensor device 1510, coordinator 1520, gateway 1530, or any other device operating in conjunction with embodiments of the invention can be configured to automatically request and/or transmit data in any suitable manner. For example, each sensor device 1510 may be configured to collect and send data regarding vibrations measured from a monitored asset (such as an internal combustion engine or compressor) and automatically transmit such data to the coordinator 1520 at periodic intervals (e.g., every 15 seconds). The coordinator 1520, in turn, may immediately retransmit the data to the server 1550 via network 1540 and/or to gateway 1530, or may store the data for analysis and/or later transmittal.

The transmission of data by a device operating in conjunction with the present embodiments may be subject to any suitable conditions or rules that determine whether the data can be transmitted. For example, a device may first check to verify (1) that a device designated to receive the data is within range; (2) that both devices have sufficient power to send the request and receive the data; (3) that the receiving device has sufficient space in its memory to store the data, and/or whether any other suitable condition is met.

User access to the server 1550 may be controlled via an authentication process. In various embodiments, authentication can be authorized using authentication tokens. In various embodiments, authentication tokens may comprise either simple or complex text strings or data values indicating an account number or other user identifier that can be matched against an internal database by the central server 1550. Alternatively, authentication tokens may comprise encoded passwords or other indicia that assert that the entity for which authentication is requested is genuine. Generation of an authentication token may be accomplished using alternative methods such as entry of a user identifier, PIN, or password by the user after being prompted to do so. Alternatively, a biometric measurement of the user could be obtained and the measurement rendered into a digital representation. Once generated, for security purposes the authorization token may be secured by encrypting the token, digesting and encrypting the digest of the token, or cryptographically hashing the token before transmission to the requesting entity. When authentication tokens are created, the originating component of the token may create a certification of validity through at least one of the following methods: (1) encrypting the token with a private key associated with the token originator; (2) encrypting the token with a public key associated with the token requester or destination; (3) generating a digest of the token (through a method such as a hashing algorithm discussed above) and optionally encrypting the hashed digest with the token originator's private key, or (4) providing an authentication code as at least part of the token (such as a cryptographically hashed password) that may be compared to previously stored values. When a component receives the token along with any encrypted or cleartext certification data, the component may determine the access is valid by (1) attempting to decrypt an encrypted token with the alleged originator's public key; (2) attempting to decrypt an encrypted token with the alleged originator's public key; (3) attempting to decrypt an encrypted digest with the alleged originator's public key, and comparing the result to a hashed value of the token, pin, code, or password, or (4) comparing a cryptographically hashed password for the alleged originator to known pre-stored values, and if a match is found, authorization is granted.

User Computing Device 1560

In FIG. 21, a user computing device 1560 can communicate with any of the other components in system 1500 via network 1540. The user computing device 1560 may include a personal computer or a mobile computing device, such as a laptop computer, a mobile wireless telephone, tablet computer, smartphone, or a personal digital assistant (PDA).

A user can use computing device 1560 to view, in real-time or near-real-time, the status of any of the components of a system, such as the components shown in FIGS. 21 and 22. The computing device 1560 may also be used to send commands to control such components or to the monitored asset, as well as to view reports showing data from the sensor devices 1510, or to analyze the data to generate metrics regarding the status of the monitored asset. Data can be provided to or received from a user of the computing device 1560 in a machine-readable format. The computing device 1560 may be configured to send, receive, and process machine-readable data can in any standard format (such as a MS Word document, Adobe PDF file, ASCII text file, JPEG, or other standard format) as well as any proprietary format. Machine-readable data to or from the user interface may also be encrypted to protect the data from unintended recipients and/or improper use.

The server 1550 or user computing device 1560 may include any number and type of processors to retrieve and execute instructions stored in the memory storage device of the server to control its functionality. The server 1550 may include any type of conventional computer, computer system, computer network, computer workstation, minicomputer, mainframe computer, or computer processor, such as an integrated circuit microprocessor or microcontroller in accordance with the present invention. The server 1550 or computing device 1560 operating in conjunction with the present invention may include any combination of different memory storage devices, such as hard drives, random access memory (RAM), read only memory (ROM), FLASH memory, or any other type of volatile and/or nonvolatile memory. The server 1550 may include an operating system (e.g., Windows, OS2, UNIX, Linux, Solaris, MacOS, etc.) as well as various conventional support software and drivers typically associated with computers. Software applications stored in the memory may be entirely or partially served or executed by the processor(s) in performing methods or processes of the present invention.

The server 1550 or computing device 1560 may also include a user interface for receiving and providing data to one or more users. The user interface may include any number of input devices such as a keyboard, mouse, touch pad, touch screen, alphanumeric keypad, voice recognition system, or other input device to allow a user to provide instructions and information to other components in a system of the present invention. Similarly, the user interface may include any number of suitable output devices, such as a monitor, speaker, printer, or other device for providing information to one or more users.

Any of the components in FIGS. 21 and 22 can be configured to communicate with each other (or with other additional systems and devices) for any desired purpose. For example, the server 1550 or user computing device 1560 may be used to upload software or firmware updates to sensor device 1510 or other component, provide or update encryption keys, and to perform diagnostics on any of the components in systems 1500 or 1600. Any computer system may be configured (i.e., using appropriate security protocols) to communicate instructions, software upgrades, firmware upgrades, data, and other information with components via network 1540. In various embodiments, data received from the sensor devices 1510 can be processed into a report and electronically provided (i.e., via email) to multiple users in a ubiquitous data format such as Portable Document Format (PDF). Such reports can be created at the request of a user or generated automatically at predetermined times or in response to the occurrence of an event (such as a detected problem with a monitored asset).

Any combination and/or subset of the elements of the methods depicted herein may be practiced in any suitable order and in conjunction with any system, device, and/or process. The method described herein can be implemented in any suitable manner, such as through software operating on one or more systems or devices, including the systems described in FIGS. 21 and 22.

As previously mentioned, the sensor device can be configured to have one or more sensors connected. In accordance with various embodiments, the types of sensors that could be connected to the sensor device include, but are not limited to, a vibration sensor such as an accelerometer, a pressure transducer such as piezoelectric transducer, a total dissolved solid (TDS) sensor such as an electrical conductivity meter, a hydrocarbon sensor such as an e-nose sensor, a temperature sensor such as a thermocouple, thermistor, or infrared thermometer, and a wind speed sensor such as an anemometer.

In accordance with various embodiments, a sensor device can comprise at least one sensor operatively coupled to a controller and a wireless communication device coupled to the controller. The controller can be configured to receive a measured input from the at least one sensor. The wireless communication device can be configured to communicate with a central server. Furthermore, the wireless communication device can transmit data to the central server at regular intervals. In various embodiments, the wireless communication device further transmits data to the central server in response to the measured input exceeding a predetermined threshold. Moreover, in various embodiments, the sensor device further comprises a power source, such as solar power, thermal power, battery power, and/or wind power.

The sensor device can be used in a variety of applications, such as the oil and gas wells as mentioned above. For example, the sensor device can be coupled to a fluid holding tank. The at least one sensor can be a volume sensor configured to determine the fluid volume in the fluid holding tank. More specifically, the volume sensor can be a pressure transducer located near the bottom of the fluid holding tank. The data obtained from the volume sensor can be used to determine a fill rate of the fluid holding tank based on a rate of volume change.

Furthermore, in one embodiment, the sensor can be a flow meter sensor configured to determine the flow rate into in the fluid holding tank. In another embodiment, the sensor can be a total dissolved solids (TDS) sensor configured to monitor fluid composition in the fluid holding tank. In yet another embodiment, the sensor can be an infrared thermal monitor configured to monitor flumes from a tank vent of the fluid holding tank, wherein the infrared thermal monitor can be configured for sensing volatile organic compounds. In another embodiment, the sensor can be an air quality sensor configured to measure air pollutants surrounding the fluid holding tank. In addition, in various embodiments the sensor device can be one of a plurality of sensor devices in a remote sensing system. Each of each of the plurality of sensor devices can be configured to communicate with at least one other sensor devices.

Predictive Analysis Using Vibration Data:

In accordance with various embodiments, a sensor device can be connected to a vibration sensor, such as an accelerometer. The sensor can be attached to various parts of an engine or machine and measure the ongoing vibrations. By way of example, the engine or machinery parts that vibrate include valves, bearings, crank shaft, camshaft, rocker arm, radiator fan, fly wheel, hydraulic pump, alternator, turbo, and fuel pump. Using an engine mount as an example, in various embodiments, the sensor device can obtain a baseline of vibration data when the engine is operating. This baseline can be measured manually prior to installation of the sensor devices, and/or obtained after the installation of the sensor devices. Furthermore, a software program can be executed to analyze the vibration patterns in comparison to the baseline vibration patterns. The software program may be installed on the sensor device, the coordinator, or the central server. Furthermore, the software program can search for vibration patterns with known timing, either from the baseline or from a library of specific component vibration patterns in order to determine potential sources of vibration patterns. In various embodiments, the software program analyzes the vibration data looking for changes in pattern for predictive analysis.

If multiple sensor devices are used on an engine, the vibration data from the multiple sensor devices can be used to triangulate the source of the change in the vibration pattern. The magnitude of change in vibration pattern can be used to triangulate the source of the disruption. This can provide an indication of which component of the engine may be failing and allow repair prior to a major failure. Moreover, an oil and gas company most likely implements the same type of machinery in multiple locations. Since the machinery is the same, the data from one location can be helpful in the diagnostics of the machinery in another location. In various embodiments, the sensing system can store the vibration data from multiple engines, and compare the change in vibration data to similar changes that occurred on other engines. This type of learning by the sensing system can provide additional information for diagnostics, such as an expected failure timeframe for the specific component. For example, if the change in vibration data indicates that a bearing may be beginning to fail, the system can provide an expected timeframe for the bearing's failure based on the data gathered from a similar bearing's failure.

Flow Rate:

In accordance with various embodiments, a flow rate into a fluid holding tank can be determined by a pressure sensor. The pressure sensor can be located at or near the bottom of the fluid holding tank, and can sense whether the pressure of the fluid is increasing, decreasing, or remaining constant. A change in the pressure data can be used to determine the flow rate of fluid into, or out of, the fluid holding tank. The flow rate data can be useful for different things. For example, a negative flow rate indicates that the fluid in a holding tank is being drained. In various embodiments, if the tank draining doesn't match a scheduled removal, this can trigger an alert that the fluid holding tank has a leak or that someone may be stealing the fluid.

Similarly, a positive flow rate can be correlated to production of the producing well. Simply that a high flow rate indicates high output from the well. Furthermore, the pressure sensor can take several data points, the flow rate can be tracked and more accurately show the output of a well. Measuring a well's flow rate in approximately real-time, in terms of minutes, increases the accuracy of a well's expected output. The wells may have short spikes of output or “burps” that distort a calculated flow rate if only measuring a well's output on a monthly basis.

Furthermore, in various embodiments, the flow rate data can be used to increase the confidence levels in production decline analysis. In a typical analysis, the production volume of a well may be recorded on a monthly basis. Using a sensor device, the flow rate, and hence production volume, of a well can be recorded at intervals of minutes. More continual monitoring, and enhanced accuracy, of the flow rate results in a production decline analysis curve with a higher confidence level in comparison to the current measurement methods.

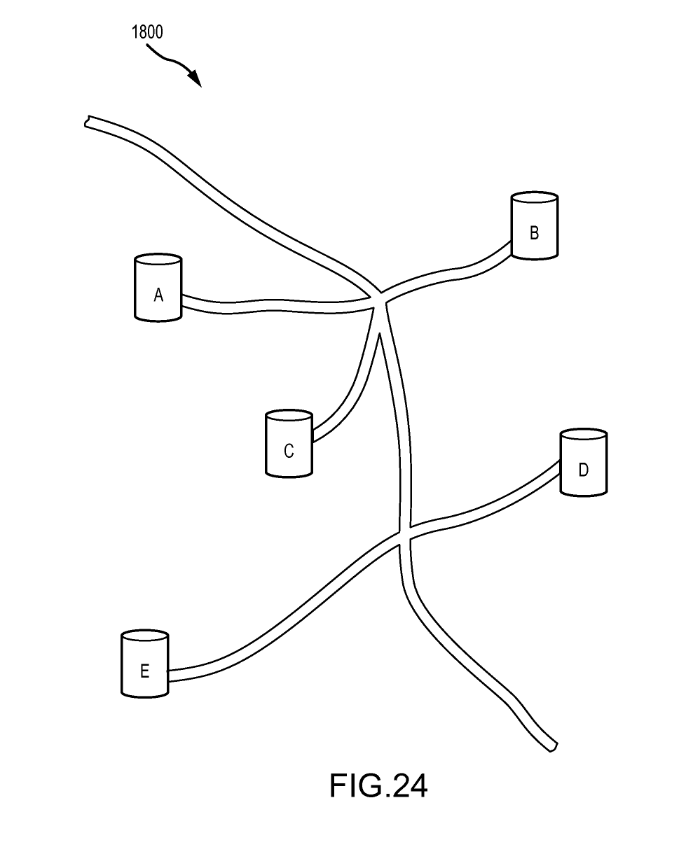

Another use of flow rate data can be determining when a tank needs to drained. In a field of tanks, this information can be used to determine an efficient tanker truck routing for draining the tanks In various embodiments and with reference to FIG. 24, a natural resource well field 1800 can include several holding tanks (designated A-E), each of which may have a different holding capacities and different fluid amounts being stored. The tanks with the least amount of time until being full can be given priority, and tanks that have a longer time until being full can be scheduled for a later stop. By correctly prioritizing the tanks and not checking the tanks that do not need to be drained, the truck routing will become more efficient, both in terms of time and number of miles driven. For example, flow rate data may indicated that holding tanks A-C are nearing capacity but holding tanks D-E still have low levels. The routing system can instruct trucks to proceed to drain tanks A-C but not check on nearby tanks D-E. This routing saves the driving distance and time it would take to check holding tanks D-E. Furthermore, the flow rate data can be used to determine the number of tanker trucks needed to carry out the fluid draining The routing system can determine the fluid volume to be drained and instruct the appropriate number of tanker trucks to proceed to the appropriate holding tanks This additional determination can save the driving distance and time of unnecessary tanker trucks.

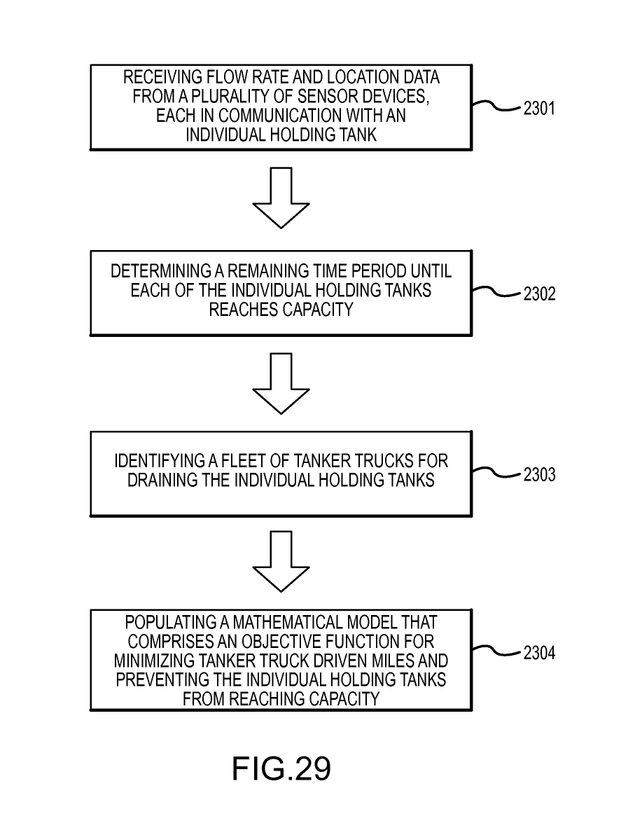

Accordingly, and with reference to FIG. 29 an exemplary logistics method can comprise receiving flow rate data from a plurality of sensor devices 2301, wherein each of the plurality of sensor devices can be in communication with an individual holding tank, wherein the data comprises a flow rate of the individual holding tanks, and wherein the data identifies the individual holding tank locations. The exemplary logistics method can also comprise determining a remaining time period until each of the individual holding tanks reaches capacity based on the flow rate and a remaining capacity of the individual holding tanks 2302, identifying a fleet of tanker trucks for draining the individual holding tanks 2303, and using the data to populate a mathematical model that comprises an objective function for minimizing tanker truck driven miles and preventing the individual holding tanks from reaching capacity 2304. The exemplary logistics method can further comprise determining a prioritized order of draining the holding tanks in the system based in part on the remaining time period of the individual holding tank.

In various embodiments, each of the plurality of sensor devices can comprise at least one sensor operatively coupled to a controller, wherein the controller can be configured to receive a measured input from the at least one sensor, and a wireless communication device coupled to the controller, wherein the wireless communication device can be configured to communicate with a central control system. The at least one sensor can be at least one of a flow meter and a pressure transducer.

In various embodiments, a logistics system can comprise a plurality of sensor devices providing data, wherein each of the plurality of sensor devices can be in communication with an individual holding tank. The data can comprise flow rates of the individual holding tanks, and can identify the individual holding tank locations. The logistics system can also include a capacity module configured to determine the time remaining until each of the individual holding tanks reaches capacity based on the flow rate and remaining capacity of the individual holding tanks Furthermore, the logistics system can also include an identification module configured to identify a fleet of tanker trucks for draining the individual holding tanks, along with a processor implementing a mathematical model populated by the data. The mathematical model can comprise an objective function for minimizing tanker truck driven miles and preventing the individual holding tanks from reaching capacity. The order of draining the tanks in the system can be determined in part by whether a first individual holding tank is closer to overflowing than a second holding tank.

Total Dissolved Solids Monitoring

In accordance with various embodiments, an electrical conductivity meter can be used to measure the conductivity of the fluid in a holding tank, thereby providing the concentration level of solids in the fluid and acting as a total dissolved solids (TDS) sensor. The electrical conductivity meter can be configured to measure a salt solution percentage of the stored fluid. In various embodiments, the electrical conductivity meter can be located near, or at, the input valve of the holding tank in order to measure the levels of the incoming fluid. In addition, in various embodiments, the sensor device can comprise a controller operatively coupled to the total dissolved solids (TDS) sensor and configured to receive the TDS data from the TDS sensor; and a wireless communication device coupled to the controller and configured to communicate with the central server. In various embodiments, the sensor device can be one of a plurality of sensor devices in a monitoring system. The TDS data can be transmitted from the sensor device to the central control system in real-time or in batch format. In addition, TDS level monitoring data can be correlated to multiple concepts, such a quality monitoring, well lifespan predictive analysis, and efficient by-product disposal.

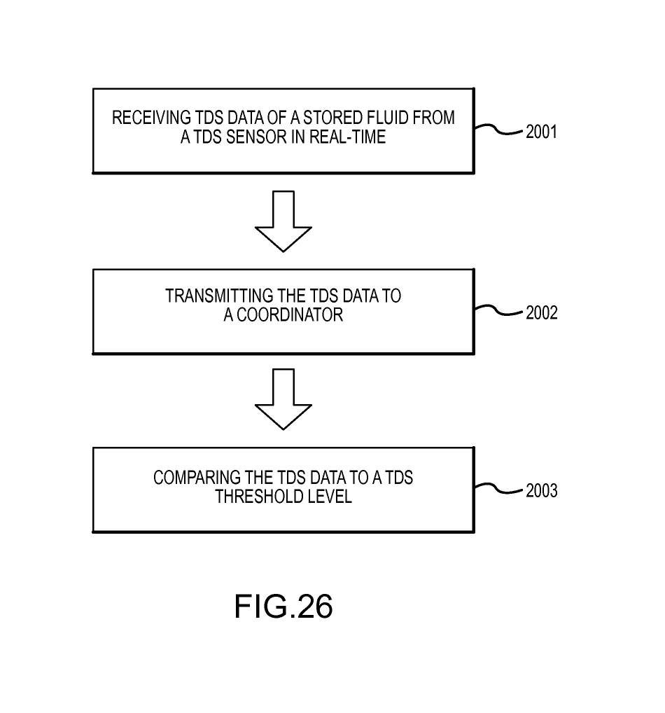

With respect to quality monitoring, a quality monitoring system can comprise a sensor device configured to receive TDS data of a stored fluid from a TDS sensor in real-time; and a central server configured to receive the TDS data from the sensor device. In various embodiments and with reference to FIG. 26, a quality monitoring method can comprise receiving, by the sensor device, TDS data of a stored fluid from a TDS sensor in real-time; transmitting, by the sensor device, the TDS data to the central control system; and comparing the TDS data to a TDS threshold level. In various embodiments, the quality monitoring method can further comprise notifying, by the sensor device, the central control system in response to the TDS data exceeding the TDS threshold level. The TDS threshold level can be set by a government agency, or may be set based on historical data.

The sensor device and TDS readings can be used in a variety of environments. For example, the stored fluid can be water by-product produced by a fracking well, which will undergo filtration, disposal, or reuse depending on the TDS level. The sensor device and readings can also be part of a water treatment facility, in which TDS levels are used determine the treatment process and/or the effectiveness of the treatment. Further, the TDS sensor and sensor device can be implemented in any factory or production facility that produces a fluid product or handles fluid by-products.

In another embodiment, the TDS sensor and sensor device can be implemented for water run-off monitoring, especially in remote areas. This can be useful for agriculture environments or industrial environments. For example, multiple sensor devices can be placed along a river bank and be solar powered. Each sensor device can take measurements for specific chemicals or pollutants. The sensor data can be transmitted and analyzed as described herein, and notice given if threshold levels are exceeded. The sensor data can also be used to determine whether an increase in chemical levels occurs at a specific section of the river, thereby assisting in narrowly the likely source of an increase.

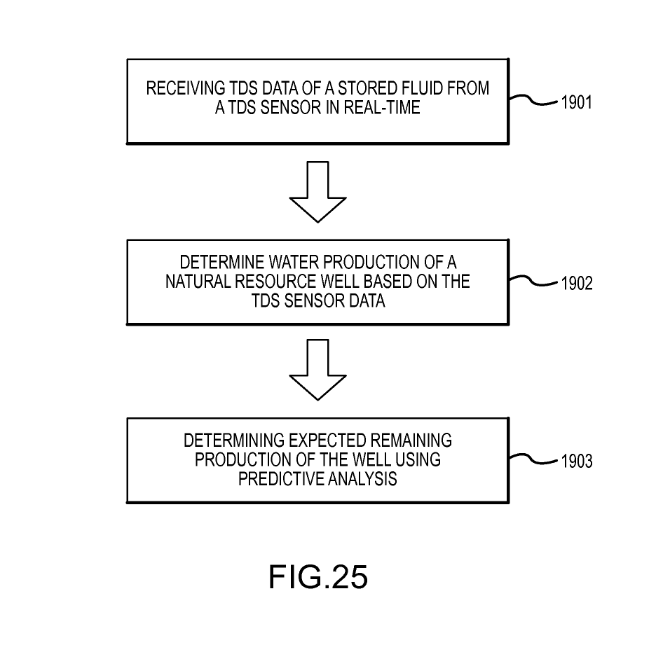

With respect to oil and natural gas wells, the composition of the output varies over the lifespan of the well. Oil wells will typically product fluid with a higher concentration of TDS towards the end of the well's lifespan. In accordance with various embodiments, TDS levels can be correlated to the lifespan of an oil or natural gas well. The TDS levels, specifically the change and value of the TDS levels, can be compared to historical data to predict the expected remaining lifespan of the oil or natural gas well. Accordingly, a holding tank monitoring system can comprise a sensor device configured to receive TDS data of a stored fluid from a TDS sensor in real-time. In various embodiments, the TDS sensor can be located at a top of a holding tank storing the stored fluid and/or near an input the holding tank. Further, the TDS sensor data can be used to determine a water percentage of the production of a natural resource well, and predictive analysis can be used to determine expected remaining production of the well. The stored fluid can be water by-product produced by a fracking well. Similarly, in various embodiments and with reference to FIG. 25, a holding tank monitoring method can comprise receiving, by the sensor device, TDS data of a stored fluid from a TDS sensor in real-time 1901, determining water production of a natural resource well based on the TDS sensor data 1902, and determining expected remaining production of the well using predictive analysis 1903, using historical data.

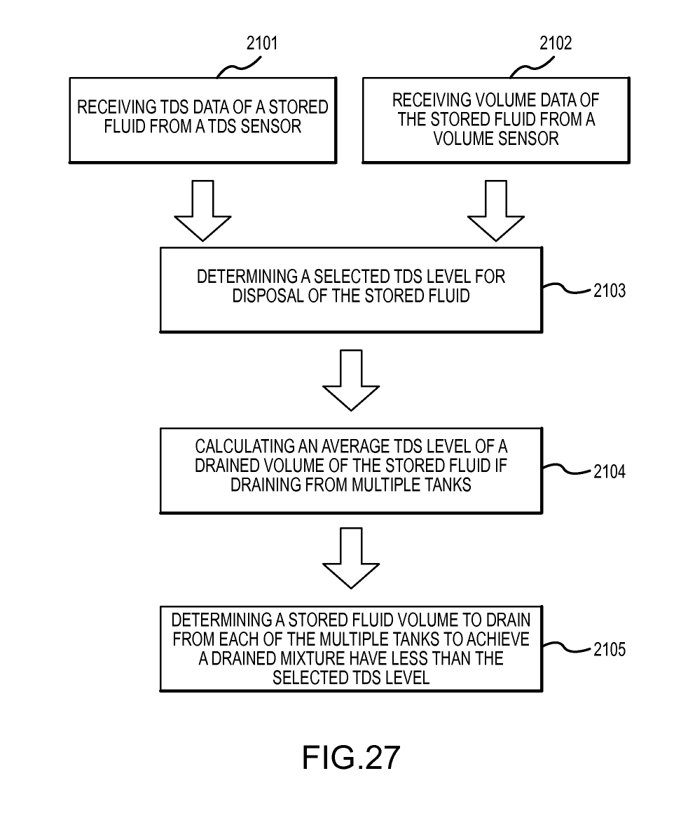

In addition to the uses mentioned above, water by-product disposal can also be improved using similar data. For example, the disposal of fracking fluid can be regulated based on the contaminant level of the fluid. Fluids with higher contaminant levels require more treatment, and are therefore more expensive when disposing. In addition, the processing or disposal areas may be different depending on the type of processing, which impacts where a driver should take the tanker truck when hauling the fluid. In various embodiments, the TDS level data can be used to inform a driver of the TDS level of a tank that is being drained and where to transport the tank for proper disposal. Moreover, in various embodiments, the tank fluid can be proportionally drained from multiple tanks into a single tanker truck, using the TDS level data, and resulting in a predetermined TDS level of the combined fluid. More specifically and with reference to FIG. 27, an exemplary method of selective holding tank draining can comprise receiving, by a sensor device, TDS data of a stored fluid from a TDS sensor 2101; receiving, by the sensor device, volume data of the stored fluid from a volume sensor 2102; determining, by a central control system, a selected TDS level for disposal of the stored fluid 2103; calculating an average TDS level of a drained volume of the stored fluid if draining from two or more tanks 2104; and determining a stored fluid volume to drain from each of the two or more tanks to achieve a drained mixture have less than the selected TDS level 2105. In other words, a driver can be provided instructions as to which tank or tanks to drain and the quantity to drain from each tank. The instructions are based on the tanker truck having a resulting tank of fluid with a selected level of TDS. The driver can also be instructed as to where to deliver the resulting fluid for proper disposal in accordance with the selected TDS level. The selected TDS level can be one of a plurality of predetermined TDS levels, where the disposal requirements of the drained mixture can be determined by regulations corresponding to the plurality of TDS levels. The regulations related to disposal based on TDS levels may be set by a government agency. Furthermore, the volume of the drained mixture can be less than the capacity of a tanker truck.

Volatile Organic Compound Monitoring

Volatile organic compounds (VOC) are naturally present as fugitive gases in and around oil or natural gas wells. Some VOCs are toxic and may be dangerous above certain concentrations. In accordance with various embodiments, a VOC sensor device can be used to monitor the VOC levels from a well or tank. The VOC measurement data can measure levels of benzene, toluene, ethylbenzene, and xylenes. In various embodiments, the VOC sensor device can comprise a sensor located in proximity to a vent or junction of a well. The VOC measurement data can be used to calculate fugitive losses from the tank or well. In current practice, the amount of fugitive gases escaping from a vent is unknown. However, VOC monitoring the flume from a vent enables the determination of the amount of VOCs escaping in the flume. For example, 5% of the flume may be VOCs, which equates to a certain amount per minute. The VOC sensor device can monitor for various VOC concentration thresholds or changes in the VOC concentration. Furthermore, the resulting VOC data on the fugitive gases facilitates deciding the appropriate method of capturing the fugitive gases, namely by providing the amount and rate of fugitive gases escaping.

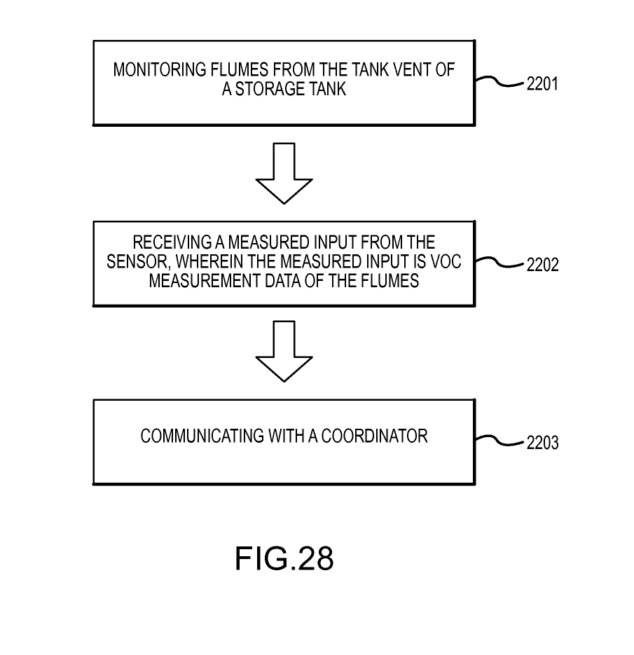

Furthermore, in various embodiments and with reference to FIG. 28, a method of volatile organic compound (VOC) monitoring can comprise monitoring, by a sensor located in proximity to a tank vent of a storage tank, flumes from the tank vent 2201; receiving, by a controller operatively coupled to the sensor, a measured input from the sensor, wherein the measured input can be VOC measurement data of the flumes 2202; communicating, by a wireless communication device coupled to the controller, with a coordinator 2203.

Further, in various embodiments, the VOC sensor device can also comprise a controller operatively coupled to the sensor, wherein the controller can be configured to receive VOC measurement data from the sensor, and a wireless communication device coupled to the controller, wherein the wireless communication device can be configured to communicate with a central control system. The central control system can be configured to analyze the VOC measurement data to determine if regulations are satisfied. The regulations can be set by a government agency. Moreover, the sensor device can be one of a plurality of sensor devices in a monitoring system.

Moreover, in various embodiments, the VOC sensor device can also comprise an infrared thermal monitor for monitoring temperature.

Air Quality Monitoring

Typically, natural gas wells are scattered throughout an area and at any given time one or more of the natural gas wells may have a leak. In the aggregate, small to moderate leaks from multiple wells combine to form fugitive gas levels that may exceed a government threshold. In the prior art, a sensor would measure for ozone, and if the ozone reading is above a threshold level, the system would assume a natural gas leak in the area. However, usually there is only a single sensor for a wide coverage area, and therefore the single sensor cannot determine the source of the leak, resulting in the entire coverage area being shut down until the gas levels dissipate or other corrections made.

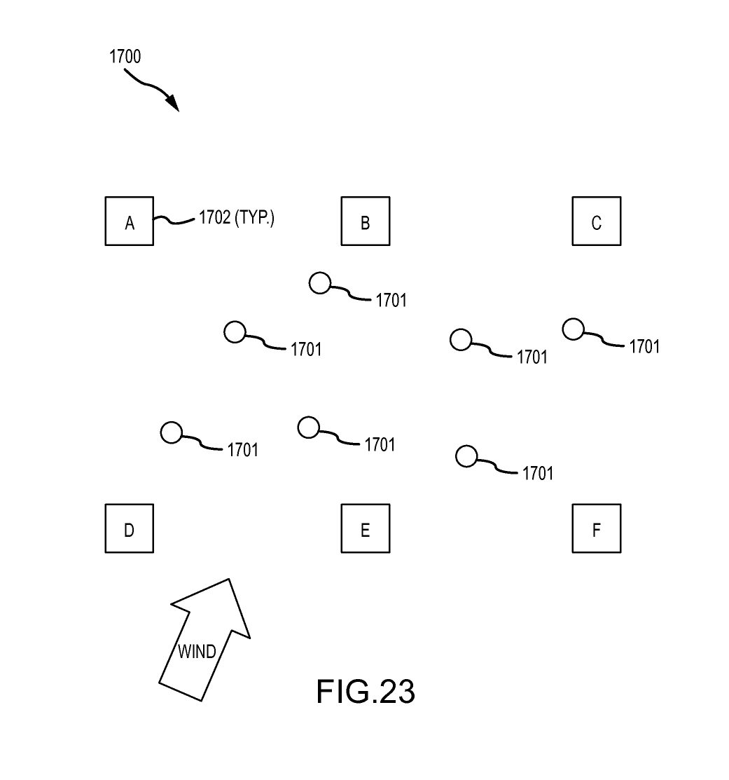

In accordance with various embodiments and with reference to FIG. 23, an air quality monitoring system 1700 can comprise multiple air quality sensor devices 1702 (designated as A-F) located throughout an area having natural gas wells 1701. Each of the air quality sensor devices 1702 can include a hydrocarbon sensor configured to measure fugitive gases, such as BTEX (benzene, toluene, ethylbenzene, and xylenes) in order to determine the air quality surrounding the natural gas wells. In accordance with various embodiments, a system of air quality sensor devices can be positioned in a grid system throughout a natural gas field. Furthermore, the sensor data can be collected and communicated in real-time. As used herein, “real-time” is defined to mean intervals measured in minutes. For example, the sensor data may be transmitted every 5 minutes, 10 minutes, 30 minutes, or the like.

In various embodiments, the air quality sensor device can include sensor types in addition to the hydrocarbon sensor, such as a temperature sensor for determining the ambient temperature at the hydrocarbon sensor. The ambient temperature can be an important factor in determining an acceptable threshold of fugitive gases. For example, higher temperatures may result in lower the threshold of fugitive gases, depending on the regulations. Furthermore, in various embodiments, the air quality sensor device can include an ultraviolet sensor for measuring ultraviolet levels. The air quality sensor device can also include an anemometer for measuring wind speed.

In various embodiments, any combination of the various sensors mentioned above can be connected to an air quality sensor device. The sensor device can be powered using a solar panel, a battery, or a combination of both. In various embodiments, the air quality sensors can be located on a pole so that it can be positioned about the ground, for example about 15 feet. Furthermore, the system can include an antenna, such as a Yagi antenna, for communicating the sensor data to a central system.

With reference to FIG. 23, the data from the various sensors can be used to determine if a natural gas well should be operated without exceeding an air quality threshold. The sensor data can be used to correlate the temperature, wind speed, ultraviolet levels, and fugitive gas levels with a dynamic threshold level. The advantages of the monitoring system include being able to narrow the area where the air quality threshold is being exceeded so that only a portion of the natural gas wells will be impacted, having earlier detection of an air quality issues since more sensors are deployed. For example, if air quality sensor device B has a higher hydrocarbon reading than air quality sensor device C at the illustrated wind direction, it can be determined that one of the natural gas wells 1701 within the area surrounded by sensor device A, B, D, E is most likely to be the cause of the increase hydrocarbon levels. In further embodiments, another advantage is being able to adjust natural gas wells operations at a more granular level. For example, if a certain area of the grid has high levels of fugitive gases, the system can compensate by implementing only a partial shutdown of the wells in that grid area rather than all the wells. The system can calculate, based on the sensor data, how many wells can be operational in that grid area without exceeding an air quality threshold.

An air monitoring array system can comprise a plurality of sensor devices arranged within a selected area, wherein the plurality of sensor devices can be configured to measure air pollutant levels in the selected area. In various embodiments, each sensor device can comprise at least one sensor operatively coupled to a controller, wherein the controller can be configured to receive a measured input from the at least one sensor; and a wireless communication device coupled to the controller, wherein the wireless communication device can be configured to communicate with a central control system. The central control system can be configured to determine if one or more portions of the selected area have air pollutant levels exceeding a predetermined threshold. The predetermined threshold may be set by a government agency. The at least one sensor can be a hydrocarbon sniffer, such as an e-nose sensor circuit as developed by NASA.

Valve Cover Power Unit

In accordance with various embodiments, a large thermoelectric generator (TEG) can be integrated into a valve cover of an engine. This can be accomplished by either removing a section of an already present valve cover and installing the TEG, or the TEG can be built into a valve cover and then used to replace an already present valve cover. In addition to valve covers, it is contemplated that the TEG can be integrated as part of any heat producing source. In addition to the TEG, a battery can also be included as an alternative energy source if the TEG is not sufficiently producing power (e.g., an engine is used as a heat source but is not currently operating). In various embodiments, the thermal electric core can be an array of multiple smaller thermal electric cores, or can be one large thermal electric core. The energy produced by a TEG can be linearly correlated to the surface area of the thermal electric cores in the TEG, so the different variations of the thermal electric core should produce approximately the same power.

In various embodiments, the valve cover can have a thermal barrier coating on the inside, outside, or both sides. The thermal barrier coating reflects heat, so that the inside of the valve cover is hotter than the outside of the valve cover. In one embodiment, the thermal barrier coating can be applied by spraying the material onto the valve cover. The increase in the temperature different between the inside and outside of the valve cover increases the amount of power generated by the TEG. This thermal barrier embodiment can be most beneficial in hot environments, such as the Middle East or other areas where the temperature on the outside of the valve cover can be high.

Furthermore, in various embodiments, the sensor device can vary its mode based on the power source. For example, if receiving power from the TEG device, then sensor device can have full functionality. However, if operating on battery power, most likely due to an issue with the TEG device, the sensor device can be configured to operate on partial functionality in order to draw less operating power. Additionally, in various embodiments, the sensor device can be provided an update to override the default partial functionality setting. An operator may choose to override and continue operating the sensor device at full functionality if the sensor device can be scheduled to be, or can be, serviced in the near future.

Data Transmission

Data collected from a sensor device 1510 or generated by any other device, such as the coordinator 1520, operating in conjunction may be transmitted to other systems, such as to central server 1550 for analysis. The data can be transmitted in any suitable manner, including using any of the wired or wireless communication methods and protocols described previously. Any amount of data can be transmitted in any manner. For example, data from the sensor device 1510 can be transmitted to another device (such as to coordinator 1520) as it is measured, or data can be stored (such as in a memory storage device in the sensor device 1510) for a period of time before being transmitted to another device. In some cases, for example, it may be more efficient to transmit blocks of data at once rather than initiating communication with another device each time data is available. Furthermore, the data can be transmitted at off-peak times when there are fewer transmissions occurring on a cellular or satellite network. In other cases, a device may be out of range or otherwise unavailable to receive the data. The data can also be stored for any desired length of time, and/or until a particular event occurs. For example, the device data could be stored until it can be verified that the receiving device and/or the data server 1550 have received the data, allowing the data to be retransmitted if necessary. Data can also be deleted when a data record exceeds a predetermined storage time, and/or the oldest data record can be deleted first after a predetermined storage size limit has been reached.

Data transmitted from the sensor devices 1510 may be validated to ensure it was transmitted properly and completely. The sensor device data may also be validated to ensure it was provided from a specific sensor device 1510 or group of sensor devices 1510 (i.e., associated with a particular asset being monitored). The data may also be validated to ensure that fields in the data correspond to predetermined values and/or are within certain thresholds or tolerances. Any number, code, value or identifier can be used in conjunction with validating the device data. For example, the data can be validated by analyzing a serial number, a device identifier, one or more parity bits, a cyclic redundancy checking code, an error correction code, and/or any other suitable feature.

In exemplary embodiments, various components (such as coordinator 1520, gateway 1530, and server 1550) may be configured to receive data directly or indirectly from a sensor device 1510, format a message based on the data, and transmit the formatted message to another system or device. This functionality may be implemented through software operating on any suitable mobile computing device and with any computer operating system.

Receipt of data from the sensor devices 1510 may be restricted only to authenticated devices operating as part of the system. Authentication can also prevent sensitive data from being broadcast and viewed by unintended recipients. Any device may be authenticated to verify the device can be able to receive, process, and/or transmit data. During authentication, the authenticated device or devices may also be remotely commanded, and such commands may include steps that configure devices to interoperate with components of the present invention. For example, but not by way of limitation, such steps may include the downloading of software applications, applets, embedded operating code, and/or data.

Devices can be authenticated in any manner. For example, devices can be authorized to receive data from one or more sensor devices 1510 using an authorization code. The authorization code can be any number, code, value or identifier to allow the receiving device to be identified as a valid recipient of the data. In various embodiments, the receiving device stores an authorization code and broadcasts the authorization code in response to a request for authorization. Unless the authorization code matches a code stored by the transmitter of the data (such as the sensor device 1510 itself or another transmission device), the data is not transmitted to the device.

In other exemplary embodiments, the coordinator 1520, gateway 1530, or other device receiving the data from the sensor device 1510 using a wireless network protocol (such as Bluetooth®) can be authenticated based on whether the receiving device advertises one or more services. In this context, advertised services reflect functions, utilities, and processes the receiving device can be capable of performing. The receiving device broadcasts indicators of this functionality, thus “advertising” them to other systems and devices. In such embodiments, unless the receiving device advertises a service that can be identifiable with the operation of the present invention (i.e., a process capable of broadcasting the sensor device 1510 data to the central server 1550, for example), the receiving device is not authenticated and thus the data is not transmitted to the device.

Data can be transmitted to components operating in conjunction with the present invention in any format. For example, data from the sensor device 1510 can be transmitted to the coordinator 1520 exactly as it is generated by the sensor unit 1650 of the sensor device 1510, or it can be reformatted, modified, combined with other data, or processed in any other suitable manner before being transmitted. For example, the data can be encrypted prior to transmission, and this encryption may occur at any stage in its transmission by the sensor device 1510 or retransmission by another device. Some or all of the data being transmitted may be encrypted. In some embodiments, a digest of the data may be encrypted, to digitally “sign” the data contents to verify its authenticity. For example, but not by way of limitation, this digest may be produced by providing the received data to a hashing algorithm such as the MD5 or SHA-1 Secure Hashing Algorithm as specified in National Institute of Standards and Technology Federal Information Processing Standard Publication Number 180-1.

In some embodiments, such as described for the system 1600 depicted in FIG. 22, a group of coordinators 1520 may be configured to relay communications amongst themselves when fewer than all coordinators 1520 are within communication range of a gateway 1530.

Commands from the Server

In addition to receiving and processing data from the sensor devices 1510 and other components operating in conjunction with embodiments of the disclosure, the server 1550 (or user computing device 1560 if desired) can transmit a command to control various functions of such components, the asset being monitored, or other systems and devices. Any number of commands of any type may be transmitted by the server 1550 to any suitable recipient. The command can be transmitted using the same variety of wired and wireless methods discussed previously. For example, the server 1550 may issue a command to control, reconfigure, and/or update a software application operating on the gateway 1530, coordinator 1520, and/or sensor device 1510.

The commands need not be sent directly to a device they are intended to control. For example, a command could be transmitted to a coordinator 1520, which in turn retransmits it (unmodified) to the appropriate sensor device 1510. Alternatively, the coordinator 1520 could receive a command from the server 1550, analyze the command, and then transmit an appropriately formatted command tailored to the specific sensor device 1510 to be controlled. In this manner, the server 1550 need not be able to generate a command for each and every specific device it wishes to control, rather, it can send a command appropriate to a class of sensor devices (i.e. those with vibration sensors) and the coordinator 1520 can appropriately translate the command to control the sensor device 1510. The commands from the server 1550 can initiate/run diagnostic programs, download data, request encryption keys, download encryption keys, and perform any other suitable function on devices operating in conjunction with systems and methods of the present invention.

In any system where commands can be sent remotely, security is always a concern, especially when a wireless implementation may provide an entry vector for an interloper to gain access to components, observe confidential data, and control assets such as expensive oil and gas engines/pumps. Embodiments of the present invention provide for enhanced security in a remote command system while still allowing flexibility and minimal obtrusiveness.

In one embodiment, a command received by any of the components in FIG. 21 or 22 may be authenticated before the command is either acted upon by the destination component, or forwarded to another component in the system. Authentication may be directed to determining (1) whether the command came from a trusted or authorized source and (2) that the recipient is actually the intended recipient of the command. In one implementation, source command authentication can be achieved by determining whether the origin of the command is a trusted component or server, and one way to accomplish this determination can be analyzing whether a command is properly digitally signed by the originator or some other authentication information can be provided that assures the recipient component that the message or command is authentic and the recipient component is actually the intended recipient. In an alternate implementation, destination command authentication can be accommodated by examining the contents of the message or an authorization code to determine the intended recipient, or alternatively decrypting the command or a portion of the command to verify the intended recipient.

When commands are created by a command originator, the originator may allow a recipient to verify the authenticity and/or validity of the command by at least one of the following methods: (1) encrypting the command with a private key of the command originator; (2) generating a digest of the command (through a method such as a hashing algorithm discussed above) and optionally encrypting the hashed digest with the command originator's private key, or (3) utilizing a symmetric encryption scheme providing an authentication code (such as a cryptographically hashed password) that can be compared to previously stored values. When a system component receives the command along with any encrypted or cleartext certification data, the component may determine the command is valid by: (1) attempting to decrypt an encrypted command message with the alleged originator's public key, (2) attempting to decrypt an encrypted digest with the alleged originator's public key, and comparing the result to a hashed value of the command, or (3) comparing a cryptographically hashed password for the alleged originator to known pre-stored values, and if a match is found, authorization can be granted. As an additional step, if the command were optionally encrypted using the intended provider's public key, then only the recipient is capable of decrypting the command, ensuring that only the truly intended recipient devices were being issued commands, and not an unintended third party. For example, authenticating the command may comprise decrypting at least part of the command using at least one of: a public key associated with the server 1550; a private key associated with a sensor device 1510; and a private key associated with the sensor device 1510.

Systems and devices operating in accordance with aspects of the present invention may implement one or more security measures to protect data, restrict access, or provide any other desired security feature. For example, any device operating in conjunction with the present invention may encrypt transmitted data and/or protect data stored within the device itself. Such security measures may be implemented using hardware, software, or a combination thereof. Any method of data encryption or protection may be utilized in conjunction with the present invention, such as public/private keyed encryption systems, data scrambling methods, hardware and software firewalls, tamper-resistant or tamper-responsive memory storage devices or any other method or technique for protecting data. Similarly, passwords, biometrics, access cards or other hardware, or any other system, device, and/or method may be employed to restrict access to any device operating in conjunction with the present invention.

Some exemplary embodiments of the invention are as follows.

### Example Set 1

1) A system comprising:
   a. a sensor device, the sensor device comprising:
      i. a processor;
      ii. a transceiver coupled to the processor;
      iii. a sensor coupled to the processor and configured to measure a characteristic associated with a monitored asset; and
      iv. a non-transitory memory coupled to the processor and storing instructions executable by the processor for:
         receiving data from the sensor; and
         transmitting the received data via the transceiver; and
   b. a coordinator configured to receive the transmitted data.
2) The system of example 1, wherein the transceiver is configured to transmit data using one or more of: a Zigbee protocol, a Wibree protocol, an IEEE 802.11 protocol, an IEEE 802.15 protocol, an IEEE 802.16 protocol, an Ultra-Wideband (UWB) protocol, an Infrared Data Association (IrDA) protocol, a Bluetooth protocol, and combinations thereof.
3) The system of example 1, wherein the transceiver is configured to transmit data through a wired connection selected from the group consisting of an optical fiber connection, a tip and sleeve (TS) connection, a tip, ring, and sleeve (TRS) connection, a tip, ring, ring, and sleeve (TRRS) connection, a serial peripheral interface bus (SPI) connection, a universal serial bus (USB) connection, an RS-232 serial connection, an Ethernet connection, a FireWire connection, and combinations thereof
4) The system of examples 1, further comprising a gateway configured to receive the data transmitted from the coordinator, wherein the gateway transmits the data received from the coordinator through a network.
5) The system of example 4, wherein the network comprises one or more of a local area network (LAN), wide area network (WAN), wireless mobile telephony network, General Packet Radio Service (GPRS) network, wireless Local Area Network (WLAN), Global System for Mobile Communications (GSM) network, Personal Communication Service (PCS) network, Advanced Mobile Phone System (AMPS) network, a satellite communication network, and combinations thereof
6) The system of any of examples 1-5, further comprising a server coupled to the network, the server configured to receive the data from the gateway.
7) The system of example 6, wherein the server is configured to analyze the data and determine a metric.
8) The system of example 1, wherein the sensor device is configured to transmit the data intermittently to the coordinator.
9) The system of any of examples 1-8, wherein the coordinator is configured to transmit the data intermittently to the gateway.
10) The system of any of examples 4-7, wherein the gateway transmits data intermittently via the network.
11) The system of any of examples 1-10, further comprising a plurality of sensor devices.
12) The system of any of examples 1-11, further comprising a plurality of coordinator devices.
13) The system of any of examples 1-12, further comprising a plurality of gateways, wherein each gateway is configured to receive the data transmitted from the coordinator.
14) The system of any of examples 1-13, wherein a first plurality of sensor devices communicate with a first coordinator device, and a second plurality of sensor devices communicate with a second coordinator device.
15) The system of example 14, wherein the first coordinator device is in communication with at least one gateway configured to receive the data from the coordinator.
16) The system of example 15, wherein the first coordinator device is configured to relay communications between the at least one gateway and the second coordinator device.
17) The system of any of examples 1-16, wherein the sensor device is mounted on an engine.
18) The system of any of examples 1-17, wherein the data received from the sensor is analyzed by the processor.
19) The system of example 1, wherein the sensor device has a casing and the power source, processor and transmitter are inside the casing.
20) The system of example 19, wherein the sensor device is mounted on an engine.
21) The system of any of examples 1-20, comprising a plurality of sensor devices mounted on an engine.
22) The system of example 21, wherein each sensor device monitors the function of an individual component of the engine.
23) The system of example 22, wherein the component is selected from the group consisting of: a crankshaft, a valve, a cylinder, a bearing, a belt, a wheel, and combinations thereof
24) The system of example 21 wherein the engine is a compressor.
25) The system of any of examples 1-23, wherein the engine has a valve cover and an opening is formed in the valve cover adjacent each cylinder for mounting the sensor device.
26) The system of any of examples 1-25, wherein the sensor device further includes a power source.
27) The system of example 26, wherein the sensor device further includes a system for recharging the power source.
28) The system of example 27, wherein the sensor device is mounted on an engine having a valve cover with an opening, and the system for recharging the power source includes a heat pipe that extends through the opening in the valve cover.
29) The system of example 28, wherein the sensor device includes a case that contains the power source, the processor and at least part of the heat pipe.
30) The system of example 28, wherein the system for recharging the power source further includes a thermal energy generator that receives heat from the heat pipe.
31) The system of any of examples 1-30, wherein the sensor is configured to measure a characteristic selected from the group consisting of: temperature, pressure, flow, vibration, strain, an electrical parameter, an atmospheric condition, sound, a chemical, radiation, position, force, movement, and combinations thereof
32) The system of any of examples 1-31, wherein the sensor device transmits the data to the coordinator at regular intervals.
33) The system of any of examples 1-32, wherein the sensor device is configured to:
   (a) analyze the data from the sensor to detect a condition associated with the monitored asset; and
   (b) transmit the data to the coordinator when the condition is detected.
34) The system of example 33, wherein the detected condition is selected from the group consisting of: a possible failure of a mechanical component of the monitored asset, a hazardous level of a substance, a potentially-hazardous weather event, a measured sensor reading beyond a predetermined threshold, and combinations thereof.
35) The system of any of examples 1-33, wherein the sensor device further comprises a communication interface coupled to the monitored asset.
36) The system of example 35, wherein the sensor device is configured to receive data from a computer system coupled to the monitored asset.
37) The system of example 36, wherein the monitored asset includes an engine, wherein the computer system is an on-board computer for the engine, and wherein the on-board computer is coupled to one or more on-board sensors.
38) The system of example 35, wherein the sensor device is configured to control all or part of the functionality of the monitored asset.
39) The system of example 38, wherein the monitored asset includes an engine, and wherein the sensor device is configured to control one or more of: power to the engine, an operating speed of the engine, a fuel mixture provided to the engine, and combinations thereof
40) The system of any of examples 1-39, wherein the monitored asset is an engine, wherein the sensor is configured to detect hydrocarbon, and wherein the sensor device is configured to:
   (a) analyze data from the sensor and detect an elevated level of hydrocarbon; and
   (b) send an alert, via the transceiver, regarding a possible exhaust leak associated with the engine.
41) The system of any of examples 1-40, wherein the sensor device is configured to perform a diagnostic on the sensor to determine whether the sensor is functioning properly.
42) The system of any of examples 1-41, wherein the sensor device is configured to perform a diagnostic on itself to determine whether the sensor device is functioning properly.
43) The system of any of examples 1-42, wherein the sensor device is configured to generate an alert, via the transceiver, in response to a determination that one or more of the sensor and sensor device is not functioning properly.
44) The system of any of examples 1-43, wherein one or more of the sensor device, the sensor, the coordinator, and the gateway is configured to wirelessly receive and install a software update.

### Example Set 2

1. A method for monitoring the functioning of a machine, the method comprising:
   (a) measuring the temperature of the machine;
   (b) converting the measured temperatures into electronic data;
   (c) storing the measured temperatures and creating a database of measured temperatures and the time each of the temperatures was taken;
   (d) establishing a communications link between a first transmitter and a first receiver;
   (e) establishing a communications link between the first transmitter and the database;
   (f) transmitting all or part of the database to the first receiver from the first transmitter; and
   (g) analyzing the all or part of the transmitted database to monitor the machine's functionality.
2. The method of example 1 that further includes the step of analyzing the database to establish a standard operating temperature range for the machine.
3. The method of example 1 wherein the first receiver is connected to a second transmitter that transmits all or part of the database to a second receiver.
4. The method of example 2 wherein after the standard operating temperature range has been established, at least some of the temperatures measured thereafter are compared to the standard temperature operating range.
5. The method of example 2 wherein after the standard operating temperature range has been established, all of the temperatures measured thereafter are compared to the standard temperature operating range.
6. The method of example 2 wherein after the standard temperature operating range has been established, the temperatures measured thereafter are compared to the standard temperature operating range on predetermined time intervals.
7. The method of any of examples 3-6 wherein when a temperature measured after the standard temperature operating range has been established exceeds a predetermined level, an alarm is transmitted, the alarm detectable by a user.
8. The method of any of examples 1-7 wherein the database is stored in a memory.
9. The method of example 8 wherein the memory is accessed by a controller.
10. The method of examples 3-9 wherein when a temperature measured after the standard temperature operating range has been established exceeds a predetermined level, a signal is sent to shut off the machine.
11. The method of example 10 wherein a signal is sent to shut down the engine after a plurality of temperatures exceeding the standard temperature operating range have been measured.
12. The method of example 11 wherein the plurality of temperatures exceeding the standard temperature operating range are measured over a predetermined time interval.
13. The method of example 12 wherein the predetermined time interval is five minutes or more.
14. The method of any of examples 3-13 wherein each temperature exceeding the standard temperature operating range is at least 15° C. above the standard temperature operating range.
15. The method of any of examples 1-14 wherein the machine is an engine and the temperature is measured for a plurality of the engine valves.
16. The method of any of examples 1-14 wherein the machine is an engine and the temperature is measured inside each cylinder of the engine.
17. The method of any of examples 1-14 wherein the machine is an engine and the temperature is measured inside each cylinder of the engine and for each set of valves for each cylinder.
18. The method of any of examples 15-17 wherein a signal is sent to lower the RPM of the machine after a temperature is measured that exceeds the standard temperature operating range.
19. The method of any of examples 1-17 wherein a signal is sent to lower the RPM of the machine after a plurality of temperatures have been measured over a predetermined time interval that exceed the standard temperature operating range.
20. The method of example 19 wherein the predetermined time is five minutes or more.
21. The method of example 7 wherein after receiving the alarm the user either (a) sends a signal to shut off the machine, (b) sends a signal to slow the RPM of the machine, or (c) takes no action.
22. The method of either example 7 or 21 wherein the user sends a communication to repair personnel.
23. The method of any of examples 9 or 26-30 wherein the controller receives software updates.
24. The method of example 1 wherein the database is resident in the controller of claim 9.
25. The method of example 7 wherein the alarm is transmitted by the first transmitter.
26. The method of example 9 wherein the controller continually accesses the memory.
27. The method of example 10 wherein the signal is sent by the controller of example 9.
28. The method of example 11 wherein the signal is sent by the controller of example 9.
29. The method of example 18 wherein the signal is sent by the controller of example 9.
30. The method of example 19 wherein the signal is sent by the controller of example 9.
31. The method of example 1-14 or 19-28 wherein the machine is an engine.

### Example Set 3

1. A method comprising:
   (a) measuring the vibration of one or more components of an engine;
   (b) converting the measured vibrations into electronic data;
   (c) storing the electronic data in a database;
   (d) establishing a communications link between a first transmitter and a first receiver;
   (e) establishing a communications link between a first transmitter and the database;
   (f) transmitting all or part of the database to the first receiver; and
   (g) analyzing the transmitted part of the database to monitor the engine's functionality.
2. The method of example 1 wherein the database is resident on a device attached to the engine.
3. The method of either of examples 1 or 2 wherein the vibration is measured using one or more accelerometers.
4. The method of any of examples 1-3 wherein the database is resident on a semiconductor.
5. The method of example 4 wherein the semiconductor is inside of a casing positioned on the engine.
6. The method of example 1 wherein the database is resident remote from the engine.
7. The method of any of examples 1-6 wherein the vibration of the one or more engine components is continuously measured.
8. The method of any of examples 1-7 wherein at least part of the database is analyzed to establish a standard vibration operating parameter for at least one of the one or more engine components for which the vibration is being measures.
9. The method of example 1 wherein the database is maintained at the first receiver.
10. The method of either of examples 1 or 4 wherein the first transmitter is an antenna.
11. The method of any of examples 1-10 that further includes a second transmitter in communication with the first receiver, the second transmitter for transmitting at least part of the database to a second receiver.
12. The method of any of examples 1-11 wherein the vibrations are measured for a plurality of the engine's cylinders.
13. The method of any of examples 1-10 wherein the vibrations are measured for each of the engine's cylinders.
14. The method of example 8 wherein after the standard vibrational operating parameter is established, each subsequent vibration measured is compared to the standard vibrational operating parameter to determine if the engine is functioning properly.
15. The method of example 8 wherein a standard vibrational operating procedure is established for each engine cylinder.
16. The method of any of examples 1-15 wherein the vibration of at least one of the valve covers is measured and stored.
17. The method of any of examples 1-16 wherein the vibration of at least one of the valve rocker arms is measured and stored.
18. The method of any of examples 1-17 wherein the vibration of the cam shaft is measured and stored.
19. The method of any of examples 1-18 wherein the vibration of the fly wheel is measured and stored.
20. The method of any of examples 1-19 wherein if any measured vibration exceeds a predetermined vibrational parameter, a signal is sent to either (a) shut down the engine, (b) slow the RPM of the engine, or (c) notify an operator or repair personnel.
21. The method of any of examples 1-19 wherein if any measured vibration exceeds a predetermined vibrational parameter for a predetermined time, a signal is sent to either (a) shut down the engine, (b) slow the RPM of the engine, or (c) notify an operator or repair person.

### Example Set 4

1. A valve cover for use on an engine, the valve cover for retaining a device that generates power for a system that monitors one or more of temperature, vibration, flow and chemical composition.
2. The valve cover of example 1 that is attached to the engine.
3. The valve cover of example 1 that replaces an original valve cover of the engine.
4. The valve cover of any of examples 1-3 wherein the device generates electricity by absorbing heat from the engine and transferring the heat to a thermal energy generator, which creates electricity.
5. The valve cover of any of examples 1-4 wherein the device includes a structure to dissipate heat.
6. The valve cover of example 5 wherein the structure to dissipate heat comprises one or more of: a plurality of metal rods and upwardly-extending metal fins.
7. The valve cover of example 3 wherein electricity is transferred to a second device via a wired connection.
8. The valve cover of any of examples 1-8 that is bolted onto the engine.
9. The valve cover of example 1 that powers a plurality of devices other than the one retained on the valve cover.
10. The valve cover of any of examples 1-10 wherein the device is mounted on a side of the valve cover.
11. The valve cover of any of examples 1-11 that is comprised of one or more of the group consisting of: plastic and metal.
12. The valve cover of any of examples 1-12 wherein the device has a heat pipe with a first end that extends into the valve cover, the first end for transferring heat to a thermal energy generator to generate electricity.

### Example Set 5

1. A system for recharging a battery, the system comprising:
   (a) a heat pipe having a first end, a second end and body therebetween, wherein the first end is configured to contact a heat source;
   (b) a thermal energy generator in contact with the second end;
   (c) a battery; and
   (d) a power converter in electrical contact with the thermal energy generator and in electrical contact with the battery, the converter converting a first electrical power received from the thermal energy generator into a second electrical power, the second electrical power transmitted to the battery to recharge it.
2. The system of example 1 wherein there is a conforming material between the second end of the heat pipe and the thermal energy generator, the conforming material conforming at least partially to the surface of the second end of the heat pipe and at least partially to the surface of the thermal energy generator so as to increase the surface area through which heat can be transmitted.
3. The system of example 2 wherein the conforming material is comprised of a graphite cloth.
4. The system of either of examples 2 or 3 wherein the conforming material is 1/32″ thick or less.
5. The system of any of examples 1-4 that further includes a container at the second end of the heat pipe, the container for retaining one or more of (a) the thermal energy generator, and (b) the conforming material.
6. The system of any of examples 2-5 wherein the heat pipe includes an insulating material covering at least some of the heat pipe in order to help prevent heat from dissipating from the heat pipe.
7. The system of any of claims 1-6 that further includes a PCB and the batter and power converter are on the PCB.
8. The system of example 6 wherein the insulating material is comprised of plastic.
9. The system of example 8 wherein the insulating material is a plastic sleeve that at least partially surrounds the heat pipe.
10. The system of either of examples 6 or 9 that further includes a casing that contains at least part of the heat pipe and the insulating material is inside of the casing.
11. The system of any of claims 1-10 wherein the second end of the heat pipe and the thermal energy generator are pressed together.
12. A system for recharging a battery, the system comprising:
   (a) a casing;
   (b) a power collection source external to the casing;
   (c) a processor inside of the casing, the processor in electrical communication with the power collection source;
   (d) a battery inside of the casing and in electrical communication with the processor;
   wherein the processor receives power from the power source and converts it into electricity that can recharge the battery and transmits the converted power to the battery.
13. The system of example 12 wherein the power collection source is a solar cell.

### Example Set 6

1. A sensor device comprising:
   at least one sensor operatively coupled to a controller, wherein the controller is configured to receive a measured input from the at least one sensor; and
   a wireless communication device coupled to the controller, wherein the wireless communication device is configured to communicate with a coordinator.
2. The sensor device of example 1, wherein the sensor device further comprises:
   a processor in communication with the at least one sensor and the wireless communication device; and
   a memory in communication with the processor and storing instructions executable by the processor for:
      receiving data from the at least one sensor; and
      transmitting at least a portion of the data to another sensor device via the wireless communication device.
3. The sensor device of examples 1-2, further comprising a power source for powering the sensor device.
4. The sensor device of example 3, wherein the power source comprises one or more of a battery and a capacitor.
5. The sensor device of example 4, wherein the power source comprises a battery, and the sensor device further comprises an energy harvester coupled to the power source for recharging the battery.
6. The sensor device of example 5, wherein the energy harvester includes one or more of a photovoltaic cell for collecting solar energy; a thermoelectric generator (TEG); and/or a piezoelectric vibrational energy harvester (PZEH).
7. The sensor device of examples 1-6, wherein the at least one sensor is coupled to a fluid holding tank.
8. The sensor device of example 7, wherein the at least one sensor is a volume sensor configured to determine the fluid volume in the fluid holding tank.
9. The sensor device of example 8, wherein the volume sensor is a pressure transducer located near the bottom of the fluid holding tank.
10. The sensor device of examples 8-9, wherein data from the volume sensor is used to determine a fill rate of the fluid holding tank based on a rate of volume change.
11. The sensor device of examples 7-10, wherein the at least one sensor is a flow meter sensor configured to determine the flow rate into in the fluid holding tank.
12. The sensor device of examples 7-11, wherein the at least one sensor is a total dissolved solids (TDS) sensor configured to monitor fluid composition in the fluid holding tank.
13. The sensor device of examples 7-12, wherein the at least one sensor is an infrared thermal monitor configured to monitor flumes from a tank vent of the fluid holding tank, wherein the infrared thermal monitor is configured for sensing volatile organic compounds.
14. The sensor device of examples 7-13, wherein the at least one sensor is an air quality sensor configured to measure air pollutants surrounding the fluid holding tank.
15. The sensor device of examples 1-14, wherein the wireless communication device is configured to transmit data from the at least one sensor to the coordinator.
16. The sensor device of example 15, wherein the wireless communication device is configured to transmit using at least one of a satellite communication network, a local area network (LAN), a wide area network (WAN), a wireless mobile telephone network, a General Packet Radio Service (GPRS) network, a wireless local area network (WLAN), a Global System for Mobile Communications (GSM) network, a Personal Communication Service (PCS) network, and an Advanced Mobile Phone System (AMPS) network.
17. The sensor device of examples 1-17, wherein the sensor device is one of a plurality of sensor devices in a monitoring system.
18. The sensor device of example 17, wherein each of the plurality of sensor devices is configured to communicate with at least one other sensor device of the plurality of sensor devices.
19. The sensor device of examples 1-18, wherein the wireless communication device transmits data to the coordinator at regular intervals.
20. The sensor device of examples 1-19, wherein the wireless communication device further transmits data to the coordinator in response to the measured input exceeding a predetermined threshold.
21. The sensor device of examples 1-20, wherein the coordinator is in communication with a central server, and wherein the coordinator is configured to transmit data from the at least one sensor to the central server.
22. The sensor device of example 21, wherein the coordinator is configured to filter the data from the at least one sensor into reduced data prior to transmission to the central server.
23. The sensor device of examples 21-22, wherein the data is transmitted to the central server in real-time.
24. The sensor device of examples 21-22, wherein the data is transmitted to the central server in batch format.
25. The sensor device of examples 1-20, wherein the sensor device is configured to filter the data into a reduced subset of data.
26. The sensor device of example 25, wherein the sensor device is configured to transmit the reduced subset of data to at least one of the coordinator or the central server.
27. The sensor device of example 26, wherein the reduced subset of data is transmitted to the at least one of the coordinator or the central server in real-time.
28. The sensor device of examples 1-20 and 25-27, wherein the sensor device is configured to transmit the data to a coordinator, wherein the coordinator is in communication with the central server.

### Example Set 7

1. A device comprising:
   a sensor;
   a transceiver;
   a processor in communication with the sensor and the transceiver; and
   a memory in communication with the processor and storing instructions executable by the processor for:
      receiving data from the sensor; and
      transmitting at least a portion of the data to another device via the transceiver.
2. The device of example 1, further comprising a power source for powering the device.
3. The device of example 2, wherein the power source comprises one or more of a battery and a capacitor.
4. The device of example 3, wherein the power source comprises a battery, and the device further comprises an energy harvester coupled to the power source for recharging the battery.
5. The device of example 4, wherein the energy harvester includes one or more of a photovoltaic cell for collecting solar energy; a thermoelectric generator (TEG); and/or a piezoelectric vibrational energy harvester (PZEH).
6. The device of examples 1-5, wherein the sensor comprises an accelerometer.
7. The device of example 6, wherein the sensor comprises an integrated, printed circuit board (PCB) mounted accelerometer sensor.
8. The device of examples 1-7, wherein the device is configured to be mounted directly to rotating machinery equipment selected from the group consisting of: a reciprocating engine and a compressor.
9. The device of examples 1-8, wherein the memory further stores instructions for comparing the data from the sensor against previously-collected data from the sensor.
10. The device of example 9, wherein the memory further stores instructions to determine, based on the comparison, whether to take an action.
11. The device of example 10, wherein the action to be taken includes one or more of: sending an alarm, reporting a condition, and disabling equipment being monitored.
12. The device of examples 1-11, wherein the memory further stores instructions to calculate mechanical revolutions per minute (RPM) of a crankshaft of an engine monitored by the sensor.
13. The device of any of examples 1-12, further comprising a plurality of sensors.
14. The device of example 12, wherein the memory further stores instructions for:
   gathering data related to the operation of an engine from a first sensor;
   gathering environmental data regarding the engine from a second sensor; and
   determining a nominal operating characteristic for the engine based on the data from the first sensor and the data from the second sensor.
15. The device of example 14, wherein the first sensor is configured to detect one or more of cylinder temperature, valve vibration, main bearing vibration, and combinations thereof
16. The device of examples 14-15, wherein the second sensor is configured to detect one or more of geographical location and meteorological information.
17. The device of examples 14-16, wherein the memory further stores instructions to detect a variation from the nominal operating characteristic and transmit an alert via the transceiver, the alert indicating the variation.

### Example Set 8

1. A device for monitoring the function of an engine, the device comprising:
   (a) a housing mounted to the engine.
   (b) a power source.
   (c) one or more sensors, each of which detect an engine condition. and
   (d) a transmitter for transmitting each of the detected engine conditions, the transmitter powered by the power source.
2. The device of example 1 that further includes a processor in communication with each of the one or more sensors, the processor for receiving data regarding each of the engine conditions and converting the data into electronic signals that are transmitted by the transmitter.
3. The device of example 1 that further includes a database for storing at least some of the detected engine conditions.
4. The device of example 3 wherein the database is part of the processor.
5. The device of example 2 wherein the processor is inside of the housing.
6. The device of example 3 wherein the processor is inside of the housing.
7. The device of example 1 wherein the power source is inside the housing.
8. The device of example 1 wherein the power source is a battery.
9. The device of example 8 wherein the battery is a LiPON battery.
10. The device of example 1 that further includes a secondary power source.
11. The device of example 10 wherein the secondary power source is a battery.
12. The device of example 10 wherein the secondary power source is inside of the housing.
13. The device of example 11 wherein the battery is a lithium thynol chloride battery.
14. The device of example 1 wherein the housing is a two-piece housing.
15. The device of example 14 wherein the two-piece housing comprises a top half and a bottom half.
16. The device of example 15 wherein the top half is comprised of a material that is more thermally conductive than the material comprising the bottom half.
17. The device of example 16 wherein the top half is comprised of metal and the bottom half is comprised of plastic.
18. The device of example 17 wherein the top half is comprised of injection-molded aluminum.
19. The device of example 17 wherein the bottom half is comprised of PPS.
20. The device of example 15 wherein the bottom half can withstand a temperature of at least 100° C. without losing its structural integrity.
21. The device of example 15 wherein the top half has thermally-conductive projections to dissipate heat.
22. The device of example 21 wherein the thermally-conductive projections are comprised of one or more of fins and rods.
23. The device of example 1 that is configured so that the temperature inside the housing does not exceed 85° C.
24. The device of example 1 wherein the housing includes posts for mounting it to the engine.
25. The device of example 24 wherein the posts are between ½″ and 1½″ in length.
26. The device of example 15 wherein the bottom half of the housing includes posts for mounting the housing to the engine.
27. The device of example 26 wherein the posts are between ½″ and 1½″ in length.
28. The device of example 27 wherein there are four posts.
29. The device of example 15 wherein a gasket is mounted between the top half and the bottom half.
30. The device of example 26 wherein each post includes a channel for receiving a bolt.
31. The device of example 26 wherein each post includes a channel and a screw boss inside of the channel, each screw boss for receiving a bolt.
32. The device of example 1 that further includes a system for recharging the power source.
33. The device of example 8 that further includes a system for recharging the power source.
34. The device of example 33 wherein the battery is inside the housing, and the system for recharging the battery includes a heat pipe.
35. The device of example 8 that includes a system for recharging the battery and the system for recharging the power source includes a heat pipe.
36. The device of example 35 wherein the heat pipe is comprised of metal.
37. The device of example 36 wherein the heat pipe is comprised of aluminum.
38. The device of example 33 wherein the system for recharging the battery includes:
   (a) a heat pipe that is at least partially contained within the housing, the heat pipe having a first end, a second end and a body portion.
   (b) a thermal energy generator adjacent the first end of the heat pipe for receiving heat from the heat pipe, the thermal energy generator for generating electrical power. and
   (c) the second end of the heat pipe adjacent a source of heat so as to transmit the heat through the body to the first end and to the thermal energy generator.
39. The device of example 34 that further includes an opening in the bottom half of the housing, the opening dimensioned to receive the body of the heat pipe.
40. The device of example 39 wherein the body of the heat pipe is positioned in the opening and the second end of the heat pipe is positioned outside of the housing.
41. The device of example 38 wherein there is a first flexible membrane between the first end of the heat pipe and the thermal energy generator.
42. The device of example 41 wherein the upper half of the housing has an inner surface and there is a second flexible membrane between the thermal energy generator and the inner surface of the upper half of the housing.
43. The device of example 41 wherein there is between 100 psi and 200 psi of pressure against the first flexible membrane to conform it to the surface of the thermal energy generator.
44. The device of example 38 that further includes a retainer at the first end of the heat pipe, the retainer for retaining the thermal energy generator.
45. The device of example 44 wherein the retainer is comprised of plastic.
46. The device of example 40 wherein the bottom half of the housing has an inner surface, and there is a gasket surrounding the opening, the gasket on the inner surface, the gasket for sealing between the heat pipe and the opening.
47. The device of example 38 wherein a portion of the body of the heat pipe adjacent the second end is surrounded by insulating material.
48. The device of example 47 wherein the insulating material is a plastic sleeve.
49. The device of example 38 that further includes a biasing element to bias the heat pipe towards the upper half of the housing.
50. The device of example 49 wherein the biasing element is a spring surrounding part of the body of the heat pipe.
51. The device of example 15 wherein the top portion of the housing has one or more openings, wherein each opening is configured to receive a coupling.
52. The device of example 15 wherein the top portion of the housing has one or more openings with a coupling in each opening, and each coupling is configured to be coupled to sensor.
53. The device of example 52 that further includes a processor, wherein each coupling is in electronic communication with the processor.
54. The device of example 38 wherein the thermal energy generator is in communication with the processor and sends electric power to the processor.
55. The device of example 54 wherein the processor is in communication with the first battery and transfers electric power from the thermal energy generator to the battery to recharge the battery.

### Example Set 9

1. A holding tank monitoring system comprising:
   a sensor device configured to receive total dissolved solids (TDS) data of a stored fluid from a TDS sensor in real-time, wherein the TDS sensor is located near an input of a holding tank storing the stored fluid;
   wherein the TDS sensor data is used to determine water production of a natural resource well; and
   wherein predictive analysis is used to determine expected remaining production of the well based in part on the water production.
2. The holding tank monitoring system of example 1, wherein the TDS sensor is an electrical conductivity meter.
3. The holding tank monitoring system of example 2, wherein the electrical conductivity meter is configured to measure a salt solution percentage of the stored fluid.
4. The holding tank monitoring system of examples 1-3, wherein the stored fluid is water by-product produced by a fracking well.
5. The holding tank monitoring system of examples 1-4, further comprising a central server configured to receive the TDS data from the sensor device.
6. The holding tank monitoring system of example 5, wherein the TDS data is transmitted to the central server in real-time.
7. The holding tank monitoring system of example 5, wherein the TDS data is transmitted to the central server in batch format.
8. The holding tank monitoring system of examples 1-4, wherein the sensor device is configured to filter the TDS data into a reduced subset of TDS data.
9. The holding tank monitoring system of example 8, wherein the sensor device is configured to transmit the reduced subset of TDS data to at least one of the coordinator or the central server.
10. The holding tank monitoring system of example 9, wherein the reduced subset of TDS data is transmitted to the at least one of the coordinator or the central server in real-time.
11. The holding tank monitoring system of examples 1-4, wherein the sensor device is configured to transmit the TDS data to a coordinator, wherein the coordinator is in communication with the central server.
12. The holding tank monitoring system of example 11, wherein the coordinator is configured to filter the TDS data into a reduced subset of TDS data.
13. The holding tank monitoring system of example 12, wherein the coordinator is configured to transmit the reduced subset of TDS data to the central server.
14. The holding tank monitoring system of example 13, wherein the reduced subset of TDS data is transmitted to the central server in real-time.
15. The holding tank monitoring system of example 13, wherein the reduced subset of TDS data is transmitted to the central server in batch format.
16. The holding tank monitoring system of examples 1-15, wherein the sensor device comprises:
   a controller operatively coupled to the TDS sensor, wherein the controller is configured to receive the TDS data from the TDS sensor; and
   a wireless communication device coupled to the controller, wherein the wireless communication device is configured to communicate with the central server.
17. The holding tank monitoring system of example 16, wherein the sensor device further comprises:
   a processor in communication with the TDS sensor and the wireless communication device; and
   a memory in communication with the processor and storing instructions executable by the processor for:
      receiving the TDS data from the TDS sensor; and
      transmitting at least a portion of the TDS data to another sensor device via the wireless communication device.
18. The sensor device of examples 1-17, further comprising a power source for powering the sensor device.
19. The sensor device of example 18, wherein the power source comprises one or more of a battery and a capacitor.
20. The sensor device of example 19, wherein the power source comprises a battery, and the sensor device further comprises an energy harvester coupled to the power source for recharging the battery.
21. The sensor device of example 20, wherein the energy harvester includes one or more of a photovoltaic cell for collecting solar energy; a thermoelectric generator (TEG); and/or a piezoelectric vibrational energy harvester (PZEH).
22. The holding tank monitoring system of examples 16-21, wherein the wireless communication device is configured to transmit using at least one of a satellite communication network, a local area network (LAN), a wide area network (WAN), a wireless mobile telephone network, a General Packet Radio Service (GPRS) network, a wireless local area network (WLAN), a Global System for Mobile Communications (GSM) network, a Personal Communication Service (PCS) network, and an Advanced Mobile Phone System (AMPS) network.
23. The holding tank monitoring system of examples 1-22, wherein the sensor device is one of a plurality of sensor devices in the holding tank monitoring system.
24. The holding tank monitoring system of examples 1-23, wherein the predictive analysis is additionally based on past water production data from the natural resource well.
25. A holding tank monitoring method comprising:
   receiving, by a sensor device, total dissolved solids (TDS) data of a stored fluid from a TDS sensor in real-time, wherein the TDS sensor is located near an input of a holding tank storing the stored fluid;
   determining water production of a natural resource well based on the TDS sensor data, and
   determining expected remaining production of the well using predictive analysis based in part on the water production.
26. The holding tank monitoring method of example 25, wherein the TDS data is transmitted to a central server in real-time.
27. The holding tank monitoring method of example 25, wherein the TDS data is transmitted to a central server in batch format.
28. The holding tank monitoring method of examples 25-27, wherein the natural resource well is a fracking well, and wherein the stored fluid is water by-product produced by the fracking well.
29. The holding tank monitoring method of examples 25-28, wherein the TDS sensor is an electrical conductivity meter.
30. The holding tank monitoring method of example 29, wherein the electrical conductivity meter is configured to measure a salt solution percentage of the stored fluid.
31. The holding tank monitoring method of examples 27-30, wherein the sensor device comprises:
   a controller operatively coupled to the TDS sensor, wherein the controller is configured to receive the TDS data from the TDS sensor; and
   a wireless communication device coupled to the controller, wherein the wireless communication device is configured to communicate with the central server.
32. The holding tank monitoring method of examples 25-33, wherein the predictive analysis is additionally based on past water production data from the natural resource well.

### Example Set 10

1. A logistics method comprising:
   receiving data from a plurality of sensor devices, wherein each of the plurality of sensor devices is in communication with an individual holding tank, and wherein the data comprises a flow rate of the individual holding tanks, and wherein the data identifies the individual holding tank locations;
   determining a remaining time period until each of the individual holding tanks reaches capacity based on the flow rate and a remaining capacity of the individual holding tanks;
   identifying a fleet of tanker trucks for draining the individual holding tanks; and
   using the data to populate a mathematical model that comprises an objective function for minimizing tanker truck driven miles and preventing the individual holding tanks from reaching capacity.
2. The logistics method of example 1, wherein the data is provided to a sensor device by a flow meter coupled to the individual holding tank.
3. The logistics method of examples 1-2, wherein the data is provided to a sensor device by a pressure transducer coupled to the individual holding tank.
4. The logistics method of examples 1-3, further comprising determining a prioritized order of draining the tanks in the system based in part on the remaining time period of the individual holding tank.
5. The logistics method of examples 1-4, wherein each of the plurality of sensor devices comprises:
   at least one sensor operatively coupled to a controller, wherein the controller is configured to receive a measured input from the at least one sensor; and
   a wireless communication device coupled to the controller, wherein the wireless communication device is configured to communicate with a central server.
6. The logistics method of example 5, wherein the wireless communication device is configured to transmit using at least one of a satellite communication network, a local area network (LAN), a wide area network (WAN), a wireless mobile telephone network, a General Packet Radio Service (GPRS) network, a wireless local area network (WLAN), a Global System for Mobile Communications (GSM) network, a Personal Communication Service (PCS) network, and an Advanced Mobile Phone System (AMPS) network.
7. The logistics method of examples 5-6, wherein the at least one sensor is at least one of a flow meter and a pressure transducer.
8. A logistics system comprising:
   a plurality of sensor devices providing data, wherein each of the plurality of sensor devices is in communication with an individual holding tank, and wherein the data comprises flow rate of the individual holding tanks, and wherein the data identifies the individual holding tank locations;
   a capacity module configured to determine the time remaining until each of the individual holding tanks reaches capacity based on the flow rate and remaining capacity of the individual holding tanks;
   an identification module configured to identify a fleet of tanker trucks for draining the individual holding tanks; and
   a processor implementing a mathematical model populated by the data, wherein the mathematical model comprises an objective function for minimizing tanker truck driven miles and preventing the individual holding tanks from reaching capacity.
9. The logistics system of example 8, wherein the data is provided to a sensor device by a flow meter coupled to the individual holding tank.
10. The logistics system of examples 8-9, wherein the data is provided to a sensor device by a pressure transducer coupled to the individual holding tank.
11. The logistics system of examples 8-10, wherein the order of draining the tanks in the system is determined in part by whether a first individual holding tank is closer to overflowing than a second holding tank.
12. The logistics system of examples 8-11, wherein each of the plurality of sensor devices comprises:
   at least one sensor operatively coupled to a controller, wherein the controller is configured to receive a measured input from the at least one sensor; and
   a wireless communication device coupled to the controller, wherein the wireless communication device is configured to communicate with a central server.
13. The logistics system of example 12, wherein the sensor device further comprise:
   a processor in communication with the sensor and the wireless communication device; and
   a memory in communication with the processor and storing instructions executable by the processor for:
      receiving data from the sensor; and
      transmitting at least a portion of the data to another sensor device via the wireless communication device.
14. The logistics system of example 13, further comprising a power source for powering the sensor device.
15. The logistics system of example 14, wherein the power source comprises one or more of a battery and a capacitor.
16. The logistics system of example 15, wherein the power source comprises a battery, and the sensor device further comprises an energy harvester coupled to the power source for recharging the battery.
17. The logistics system of example 16, wherein the energy harvester includes one or more of a photovoltaic cell for collecting solar energy; a thermoelectric generator (TEG); and/or a piezoelectric vibrational energy harvester (PZEH).
18. The logistics system of examples 12-17, wherein the wireless communication device is configured to transmit using at least one of a satellite communication network, a local area network (LAN), a wide area network (WAN), a wireless mobile telephone network, a General Packet Radio Service (GPRS) network, a wireless local area network (WLAN), a Global System for Mobile Communications (GSM) network, a Personal Communication Service (PCS) network, and an Advanced Mobile Phone System (AMPS) network.
19. The logistics system of example 12, wherein the at least one sensor is at least one of a flow meter and a pressure transducer.
20. The logistics system of examples 8-19, wherein the sensor device is configured to filter the data into a reduced subset of data.
21. The logistics system of example 20, wherein the sensor device is configured to transmit the reduced subset of data to at least one of the coordinator or the central server.
22. The logistics system of example 21, wherein the reduced subset of data is transmitted to the at least one of the coordinator or the central server in real-time.
23. The logistics system of examples 8-19, wherein the sensor device is configured to transmit the data to a coordinator, wherein the coordinator is in communication with the central server.
24. The logistics system of example 23, wherein the coordinator is configured to filter the data into a reduced subset of data.
25. The logistics system of example 24, wherein the coordinator is configured to transmit the reduced subset of data to the central server.
26. The logistics system of example 25, wherein the reduced subset of data is transmitted to the central server in real-time.
27. The logistics system of example 25, wherein the reduced subset of data is transmitted to the central server in batch format.

### Example Set 11

1. A volatile organic compound (VOC) sensor device comprising:
   a sensor located in proximity to a tank vent of a storage tank, wherein the sensor is configured to monitor flumes from the tank vent;
   a controller operatively coupled to the sensor, wherein the controller is configured to receive a measured input from the sensor, wherein the measured input is VOC measurement data of the flumes; and
   a wireless communication device coupled to the controller, wherein the wireless communication device is configured to communicate with a coordinator.
2. The VOC sensor device of example 1, further comprising:
   a processor in communication with the sensor and the wireless communication device; and
   a memory in communication with the processor and storing instructions executable by the processor for:
      receiving data from the sensor; and
      transmitting at least a portion of the data to another sensor device via the wireless communication device.
3. The VOC sensor device of examples 1-2, further comprising a power source for powering the VOC sensor device.
4. The VOC sensor device of example 3, wherein the power source comprises one or more of a battery and a capacitor.
5. The VOC sensor device of example 4, wherein the power source comprises a battery, and the VOC sensor device further comprises an energy harvester coupled to the power source for recharging the battery.
6. The VOC sensor device of example 5, wherein the energy harvester includes one or more of a photovoltaic cell for collecting solar energy; a thermoelectric generator (TEG); and/or a piezoelectric vibrational energy harvester (PZEH).
7. The VOC sensor device of examples 1-6, wherein the sensor is an infrared thermal monitor.
8. The VOC sensor device of examples 1-7, wherein the VOC measurement data measures levels of benzene, toluene, ethylbenzene, and xylenes.
9. The VOC sensor device of examples 1-8, wherein the VOC measurement data is used to calculate fugitive losses from the tank vent.
10. The VOC sensor device of example 9, wherein the coordinator is in communication with a central server.
11. The VOC sensor device of example 10, wherein at least one of the coordinator and the central server is configured to analyze the VOC measurement data to determine if regulations are satisfied.
12. The VOC sensor device of example 11, wherein the regulations are set by a government agency.
13. The VOC sensor device of examples 1-12, wherein the wireless communication device is configured to transmit using at least one of a satellite communication network, a local area network (LAN), a wide area network (WAN), a wireless mobile telephone network, a General Packet Radio Service (GPRS) network, a wireless local area network (WLAN), a Global System for Mobile Communications (GSM) network, a Personal Communication Service (PCS) network, and an Advanced Mobile Phone System (AMPS) network.
14. The VOC sensor device of examples 1-13, wherein the VOC sensor device is configured to filter the data into a reduced subset of data.
15. The VOC sensor device of example 14, wherein the VOC sensor device is configured to transmit the reduced subset of data to at least one of the coordinator or the central server.
16. The VOC sensor device of example 15, wherein the reduced subset of data is transmitted to the at least one of the coordinator or the central server in real-time.
17. The VOC sensor device of examples 17-26, wherein the VOC sensor device is configured to transmit the data to a coordinator, wherein the coordinator is in communication with the central server.
18. The VOC sensor device of examples 1-13, wherein the coordinator is configured to filter the data into a reduced subset of data.
19. The VOC sensor device of example 18, wherein the coordinator is configured to transmit the reduced subset of data to a central server.
20. The VOC sensor device of example 19, wherein the reduced subset of data is transmitted to the central server in real-time.
21. The VOC sensor device of example 19, wherein the reduced subset of data is transmitted to the central server in batch format.
22. The VOC sensor device of examples 1-21, wherein the VOC sensor device is one of a plurality of sensor devices in a monitoring system.
23. A method of volatile organic compound (VOC) monitoring comprising:
monitoring, by a sensor located in proximity to a tank vent of a storage tank, flumes from the tank vent;
receiving, by a controller operatively coupled to the sensor, a measured input from the sensor, wherein the measured input is VOC measurement data of the flumes;
   communicating, by a wireless communication device coupled to the controller, with a coordinator.
24. The method of example 23, wherein the sensor is an infrared thermal monitor.
25. The method of examples 23-24, wherein the VOC measurement data measures levels of benzene, toluene, ethylbenzene, and xylenes.
26. The method of examples 23-25, further comprising calculating fugitive losses from the tank vent based on the VOC measurement data.
27. The method of example 26, wherein the coordinator is in communication with a central server.
28. The method of example 27, further comprising analyzing, by at least one of the coordinator and the central server, the VOC measurement data to determine if regulations are satisfied.
29. The method of example 28, wherein the regulations are set by a government agency.
30. The method of examples 23-29, wherein the wireless communication device is configured to transmit using at least one of a satellite communication network, a local area network (LAN), a wide area network (WAN), a wireless mobile telephone network, a General Packet Radio Service (GPRS) network, a wireless local area network (WLAN), a Global System for Mobile Communications (GSM) network, a Personal Communication Service (PCS) network, and an Advanced Mobile Phone System (AMPS) network.
31. The method of examples 23-30, wherein the sensor device is one of a plurality of sensor devices in a monitoring system.

### Example Set 12

1. A method of selective holding tank draining comprising:
   receiving, by a sensor device, total dissolved solids (TDS) data of a stored fluid from a TDS sensor;
   receiving, by the sensor device, volume data of the stored fluid from a volume sensor;
   determining, by a central server, a selected TDS level for disposal of the stored fluid;
   calculating an average TDS level of a drained volume of the stored fluid if draining from two or more tanks; and
   determining a stored fluid volume to drain from each of the two or more tanks to achieve a drained mixture have less than the selected TDS level.
2. The method of example 1, wherein the TDS sensor is coupled to the holding tank near an input of the stored fluid.
3. The method of examples 1-2, wherein the TDS sensor is an electrical conductivity meter.
4. The method of example 3, wherein the electrical conductivity meter is configured to measure a salt solution percentage of the stored fluid.
5. The method of examples 1-4, wherein the volume sensor is a pressure transducer.
6. The method of example 5, wherein the pressure transducer is located near the bottom of the holding tank.
7. The method of examples 1-6, wherein the volume of the drained mixture is less than the capacity of a tanker truck.
8. The method of examples 1-7, wherein the sensor device comprises:
   a controller operatively coupled to the TDS sensor, wherein the controller is configured to receive the TDS data from the TDS sensor; and
   a wireless communication device coupled to the controller, wherein the wireless communication device is configured to communicate with the central server.
9. The method of example 8, wherein the wireless communication device is configured to transmit using at least one of a satellite communication network, a local area network (LAN), a wide area network (WAN), a wireless mobile telephone network, a General Packet Radio Service (GPRS) network, a wireless local area network (WLAN), a Global System for Mobile Communications (GSM) network, a Personal Communication Service (PCS) network, and an Advanced Mobile Phone System (AMPS) network.
10. The method of examples 1-9, wherein the selected TDS level is one of a plurality of TDS levels, wherein the disposal requirements of the drained mixture is determined by regulations corresponding to the plurality of TDS levels.
11. The method of example 10, wherein the regulations are set by a government agency.
12. The method of examples 1-11, wherein the TDS data and the volume data are transmitted to the central server in real-time.
13. The method of examples 1-11, wherein the TDS data and the volume data are transmitted to the central server in batch-format.
14. The method of examples 1-13, further comprising transmitting, by the sensor device, the TDS data and the volume data to a coordinator, wherein the coordinator is in communication with the central server.
15. The method of example 14, further comprising filtering, by the coordinator, the TDS data and volume data into a reduced subset of TDS and volume data.
16. The method of example 15, further comprising transmitting, by the coordinator, the reduced subset of TDS and volume data to the central server.
17. The method of example 16, wherein the reduced subset of TDS and volume data is transmitted to the central server in real-time.
18. The method of example 16, wherein the reduced subset of TDS and volume data is transmitted to the central server in batch format.
19. The method of examples 16-18, wherein the reduced subset of TDS and volume data is transmitted to the central server during off-peak times.
20. The method of examples 1-22, wherein the selected TDS level is one of a plurality of TDS levels, wherein the disposal requirements of the drained mixture is determined by regulations corresponding to the plurality of TDS levels.
21. The method of example 20, wherein the regulations are set by a government agency.
22. A selective holding tank draining system comprising:
   a sensor device configured to receive total dissolved solids (TDS) data of a stored fluid from a TDS sensor, and wherein the sensor device is configured to receive volume data of the stored fluid from a volume sensor;
   a central server configured to determine a selected TDS level for disposal of the stored fluid;
   wherein an average TDS level of a drained volume of the stored fluid if draining from two or more tanks is calculated; and
   wherein a stored fluid volume to drain from each of the two or more tanks to achieve a drained mixture have less than the selected TDS level is determined.
23. The selective holding tank draining system of example 22, wherein the TDS sensor is an electrical conductivity meter.
24. The selective holding tank draining system of example 23, wherein the electrical conductivity meter is configured to measure a salt solution percentage of the stored fluid.
25. The selective holding tank draining system of examples 22-24, wherein the volume sensor is a pressure transducer.
26. The selective holding tank draining system of examples 22-25, wherein the volume of the drained mixture is less than the capacity of a tanker truck.
27. The selective holding tank draining system of examples 22-26, wherein the sensor device comprises:
   a controller operatively coupled to the TDS sensor and the volume sensor, wherein the controller is configured to receive the TDS data from the TDS sensor and receive the volume data from the volume sensor; and
   a wireless communication device coupled to the controller, wherein the wireless communication device is configured to communicate with the central server.
28. The selective holding tank draining system of example 27, wherein the sensor device further comprises:
   a processor in communication with the TDS sensor, the volume sensor and the wireless communication device; and
   a memory in communication with the processor and storing instructions executable by the processor for:
      receiving data from the TDS sensor and the volume sensor; and
      transmitting at least a portion of the TDS data and the volume data to another sensor device via the wireless communication device.
29. The selective holding tank draining system of examples 22-28, further comprising a power source for powering the sensor device.
30. The selective holding tank draining system of example 29, wherein the power source comprises one or more of a battery and a capacitor.
31. The selective holding tank draining system of example 30, wherein the power source comprises a battery, and the sensor device further comprises an energy harvester coupled to the power source for recharging the battery.
32. The selective holding tank draining system of example 31, wherein the energy harvester includes one or more of a photovoltaic cell for collecting solar energy; a thermoelectric generator (TEG); and/or a piezoelectric vibrational energy harvester (PZEH).
33. The selective holding tank draining system of examples 22-32, wherein the selected TDS level is one of a plurality of TDS levels, wherein the disposal requirements of the drained mixture is determined by regulations corresponding to the plurality of TDS levels.
34. The selective holding tank draining system of example 33, wherein the regulations are set by a government agency.
35. The selective holding tank draining system of examples 22-34, wherein the TDS data and the volume data are transmitted to the central server in real-time.
36. The selective holding tank draining system of examples 22-34, wherein the TDS data and the volume data are transmitted to the central server in batch-format.
37. The selective holding tank draining system of example 22-34, wherein the sensor device is configured to filter the TDS data and the volume data into a reduced subset of TDS data and volume data.
38. The selective holding tank draining system of example 37, wherein the sensor device is configured to transmit the reduced subset of TDS data and volume data to at least one of the coordinator or the central server.
39. The selective holding tank draining system of example 38, wherein the reduced subset of TDS data and volume data is transmitted to the at least one of the coordinator or the central server in real-time.
40. The selective holding tank draining system of examples 22-34, wherein the sensor device is configured to transmit the TDS data and the volume data to a coordinator, wherein the coordinator is in communication with the central server.
41. The selective holding tank draining system of example 40, wherein the coordinator is configured to filter the TDS data and volume data into a reduced subset of TDS and volume data.
42. The selective holding tank draining system of example 41, wherein the coordinator is configured to transmit the reduced subset of TDS and volume data to the central server.
43. The selective holding tank draining system of example 42, wherein the reduced subset of TDS and volume data is transmitted to the central server in real-time.
44. The selective holding tank draining system of example 42, wherein the reduced subset of TDS and volume data is transmitted to the central server in batch format.
45. A tank for storing liquids, the tank including an entrance through which liquid can enter and an exit through which liquid can exit; the tank further having a volume and a device to measure one or more of (a) the volume of liquid in the tank, (b) the composition of any water in the tank, (c) the amount of total dissolve solids (TSD) in the tank, (d) the amount of any crude oil in the tank, and (e) the chemical composition of any crude oil in the tank.
46. The tank of example 45 wherein fluid enters the entrance directly or indirectly from an oil well.
47. The tank of example 45 wherein fluid is removed through the exit and placed into a tanker truck.
48. The tank of any of examples 45-47 wherein the device is self-powered.
49. The tank of example 48 wherein the device includes a secondary battery.
50. The tank of example 48 wherein the device includes a primary battery.
51. The tank of any of examples 45-50 wherein the device is powered by one or more of a solar collector, piezo chip, and a thermal energy source.
52. The tank of example 49 wherein the secondary battery is recharged by one or more of a solar collector, piezo chip, and a thermal energy source.
53. The tank of any of examples 45-52 wherein the device measures the volume of liquid in the tank.
54. The tank of any of examples 45-53 wherein the device measures the volumetric rate of liquid entering the tank over time.
55. The tank of any of examples 45-54 wherein the device measures the chemical composition of liquid in the tank.
56. The tank of any of examples 45-55 wherein the device measures the TDS of the liquid in the tank.
57. The tank of any of examples 45-56 wherein the device measures the salinity of at least some liquid in the tank.
58. The tank of any of examples 45-57 wherein the device measures the amount of crude oil in the tank.
59. The tank of any of examples 45-58 wherein the device measures the percentage of crude oil within the tank.
60. The tank of example 59 wherein the device measures the amount of sulfur in the crude oil within the tank.
61. The tank of any of examples 45-60 wherein the device includes a memory for storing information about the fluid collected in a tank.
62. The tank of any of examples 45-61 wherein the device includes a transmitter for transmitting some or all of the data it has collected.
63. A method for scheduling the draining of a storage tank having a predetermined volume, the method comprising:
   (a) measuring the volume of fluid in the storage tank;
   (b) measuring the flow rate of liquid into the tank; and
   (c) scheduling a truck to drain the storage tank based on its predetermined volume, the volume of liquid inside the tank, and the rate upon which fluid is entering it.
64. The method of example 63 wherein a truck is scheduled to empty the tank based on the chemical composition of the fluid inside the tank.
65. A method for scheduling the drainage of a storage tank having a predetermined volume, the scheduling time for drainage based upon one or more of the following parameters:
   (a) the volume of liquid in the tank,
   (b) the composition of any water in the tank,
   (c) the amount of total dissolve solids (TSD) in the tank,
   (d) the amount of any crude oil in the tank, and
   (e) the chemical composition of any crude oil in the tank.
66. The method of example 65 wherein a truck is scheduled to empty the tank.
67. The method of example 66 wherein the truck is emptied at a location based upon the chemical composition of the fluid inside the tank.
68. The method of any of examples 65-67 wherein a first set of one or more trucks is used to empty liquid from a first set of one or more tanks having liquid within a first range of parameters, and a second set of one or more trucks is used to empty liquid from a second set of one or more tanks having liquid within a second set of parameters.

### Example Set 13

1. An air monitoring array system comprising:
   a plurality of air quality sensor devices arranged within a selected area, wherein the plurality of air quality sensor devices is configured to measure air pollutant levels in the selected area;
   wherein each of the plurality of air quality sensor devices comprise:
      at least one sensor operatively coupled to a controller, wherein the controller is configured to receive a measured input from the at least one sensor; and
      a wireless communication device coupled to the controller, wherein the wireless communication device is configured to communicate with a central server.
2. The air monitoring array system of example 1, wherein the central server is configured to determine if one or more portions of the selected area have air pollutant levels exceeding a predetermined threshold.
3. The air monitoring array system of examples 1-2, wherein the predetermined threshold is set by a government agency.
4. The air monitoring array system of examples 1-3, wherein the at least one sensor is an e-nose sensor circuit.
5. The air monitoring array system of examples 1-3, wherein the at least one sensor is a hydrocarbon sensor.
6. The air monitoring array system of examples 1-5, wherein the at least one sensor is configured to monitor benzene levels.
7. The air monitoring array system of examples 1-6, wherein each of the plurality of air quality sensor devices is powered by solar power.
8. The air monitoring array system of examples 1-7, wherein each of the plurality of air quality sensor devices is powered by a battery.
9. The air monitoring array system of examples 1-8, further comprising a plurality of coordinators, wherein each of the plurality of coordinators is in communication with one or more sensor devices of the plurality of air quality sensor devices.
10. The air monitoring array system of examples 1-9, wherein each of the plurality of air quality sensor devices further comprises a temperature sensor for determining the ambient temperature at the at least one sensor.
11. The air monitoring array system of examples 1-10, wherein each of the plurality of air quality sensor devices further comprises an ultraviolet sensor for measuring ultraviolet levels at the at least one sensor.
12. The air monitoring array system of examples 1-11, wherein each of the plurality of air quality sensor devices further comprises an anemometer for measuring wind speed at the at least one sensor.
13. The air monitoring array system of examples 1-12, wherein the wireless communication device is a Yagi antenna.
14. The air monitoring array system of examples 1-6 and 9-13, wherein the sensor device further comprises:
   a processor in communication with the at least one sensor and the wireless communication device; and
   a memory in communication with the processor and storing instructions executable by the processor for:
      receiving data from the at least one sensor; and
      transmitting at least a portion of the data to another sensor device via the wireless communication device.
15. The air monitoring array system of example 13, further comprising a power source for powering the sensor device.
16. The air monitoring array system of example 15, wherein the power source comprises one or more of a battery and a capacitor.
17. The air monitoring array system of example 16, wherein the power source comprises a battery, and the sensor device further comprises an energy harvester coupled to the power source for recharging the battery.
18. The air monitoring array system of example 17, wherein the energy harvester includes one or more of a photovoltaic cell for collecting solar energy; a thermoelectric generator (TEG); and/or a piezoelectric vibrational energy harvester (PZEH).
19. The air monitoring array system of examples 1-18, wherein the sensor device is configured to filter the data into a reduced subset of data.
20. The air monitoring array system of examples 1-19, wherein the sensor device is configured to transmit the reduced subset of data to at least one of the coordinator or the central server.
21. The air monitoring array system of example 20, wherein the reduced subset of data is transmitted to the at least one of the coordinator or the central server in real-time.
22. The air monitoring array system of examples 1-18, wherein the sensor device is configured to transmit the data to a coordinator, wherein the coordinator is in communication with the central server.
23. The air monitoring array system of example 22, wherein the coordinator is configured to filter the data into a reduced subset of data.
24. The air monitoring array system of example 23, wherein the coordinator is configured to transmit the reduced subset of data to the central server.
25. The air monitoring array system of example 24, wherein the reduced subset of data is transmitted to the central server in real-time.
26. The air monitoring array system of example 24, wherein the reduced subset of data is transmitted to the central server in batch format.
27. A method of air quality monitoring comprising:
   measuring, by a plurality of air quality sensor devices arranged within a selected area, air pollutant levels in the selected area;
   wherein each of the plurality of air quality sensor devices comprise:
      at least one sensor operatively coupled to a controller, wherein the controller is configured to receive a measured input from the at least one sensor; and
      a wireless communication device coupled to the controller, wherein the wireless communication device is configured to communicate with a central server.
28. The method of example 27, further comprising determining, by the central server, if one or more portions of the selected area have air pollutant levels exceeding a predetermined threshold.
29. The method of example 28, wherein the predetermined threshold is set by a government agency.
30. The method of examples 27-29, wherein the at least one sensor is an e-nose sensor circuit.
31. The method of examples 27-29, wherein the at least one sensor is a hydrocarbon sensor.
32. The method of examples 27-31, wherein the at least one sensor is configured to monitor benzene levels.
33. The method of examples 27-32, wherein each of the plurality of air quality sensor devices is powered by solar power.
34. The method of examples 27-33, wherein each of the plurality of air quality sensor devices is powered by a battery.
35. The method of examples 27-34, further comprising communicating, by each of a plurality of coordinators, with one or more sensor devices of the plurality of air quality sensor devices.
36. The method of examples 27-35, further comprising determining, by a temperature sensor of each of the plurality of air quality sensor devices, the ambient temperature at the at least one sensor.
37. The method of examples 27-36, further comprising measuring, by an ultraviolet sensor of each of the plurality of air quality sensor devices, ultraviolet levels at the at least one sensor.
38. The method of examples 27-37, further comprising measuring, by an anemometer of each of the plurality of air quality sensor devices, wind speed at the at least one sensor.
39. The method of examples 27-38, wherein the wireless communication device is a Yagi antenna.
40. The method of examples 27-39, wherein the wireless communication device is configured to transmit using at least one of a satellite communication network, a local area network (LAN), a wide area network (WAN), a wireless mobile telephone network, a General Packet Radio Service (GPRS) network, a wireless local area network (WLAN), a Global System for Mobile Communications (GSM) network, a Personal Communication Service (PCS) network, and an Advanced Mobile Phone System (AMPS) network.
41. The method of examples 27-40, further comprising determining a source of air pollutants based on air pollutant levels as measured by the plurality of air quality sensor devices.

### Example Set 14

1. A quality monitoring method comprising:
   receiving, by a sensor device, total dissolved solids (TDS) data of a stored fluid from a TDS sensor in real-time;
   transmitting, by the sensor device, the TDS data to a coordinator; and
   comparing the TDS data to a TDS threshold level.
2. The quality monitoring method of example 1, wherein the TDS data is transmitted to the coordinator in real-time.
3. The quality monitoring method of examples 1-2, further comprising transmitting, by the coordinator, the TDS data to a central server.
4. The quality monitoring method of example 3, wherein the TDS data is transmitted to the central server in real-time.
5. The quality monitoring method of example 3, wherein the TDS data is transmitted to the central server in batch format.
6. The quality monitoring method of examples 1-2, further comprising filtering, by the coordinator, the TDS data into reduced TDS data.
7. The quality monitoring method of example 6, further comprising transmitting, by the coordinator, the reduced TDS data to a central server.
8. The quality monitoring method of example 7, wherein the reduced TDS data is transmitted to the central server in real-time.
9. The quality monitoring method of example 7, wherein the reduced TDS data is transmitted to the central server in batch format.
10. The quality monitoring method of examples 1-2, further comprising filtering, by the sensor device, the TDS data into reduced TDS data.
11. The quality monitoring method system of example 10, further comprising transmitting, by the coordinator, the reduced TDS data to at least one of the coordinator or the central server.
12. The quality monitoring method of example 11, wherein the reduced TDS data is transmitted to the at least one of the coordinator or the central server in real-time.
13. The quality monitoring method of examples 10-12, wherein the sensor device is configured to transmit the reduced TDS data to a coordinator, wherein the coordinator is in communication with the central server.
14. The quality monitoring method of examples 1-13, wherein the stored fluid is water by-product produced by a fracking well.
15. The quality monitoring method of examples 1-14, further comprising notifying, by the sensor device, a central server in response to the TDS data exceeding the TDS threshold level.
16. The quality monitoring method of examples 1-15, wherein the TDS sensor is an electrical conductivity meter.
17. The quality monitoring method of example 16, wherein the electrical conductivity meter is configured to measure a salt solution percentage of the stored fluid.
18. A quality monitoring system comprising:
   a sensor device configured to receive total dissolved solids (TDS) data of a stored fluid from a TDS sensor in real-time; and
   a coordinator configured to receive the TDS data from the sensor device;
   wherein the TDS data is compared to a TDS threshold level.
19. The quality monitoring system of example 18, wherein the TDS data is transmitted to the coordinator in real-time.
20. The quality monitoring system of examples 18-19, wherein the coordinator transmits the TDS data to a central server.
21. The quality monitoring system of example 20, wherein the TDS data is transmitted to the central server in real-time.
22. The quality monitoring system of example 20, wherein the TDS data is transmitted to the central server in batch format.
23. The quality monitoring system of examples 18-19, wherein the coordinator is configured to filter the TDS data into reduced TDS data.
24. The quality monitoring system of example 23, wherein the coordinator transmits the reduced TDS data to a central server.
25. The quality monitoring system of example 24, wherein the reduced TDS data is transmitted to the central server in real-time.
26. The quality monitoring system of example 24, wherein the reduced TDS data is transmitted to the central server in batch format.
27. The quality monitoring system of example 18, wherein the sensor device is configured to filter the data into reduced TDS data.
28. The quality monitoring system of example 27, wherein the sensor device is configured to transmit the reduced TDS data to at least one of the coordinator or the central server.
29. The quality monitoring system of example 28, wherein the reduced TDS data is transmitted to the at least one of the coordinator or the central server in real-time.
30. The quality monitoring system of examples 27-29, wherein the sensor device is configured to transmit the reduced TDS data to a coordinator, wherein the coordinator is in communication with the central server.
31. The quality monitoring system of examples 18-30, wherein the stored fluid is water by-product produced by a fracking well.
32. The quality monitoring system of examples 18-31, wherein the sensor device notifies the central server in response to the TDS data exceeding the TDS threshold level.
33. The quality monitoring system of examples 18-32, wherein the TDS sensor is an electrical conductivity meter.
34. The quality monitoring system of example 33, wherein the electrical conductivity meter is configured to measure a salt solution percentage of the stored fluid.
35. The quality monitoring system of examples 18-34, wherein the sensor device comprises:
   a controller operatively coupled to the TDS sensor, wherein the controller is configured to receive the TDS data from the TDS sensor; and
   a wireless communication device coupled to the controller, wherein the wireless communication device is configured to communicate with the central server.
36. The quality monitoring system of example 35, wherein the sensor device further comprises:
   a processor in communication with the TDS sensor and the wireless communication device; and
   a memory in communication with the processor and storing instructions executable by the processor for:
      receiving the TDS data from the TDS sensor; and
      transmitting at least a portion of the TDS data to another sensor device via the wireless communication device.
37. The quality monitoring system of examples 18-36, further comprising a power source for powering the sensor device.
38. The quality monitoring system of example 37, wherein the power source comprises one or more of a battery and a capacitor.
39. The quality monitoring system of example 38, wherein the power source comprises a battery, and the sensor device further comprises an energy harvester coupled to the power source for recharging the battery.
40. The quality monitoring system of example 39, wherein the energy harvester includes one or more of a photovoltaic cell for collecting solar energy; a thermoelectric generator (TEG); and/or a piezoelectric vibrational energy harvester (PZEH).
41. The quality monitoring system of example 35-40, wherein the wireless communication device is configured to transmit using at least one of a satellite communication network, a local area network (LAN), a wide area network (WAN), a wireless mobile telephone network, a General Packet Radio Service (GPRS) network, a wireless local area network (WLAN), a Global System for Mobile Communications (GSM) network, a Personal Communication Service (PCS) network, and an Advanced Mobile Phone System (AMPS) network.
42. The quality monitoring system of examples 18-41, wherein the sensor device is one of a plurality of sensor devices in a monitoring system.

### Example Set 15

1. A device for transferring heat from a heat source to a thermal energy generator, the device including:
   (a) a heat pipe having a first end, a second end and body portion therebetween, the first end configured to be in contact with a heat source; and
   (b) at least one insulating sleeve surrounding at least part of the heat pipe, the insulating sleeve for reducing the escape of heat from the heat pipe to areas near the heat pump.
2. A valve cover for use with an engine, the valve cover including:
   (a) a plurality of first openings for receiving fasteners in order to fasten a device to the valve cover, each of the plurality of first openings having a first diameter;
   (b) a second opening dimensioned to receive an end of a heat pipe, so that the end of the heat pipe passes through the valve cover and is retained in the valve casing where it does not contact the valves.
3. The valve cover of example 2 that is comprised of metal.
4. The valve cover of example 2 wherein each of the plurality of first openings is configured to receive a threaded fastener.
5. A casing for an engine monitoring device, the casing comprising:
   (a) a first part that includes first openings for fasteners to attach the casing to a structure, and a second opening larger than any of the second openings, the second opening for permitting a heat pipe to pass therethrough; and
   (b) a second part that includes heat-transfer projections, the second part attachable to the first part.
6. The casing of example 5 that further includes a gasket that is positioned between the first part and the second part when the first part and second part are attached.
7. The casing of example 6 wherein the gasket is comprised of rubber.
8. The casing of example 5 wherein the heat-transfer projections on the second part comprise one or more of fins and rods.
9. The casing of example 8 wherein the second part has a main surface and at least some of the fins and rods extend outward at least ½″ from the main surface.
10. The casing of example 8 wherein each of the fins and rods are spaced apart between 1/32″ and ½″ from each of the other fins and rods.
11. The casing of example 5 wherein the first part is comprised of a thermally insulating material and the second part is comprised of a thermally conductive material.
12. The casing of example 11 wherein the first part is comprised of plastic and the second part is comprised of metal.
13. The casing of example 12 wherein the plastic is PTBE.
14. The casing of example 11 wherein the metal is aluminum.
15. The casing of example 5 wherein there is an opening in the first part.
16. The casing of example 5 wherein the first part has a bottom and that further includes a plurality of legs extending from the bottom.
17. The casing of example 16 wherein there are three or more legs.
18. The casing of example 16 wherein each leg has an opening for receiving a fastener.

Having thus described exemplary embodiments of the invention, other variations and embodiments that do not depart from the spirit of the invention will become apparent to those skilled in the art. The scope of the present invention is thus not limited to any particular embodiment, but is instead set forth in the appended claims and legal equivalents thereof. Unless expressly stated in the written description or the claims, the steps of any method recited in the claims can be performed in any order capable of yielding the desired result.

### Example Set 16

1. A pipe used for drilling, the pipe including a device mounted thereon, the device for measuring the vibration to which the pipe has been exposed.
2. The pipe of example 1 that includes a recess and the device is positioned in the recess.
3. The pipe of example 1 or example 2 wherein the device includes an accelerometer to measure vibration and a power source for powering the accelerometer.
4. The pipe of example 3 wherein the power source is a piezo chip.
5. The pipe of any of examples 1-4 that includes a memory for storing the vibrational data.
6. The pipe of any of examples 1-5 wherein the pipe has a first end with a first cross-sectional area and a second end having a second cross-sectional area, the second cross-sectional area being smaller than the first cross-sectional area.
7. The pipe of example 6 wherein the device is positioned on the second cross-sectional area.
8. The pipe of example 7 that includes a recess wherein the device is in the recess.
9. The pipe of example 8 wherein the recess is in the second end.
10. The pipe of example 8 or example 9 wherein the recess is between ⅛″ and 5/16″ deep.
11. The pipe of any of examples 1-10 wherein the device records the number of rotations of the pipe.
12. The pipe of any of examples 1-11 wherein the device records the vibration due to the material through the pipe is drilled.
13. The pipe of any of examples 1-12 wherein the device has a predetermined vibration quantity equal to the operational life of the pipe.
14. The pipe of example 13 wherein the measured vibration can be compared to the operational life to calculate the remaining life of the pipe.
15. The pipe of any of examples 1-14 wherein information from the device can be wirelessly extracted via a radio frequency signal.
16. A method of determining the operational life of a pipe, the method comprising the steps of:
   (a) attaching a device to the pipe, the device capable of measuring vibration applied to the pipe; and
   (b) operating the pipe, wherein vibration applied to the pipe is measured by a device.
17. The method of example 16 wherein the pipe is rotated and the vibration due to rotation is measured to determine the number of pipe rotations.
18. The method of examples 16 or 17 wherein the device stores data of the vibration applied to the pipe.
19. The method of any of examples 16-18 wherein the pipe includes a recess and the device is positioned in the recess.
20. The method of any of examples 16-19 wherein the device includes an accelerometer to measure vibration and a power source for powering the accelerometer.
21. The method of example 20 wherein the power source is a piezo chip.
22. The method of any of examples 16-21 wherein the pipe has a first end with a first cross-sectional area and a second end having a second cross-sectional area, the second cross-sectional area being smaller than the first cross-sectional area.
23. The method of example 22 wherein the device is positioned on the second cross-sectional area.
24. The method of example 23 that includes a recess wherein the device is in the recess.
25. The method of example 24 wherein the recess is in the second end.
26. The method of any of examples 16-25 wherein the device records the number of rotations of the pipe.
27. The method of any of examples 16-26 wherein the device records the vibration due to the material through the pipe is drilled.
28. The method of any of examples 16-27 wherein the device has a memory for recording the measured vibration.
29. The method of any of examples 16-28 wherein the device has a predetermined vibration quantity equal to the operational life of the pipe.
30. The method of example 29 wherein the measured vibration can be compared to the operational life to calculate the remaining life of the pipe.
31. The method of any of examples 16-30 wherein information from the device can be wirelessly extracted via a radio frequency signal.

What is claimed is:

1. An air monitoring array system comprising:
a plurality of air quality sensor devices arranged within a selected area, wherein the plurality of air quality sensor devices is configured to measure air pollutant levels in the selected area;
wherein each of the plurality of air quality sensor devices comprise:
at least one sensor operatively coupled to a controller, wherein the controller is configured to receive a measured input from the at least one sensor; and
a wireless communication device coupled to the controller, wherein the wireless communication device is configured to communicate with a central server.

2. The air monitoring array system of claim 1, wherein the central server is configured to determine if one or more portions of the selected area have air pollutant levels exceeding a predetermined threshold.

3. The air monitoring array system of claim 2, wherein the predetermined threshold is set by a government agency.

4. The air monitoring array system of claim 1, wherein the at least one sensor is at least one of an e-nose sensor circuit or a hydrocarbon sensor.

5. The air monitoring array system of claim 1, wherein the at least one sensor is configured to monitor benzene levels.

6. The air monitoring array system of claim 1, wherein each of the plurality of air quality sensor devices is powered by at least one of solar power or a battery.

7. The air monitoring array system of claim 1, further comprising a plurality of coordinators, wherein each of the plurality of coordinators is in communication with one or more sensor devices of the plurality of air quality sensor devices.

8. The air monitoring array system of claim 1, wherein each of the plurality of air quality sensor devices further comprises a temperature sensor for determining the ambient temperature at the at least one sensor.

9. The air monitoring array system of claim 1, wherein each of the plurality of air quality sensor devices further comprises an ultraviolet sensor for measuring ultraviolet levels at the at least one sensor.

10. The air monitoring array system of claim 1, wherein each of the plurality of air quality sensor devices further comprises an anemometer for measuring wind speed at the at least one sensor.

11. A method of air quality monitoring comprising:
measuring, by a plurality of air quality sensor devices arranged within a selected area, air pollutant levels in the selected area;
wherein each of the plurality of air quality sensor devices comprise:
at least one sensor operatively coupled to a controller, wherein the controller is configured to receive a measured input from the at least one sensor; and
a wireless communication device coupled to the controller, wherein the wireless communication device is configured to communicate with a central server.

12. The method of claim 11, further comprising determining, by the central server, if one or more portions of the selected area have air pollutant levels exceeding a predetermined threshold.

13. The method of claim 12, wherein the predetermined threshold is set by a government agency.

14. The method of claim 11, wherein the at least one sensor is at least one of an e-nose sensor circuit or a hydrocarbon sensor.

15. The method of claim 11, wherein the at least one sensor is a hydrocarbon sensor.

16. The method of claim 11, wherein the at least one sensor is configured to monitor benzene levels.

17. The method of claim 11, wherein each of the plurality of air quality sensor devices is powered by at least one of solar power or a battery.

18. The method of claim 11, further comprising communicating, by each of a plurality of coordinators, with one or more sensor devices of the plurality of air quality sensor devices.

19. The method of claim 11, further comprising determining, by a temperature sensor of each of the plurality of air quality sensor devices, the ambient temperature at the at least one sensor.

20. The method of claim 11, further comprising measuring, by an ultraviolet sensor of each of the plurality of air quality sensor devices, ultraviolet levels at the at least one sensor and/or measuring, by an anemometer of each of the plurality of air quality sensor devices, wind speed at the at least one sensor.
